(19) United States
(12) Patent Application Publication
(10) Pub. No.: US 2004/0086494 A1
(43) Pub. Date: May 6, 2004

(54) Immune privileged cells for delivery of proteins and peptides

(76) Inventor: Constance Mary John, San Francisco, CA (US)

Correspondence Address: Kenneth I. Kohn, KOHN & ASSOCIATES, Suite 410, 30500 Northwestern Hwy, Farmington Hills, MI, 48334, US

(21) Appl. No.: 09/941,398

(22) Filed: Aug. 28, 2001

Related U.S. Application Data
(63) Continuation-in-part of application No. 09/131,501, filed on Aug. 9, 1998, now abandoned.
Application No. 09/131,501 is a continuation-in-part of application No. 08/726,531, filed on Oct. 7, 1996, now abandoned.

(51) Int. Cl. A61K 48/00; C12N 5/08

(52) U.S. Cl. 424/93.21; 435/366

(57) ABSTRACT

Methods for sustained delivery of biologically active proteins or peptides to mammals are disclosed. Specific types of immune-privileged allogeneic or xenogenic donor cells that are naturally immune privileged are genetically modified in vitro to express or secrete the proteins or peptides. The genetically modified donor cells are subsequently implanted into host mammals and utilized for sustained delivery of biologically active proteins or peptides in vivo. The donor cells so utilized are those that inherently possess immune privilege due at least partly to the expression of Fas ligand. Methods for cell isolation, purification, tissue culture expansion, cryopreservation, gene transfer, transgene and Fas ligand expression, cell implantation, and measurement of immune responses of host animals are described.

### CROSS-REFERENCE TO RELATED APPLICATIONS

This is a continuation-in-part application under CFR 153(b) of U.S. Ser. No. 09/131,501 filed on Aug. 9, 1998 that was continuation-in part of U.S. Ser. No. 08/726,531 filed on Oct. 7, 1996.

### FIELD OF THE INVENTION

The present invention provides a method and composition for administration of a biologically active moiety by the use of mammalian cells that are naturally immune privileged, and that have been isolated and genetically modified so as to express the biologically active moiety in pharmacologically effective amounts. The biologically active moiety is not naturally expressed by the cells or is not expressed in pharmacologically effective amounts. More specifically, the invention employs in vitro genetic engineering of allogeneic and xenogeneic donor cells that are naturally immune privileged and then administering of the genetically modified cells to host mammals for sustained delivery of the biologically active moiety in vivo.

### DESCRIPTION OF THE RELATED ART

Immune privilege: Naturally occurring sites or tissues, such as the eye, testis, and the brain, that are immune-privileged were first described as such more than a century ago. Immune-privileged sites are regions of the body where grafts of foreign tissue survive for extended periods relative to sites that are not privileged. Grafts of immune-privileged tissues are more resistant to immune rejection than tissues that are not privileged. Expression of various molecules and multiple features has been found to contribute to maintenance of immune privilege (Streilein, 1995). Among those sites and tissues identified as immune-privileged are the anterior chamber of the eye, the cornea, retina, brain and peripheral nervous system, hair follicles, cartilage, liver, adrenal cortex, pregnant uterus, placenta, ovary, testis, prostate (Streilein, 1995). This invention provides a method and a composition for providing a biologically active moiety in vivo by administering cells from the tissues and sites that are naturally immune privileged, and that have been genetically modified to express the biologically active moiety in pharmacologically effective amounts in vivo.

Maintenance of immune privilege in these tissues has been thought to be variously associated with expression or secretion of a number of molecules, including immunosuppressive cytokines, corticosteroids, and Fas ligand, and the reduced or absent expression of class I and class II major histocompatibility complex molecules.

Different types of immune-privileged cells are biologically unique. Immune privilege is a complex property that is possessed by various naturally occurring cells and tissues that express multiple molecules that mediate the phenomenon. All of the naturally occurring immune-privileged cells express a multitude of molecules that are immunosuppressive as shown in Table 1. All immune-privileged cells apparently express Fas ligand. Nonetheless, there is significant variability in the expression of purported molecular mediators from one cell type to another (see Table 1). Thus, the different types of cells are biologically unique in the way that they create their immune-privileged status. This implies that the ability of the different types of cells to survive allogeneic implantation will vary. In addition, these cells are derived from various tissues of the body and, therefore, vary in their endogenous expression of other molecules and in their normal functional roles. The genetic modification of different cell types also varies and this is very significant for delivery of recombinant proteins in vivo.

Multiple molecular mediators produce immune privileged status. One likely molecular mediator of immune privilege is Fas ligand. Fas ligand, the naturally occurring ligand of Fas, was purified, cloned and identified as a protein of approximately 40,000 Mᵣ homologous to members of the tumor necrosis factor (TNF) family (Suda et al., 1993). Expression of

TABLE 1
Expression of Mediators of Immune Privilege in Mammalian Cells

| Molecular | | Retinal pigment | |
|---|---|---|---|
| mediator | Cytotrophoblasts | epithelial cells | Sertoli cells |
| Fas ligand | +(Runic et al., 1998; | +(Griffith et al., | +(Korbutt et al., |
| | Xerri et al., 1997) | 1995) | 1997; Xerri et al., |
| | | | 1997) |
| HLA-G | +(Houlihan et al., | −(Robert et al., | nd |
| | 1995; Kovats et al., | 1999) | |
| | 1990; McMaster et | | |
| | al., 1998) | | |
| Indoleamine | +(Takikawa et al., | +(Malina and | +(Ozaki et al., |
| 2,3- | 1988; Yamazaki et | Martin, 1993; | 1987; Ozaki et |
| dioxygenase | al., 1985) | Malina and | al., 1986) |
| | | Martin, 1996; | |
| | | Nagineni et al., | |
| | | 1996) | |
| Galectin-1 | +(Hirabayashi and | +(Allen et al., | +(Catt et al., |
| | Kasai, 1984; Poirier | 1990; Oda and | 1987; Wollina et |
| | et al., 1992) | Kasai, 1983) | al., 1999) |
| Galectin-3 | +(Lee et al., 1998; | +(Gupta et al., | −(Wollina et al., |
| | Phillips et al., 1996) | 1997) | 1999) |
| Interleukin- | +(Bennett et al., | nd | nd |
| 10 | 1997; Hennessy et | | |
| | al., 1999) | | |
| TGF-β | +(Lysiak et al., | +(Tanihara et al., | +(Caussanel et |
| | 1995; Sharma, | 1993) | al., 1997) |
| | 1998) | | |

CTB cytotrophoblasts; AEC amnionic epithelial cells; RPE retinal pigment epithelial cells;
nd = not done

recombinant Fas ligand on the surface of COS cells (a fibroblast-like kidney derived cell line) induced apoptosis in Fas-expressing cells within a few hours (Suda et al., 1993). Fas ligand does not have a signal sequence, but has a domain of hydrophobic amino acids in the middle and no signal sequence at the NH₂-terminus indicating that it is a type II transmembrane protein with the COOH terminal region outside the cell. Human and mouse Fas ligand have 76.9% homology and are functionally interchangeable (Takahashi et al., 1994). Human Fas ligand has been termed Apo-1 ligand while both Fas ligand and Apo-1 ligand are also referred to as CD95 ligand. The human Fas ligand contains 281 amino acids, and consists of a 79 amino acid cytoplasmic domain, a 23 amino acid transmembrane domain and a 179 amino acid extracellular domain.

Expression of Fas ligand in immune-privileged tissues or sites has been shown in tissues such as the testes, eye, spleen and thymus (Griffith et al., 1995). Cells that naturally express Fas ligand appear to possess immune privilege or produce specific immunological unresponsiveness. This was first demonstrated by Bellgrau et al. (Bellgrau et al., 1995; Selawry and Cameron, 1993). Sertoli cells from the testis of gld mice, a mutant strain of mice expressing a non-functional Fas ligand due to a point mutation, were rejected upon transplantation. Sertoli cells from the testis of normal and lpr mice, a mutant strain of mice that lack functional Fas but express normal Fas ligand, when transplanted under the kidney capsule of allogeneic animals were not rejected. Expression of Fas ligand by the testicular Sertoli cells was demonstrated by reverse transcription-polymerase chain reaction (RT-PCR).

Induction of apoptosis via the Fas-Fas ligand interaction also is a potent mechanism of immune privilege in the eye (Griffith et al., 1995). The results of Griffith and co-workers indicate that expression of Fas ligand triggers apoptosis in antigen-activated T cells that express Fas, and that constitutive expression of Fas ligand may be essential to maintenance of immune-privileged sites and tissues.

The eye is a privileged site that cannot tolerate destructive inflammatory responses or vision is destroyed. Activated neutrophils and lymphocytes entering the anterior chamber of the eye in response to a viral infection underwent apoptosis mediated by the Fas-Fas ligand interaction and did not cause tissue damage (Griffith et al., 1995). In contrast, viral infection in gld mice, a mutant strain of mice lacking functional Fas ligand due to a point mutation (Takahasi et al., 1994) resulted in inflammation and invasion of the eye by inflammatory cells that did not undergo apoptosis. Thus, immune privilege in the eye is maintained not through a passive process, but is an active process that induces cell death in potentially dangerous infiltrating cells by the Fas-Fas ligand interaction.

Griffith et aL using Northern (messenger RNA) blot and reverse transcriptasepolymerase chain reaction (RT-PCR) analysis of total RNA determined the expression of Fas ligand by the testis, thymus, eye and the spleen (Griffith et al., 1995). The location of Fas ligand in these tissues was also established immunohistochemically. Addition of competing Fas ligand peptide inhibited the binding of the antibody and verified the specificity of the antibody reaction within the tissue.

Another likely mediator of immune-privileged status is galectin-3. Galectin-3 is found on cytotrophoblasts and retinal pigment epithelial cells, but not on Sertoli cells. Galectin-3 is not a member of the Bcl-2 family, but at residues 180-183 it contains the four amino acid motif (NWGR) conserved in the BH1 domain of the bcl-2 family, and it has 48% sequence similarity with Bcl-2 (Yang et al., 1996). Consistent with this, galectin-3 has been found to be a novel antiapoptotic protein (Akahani et al., 1997; Yang et al., 1996), improving cellular adhesion and preventing apoptosis induced by loss of cell anchorage (anoikis) (Kim et al., 1999; Matarrese et al., 2000a), and influencing mitochondrial homeostatis (Matarrese et al., 2000b). Substitution of the Gly182 residue with Ala in the NWGR motif abrogates its antiapoptotic activity (Akahani et al., 1997; Kim et al., 1999). In T cells galectin-3 interacts with Bcl-2 in a lactose inhibitable manner and confers resistance to apoptosis induced by anti-Fas antibody and staurosporine (Yang et al., 1996). In the BT549 breast carcinoma cell line expression of galectin-3 inhibits cisplatin-induced apoptosis (Akahani et al., 1997).

Despite genetic differences mothers do not reject their partially allogeneic embryos. Since the advent of modern methods for in vitro fertilization and embryo implantation it has become clear that mothers are no more likely to reject fully allogeneic embryos. Cytotrophoblasts are of fetal origin but they normally exist within semi-allogeneic maternal tissue at the maternal-fetal interface. Thus, among those cells reported to possess immune privilege, cytotrophoblasts should possess the most developed ability to rebuff immune attack.

Expression of the products of the highly polymorphic class I HLA-A, -B and -C genes that stimulate graft rejection is blocked in the placental trophoblast cells that instead express HLA-G, a nonpolymorphic gene (Hammer et al., 1997; Hunt and Orr, 1992). This is a mechanism of inducing maternal tolerance. Expression of HLA-G has been cited as a possible mechanism for immune privilege of placental cells (Ellis et al., 1986; Kovats et al., 1990; McMaster et al., 1995), and recent in vitro studies support this theory (Carosella, 2000; Carosella et al., 1999; Lefebvre et al., 1999; Moreau et al., 1998; Rouas-Freiss et al., 1999; Rouas-Freiss et al., 2000; Sasaki et al., 1999a; Sasaki et al., 1999b). A number of laboratories have reported survival of implanted allogeneic trophoblasts or ectoplacental cones that contain trophoblastic cells without immunosuppression (Bevilacqua et al., 1991; Bowen and Hunt, 1999; Croy et al., 1984).

Fas ligand seems to play a role in the immune privilege of cytotrophoblasts, since cytotrophoblast-induced cell death of lymphoid cells in culture was partially inhibited by anti-FasL antibodies (Coumans et al., 1999). However, apparently normal pregnancies ensued in gld mutant mice that fail to produce functional Fas ligand and that were carrying trophoblast transgenic pups that abnormally expressed MHC class I in giant trophoblast cells (Rogers et al., 1998). Cytotrophoblast production of the cytokine IL-10 was found to be an important factor in maternal tolerance, and was immunoinhibitory in in vitro tests (Roth et al., 1996).

The ability of retinal pigment epithelium and corneal endothelium from mice to survive allogeneic implantation in a non-immune-privileged site was recently demonstrated (Hori et al., 2000; Wenkel and Streilein, 2000). RPE grafts from neonatal C57BL/6 or C57BL/6 gid mutant mice (deficient in functional CD95 ligand expression) were transplanted into the anterior chamber, the subretinal space, the subconjunctival space, and underneath the kidney capsule of BALB/c mice. The grafts from the normal C57BL/6 donors showed significantly enhanced survival at all sites compared with conjunctival grafts but the allogeneic gld grafts were rapidly rejected after transplantation beneath the kidney capsule (Wenkel and Streilein, 2000). When deprived of their epithelia, syngeneic corneas and allogeneic C57BL/6 corneas survived almost indefinitely beneath the kidney capsule (Hori et al., 2000).

Cytotrophoblasts, retinal pigment epithelial cells, and Sertoli cells all express indoleamine 2,3-dioxygenase (IDO), a tryptophan catabolizing enzyme, that appears to be critical in maintenance of maternal tolerance (Munn et al., 1998). In pregnant mice treated with 1-methyl-tryptophan, an inhibitor of IDO, rapid T-cell mediated rejection of all allogeneic pregnancies occurred. Syngeneic pregnancies of mice treated with the same inhibitor were not affected (Munn et al., 1998). The expression of IDO is regulated in human cells by interferons (Malina and Martin, 1996); the most efficient of these is interferon-□IFN-□. Interferons have anti-cancer activity and can inhibit tumor cell growth in culture (Taylor and Feng, 1991). It has been shown in vitro that a primary mechanism of the cytotoxicity of IFN-□ is the induction of IDO. IDO uses two superoxide radicals to cleave the pyrrole ring of tryptophan, an essential amino acid, in the first, and rate limiting step of tryptophan catabolism, and is an antioxidant enzyme (Malina and Martin, 1996). It is now well established that tryptophan starvation resulting from IFN-□treatment is the mechanism of the antiproliferative activity of IFN-□ on many cell lines and intracellular parasites (Taylor and Feng, 1991). Tryptophan starvation can lead to apoptosis of cells. Within 48 h of treatment with IFN-□ ME180 human epidermoid carcinoma cells underwent apoptosis that could be prevented by adding tryptophan and induced by removing it in the absence of IFN-\tilde{□} Replication of the parasite Toxoplasma gondii was inhibited by treatment of infected RPE cells in culture with IFN-\tilde{□} The inhibition could be reversed by addition of tryptophan (Nagineni et al., 1996).

The number of different immunosuppressive molecules immune-privileged cells express suggests that modifying non-immune-privileged cells to express one single molecular mediator could be insufficient to achieve allogeneic survival in vivo without immunosuppressive drugs. Modification of cells with genes encoding individual molecules mediating immune privilege to artificially transfer the property to non-immune privileged cells does appear to be an approach with serious limitations.

Reports of the role of Fas ligand in maintenance of immune privilege stimulated research in the transgenic expression of FasL on the allogeneic cells to prevent rejection. Fas ligand induces apoptosis of cells, including activated lymphocytes, that express its receptor, Fas (CD95/APO-1), and prevents inflammatory reactions at immune privileged sites by triggering Fas-mediated apoptosis of infiltrating proinflammatory cells. The initially promising reports (Bellgrau et al., 1995; Griffith et al., 1995; Lau et al., 1996) were followed by a number of reports of failures to achieve tolerance using transgenic Fas ligand (Allison et al., 1997; Chervonsky et al., 1997; Kang et al., 1997). An increasing number of studies have shown that Fas ligand can induce potent inflammatory responses that appear to limit its ability to inhibiting graft rejection (Kang et al., 1997; O'Connell, 2000; Ottonello et al., 1999; Turvey et al., 2000).

Chervonsky and coworkers demonstrated that expression of transgenic Fas ligand on allogeneic β-islet cells caused rejection due to Fas-mediated destruction of the islet cells themselves (Chervonsky et al., 1997). Normally islet cells do not express Fas, but contact with cells expressing Fas ligand can lead to upregulation of Fas on islet cells, and the capacity for Fas upregulation increases with age. Chervonsky et aL concluded that Fas-mediated apoptosis of islet cells may play a major role in development of Type 1 (autoimmune) diabetes.

In a review of this area, Green and Ware (Green and Ware, 1997) discuss several other possible explanations for the variation in the results with transgenic Fas ligand; such as the age of donor animals, or factors in the host at the specific transplantation site such as interferon γ or IL-8. The amount of soluble Fas ligand secreted may vary among Fas ligand-expressing cells. The secreted form of Fas ligand is a monomer, unlike the membrane bound protein, that is trimerized when functional. Perhaps varying amounts of recombinant Fas ligand on the cell membranes is significant in terms of tipping a balance between local inflammation and immunoprotection. Recently human recombinant soluble Fas ligand has been found to be endowed with potent chemotactic properties toward human neutrophilic polymorphonuclear leukocytes (neutrophils) (Ottonello et al., 1999). Also, naturally immune-privileged cells may express other molecules that are critical for induction of immune privilege.

A colon carcinoma cell line, CT26, was stably transfected with Fas ligand (CT26-CD95L). When injected in syngeneic Balb/c mice subcutaneously, the CT26-CD95L cells were rejected by neutrophils activated by Fas ligand. However, CT26-CD95L survived in the intraocular space because of the presence of transforming growth factor-beta (TGF-beta) that inhibited neutrophil activation. Importantly, providing TGF-beta to the subcutaneous sites prevented rejection of the tumor at those sites. Thus, Fas ligand together with TGF-beta was able to promote immunologic tolerance of the tumor cells but expression of Fas ligand alone was not able to do so suggesting that together these cytokines generate a microenvironment that promotes immune tolerance that could prevent allograft rejection (Chen et al., 1998).

Similar conclusions can be drawn from recent work with Sertoli cells from non-obese diabetic (NOD) mice, a model for autoimmune Type 1 diabetes. The NOD Sertoli cells were implanted under the right renal capsule of diabetic NOD mice, whereas NOD islets alone were implanted under the left renal capsule (Suarez-Pinzon et al., 2000). After 60 days 9 of 14 mice that received islet and Sertoli cells grafts were normoglycemic compared to 0 of the 6 mice that received islet grafts alone. Immunohistochemistry revealed that TGF-beta expression by the grafted Sertoli cells was high, but the expression of Fas ligand decreased after transplantation. Administration of anti-TGF-beta antibody completely abrogated the protective effect of Sertoli cells on islet graft survival, whereas anti-Fas ligand antibody did not. (Suarez-Pinzon et al., 2000). The expression of TGF-beta was critical in the ability of the Sertoli cells to prevent death of the islets due to the autoimmune condition of the NOD mice. Together these data demonstrate the complexity of the phenomenon termed immune privilege, and the fact that it could be difficult to recreate it by recombinant expression of a single molecule mediator. In addition, the data suggest that assays that measure the amount of TGF-beta secreted by immune-privileged cells in vitro and in vivo could be useful to determine the population of cells that would be most effective in warding off rejection by the host immune system in allogeneic implants. We plan to compare the allogeneic survival of various types of immune privileged cells with the levels of TGF-beta they secrete. The complexity and variety of the means that nature has used to create the immune privileged status of particular cells are indications of the difficulty of achieving this status.

The use of the genetically modified cells that are naturally immune privileged is a practical and novel method for ex vivo gene therapy and protein drug delivery. In this method we do not attempt to dissect and then reassemble what nature has provided, but rather to make use of it in a novel manner. Our in vitro study of the immunosuppressive effects of naturally occurring murine immune privileged cells has revealed characteristics that make trophoblasts more suitable for delivery in some parts of the body Sertoli cells. We postulate that this type of assay and other in vitro assays such as determination of TGF-beta secretion will reveal measurable differences between the cell types that will aid in identification and characterization of cells that will be significantly more useful as in vivo drug delivery vehicles than other types of immune-privileged cells.

In in vivo studies we are comparing the survival of different types of allogeneic immune-privileged cells and the immune response of the host animal to the cells. We postulate based on our in vitro studies that some of these types, such as trophoblast cells, will be better able to survive and cause less immune and inflammatory reactions from the host.

Transplant Rejection: Transplantation of healthy organs or cells into a mammal suffering from a disease that affects the organ or cells may be necessary to save the mammal's life. A major problem in transplantation or implantation of any foreign tissue or cell is immune-mediated graft rejection in which the recipient's T-lymphocytes recognize donor histocompatibility antigens as foreign. Thus, use of non-autologous human (allografts) and or mammalian (xenografts) cells requires preventing immune rejection by the host. The donor and recipient are matched as closely as possible to prevent rejection in transplantation of humans. Survival of even well-matched grafts often necessitates high dose chronic treatment with nonspecific immunosuppressive drugs that can result in opportunistic infections and in the majority of transplant patients, long term complications (Gjertson, 1991; Manninen et al., 1991). Rejection is still a leading cause of graft failure, despite progress in immunosuppressive therapy.

Both TGF-beta and Fas ligand along with CD8 accessory molecule, and the major histocompatibility class I gene products, have been directly implicated in the mechanism of the “veto effect”, that is deletion of graft reactive T cells by administration of low doses of donor bone marrow cells. This method to induce transplantation tolerance for allografts without chronic immunosuppression is close to clinical use. It involves transient peritransplant depletion of host T cells followed by intravenous administration of a low dose of donor bone marrow cells (George and Thomas, 1999).

Delivery of Therapeutic Proteins: Over the last 15 years the application of recombinant DNA technology in the pharmaceutical field gave rise to the entire biotechnology industry. A distinct advantage of biotechnology-derived proteins over those isolated from natural sources is enhanced purity. Obstacles to the use of proteins as therapeutic agents include the propensity to aggregate, adhere to surfaces, become denatured and rapidly metabolized. These have been at least partially overcome, and increasing numbers of protein products are on the market. New protein-based pharmaceuticals that have and are arising from biotechnology processes include a wide spectrum of pharmacologically active substances, such as hormones, hormone-like regulatory compounds, enzyme inhibitors, vaccines, and antibodies.

Few protein biopharmaceuticals can be successfully administered orally because of their instability in the acidic environment of the stomach and the barrier to absorption presented by the gastrointestinal tract (Hudson and Black, 1993). Rapid metabolism by a multitude of enzymes and nonlinear pharmacokinetics are other challenges in the delivery of protein and peptide drugs (Wearley, 1991).

Optimally a drug or biologic substance is delivered to the site of pharmacologic action, is able to penetrate the biologic barriers, and access the site of action, either intra- or extracellular, in therapeutically effective doses (Bruck, 1991). The field of controlled drug delivery may be divided in four categories as follows: (1) non-specific or non-targeted drug release systems such as polymeric diffusion systems and infusion pumps; (2) pharmaceutical formulations including various coatings to sustain action of drugs; (3) prodrugs that can undergo transformation in the body before eliciting their pharmacologic effects; and (4) targeted delivery of drugs and biologicals via carriers, such as liposomes, biodegradable polymers, antibodies, and genetically engineered cells.

Most protein products are delivered by invasive routes such as intravenous (i.v.), intramuscular (i.m.), or subcutaneous (s.c.). This is an obvious disadvantagetheir delivery can be associated with some risk and cause minor discomfort. Until new dosage forms are developed, the availability of proteins in the ambulatory setting is limited. However, some methods to minimizing the remaining obstacles of non-invasive protein/peptide drug delivery have been found.

The methods for enhancing protein delivery include increasing the absorption, minimizing metabolism and prolonging the half-life of the protein (Wearley, 1991). Administration of either enzyme inhibitors or protective polymers and permeation enhancers can improve the bioavailability of proteins and peptides delivered by noninvasive means. However, the bioavailability may still remain fairly low.

Nasal administration of nonpeptides and peptides of ten residues or less has been quite successful. Examples include oxytocin, vasopressin and desmopressin acetate and luteinizing hormone-release hormone and its superanalogs buserelin, leuprolide and nafarelin. However, when the number of amino acids is increased to 20 or greater, as in insulin, glucagon, or growth hormone releasing hormone, low bioavailability is the result, except when delivered with a penetration enhancer. Other drug delivery routes for proteins studied include transdermal, buccal, rectal, respiratory and ocular (Wearley, 1991).

Most regulatory proteins, such as insulin and growth hormone, must reach distant organs or tissue without being extensively metabolized. Unique ways to achieve this goal include encapsulating the protein-based compounds in lipid complexes such as liposomes, protecting proteins in a sheath composed of poorly soluble biopolymers such as polyethylene glycol, fusing the protein with antibodies that can be directed to distinct tissue, and using the body's own cells as carriers. The use of gene-carrying cells as “factories” that produce the desired protein in a targeted tissue is very promising (Hudson and Black, 1993). Cells themselves have been used to deliver protein-based toxins to malignant cancer cells. For example, in the first clinical trial of gene therapy, tumor-invading T-lymphocytes were engineered to secrete tumor necrosis factor, which is cytotoxic (Ledley, 1989; Rosenberg et al., 1990).

Gene Therapy: Human gene therapy began in the 1950s and 1960s when successful renal transplantation lead to the concept that injecting healthy cells into patients with genetic diseases might be therapeutic (Brady, 1966). Initial clinical studies were undertaken in the 1970s with transplantation to treat Gaucher disease (Groth et al., 1972) and Hunter syndrome (Dean et al., 1975) and the term ‘gene therapy’ was coined (Friedmann and Toblin, 1972).

Gene therapy is an approach to human disease based on the transfer of genetic material (DNA) into an individual. This can be achieved by direct administration of DNA or DNA-containing viruses to blood or tissues (in vivo), or indirectly through the introduction of cells engineered to contain foreign DNA (ex vivo) (Orkin and Motulsky, 1995). Only the somatic cells and not the germ cells (eggs and sperm) are the targets of gene therapy efforts. Until recently, most of the work in human gene therapy centered on rare genetic diseases. However, gene therapy may be appropriate in a variety of clinical settings, such as:

1) single-gene inherited disorders such as delivery of normal factor VIII genes to patients with hemophilia;

2) common, multifactorial disorders such as coronary heart disease;

3) cancer by correction of mutations in tumor suppressor genes, e.g. p53, or approaches such as delivery of genes encoding enzymes involved in conversion of prodrugs to active form, and

4) infectious diseases such as HIV.

Diseases that are currently treated by the administration of proteins may be amenable to treatment by gene therapy, and in these cases gene therapy can be thought of as an in vivo protein production and delivery system.

The first human patients received gene therapy at the NIH in 1991. As of June 1995 there have been 106 clinical protocols involving gene transfer in humans approved by the NIH Recombinant DNA Advisory Committee (RAC), and a total of 597 human subjects have undergone gene transfer experiments. Estimated expenditures on gene therapy research by the NIH and biopharmaceutical industry are over $400 million a year (Hanania et al., 1995; Orkin and Motulsky, 1995). However, despite promising results in animals, clinical efficacy has not been definitively shown in a gene therapy protocol in humans.

Major problems in this field include the following:

1) inability to achieve efficient gene transfer;

2) lack of persistence in gene maintenance and expression;

3) inability to achieve expression in appropriate tissues and cells;

4) immunorejection after introduction of genetically modified allogeneic or xenogeneic cells (Tai and Sun, 1993);

5) inadequate understanding of the interactions of the vectors with the host, and

6) lack of understanding of the results of gene therapy protocols, which are hindered by a low frequency of gene transfer, reliance on qualitative assessments of transfer and expression, lack of suitable controls and rigorously defined endpoints (Orkin and Motulsky, 1995).

Vector systems that currently have been used or are under consideration for use in gene therapy include retrovirus, adenovirus, adeno-associated virus, herpes virus, pox virus, naked DNA and facilitated DNA (Orkin and Motulsky, 1995). Methods being explored to deliver DNA include particle bombardment (also known as ballistic, microprojectile or gene gun method), electrically-induced DNA transfer, calcium phosphate-mediated DNA transfection, liposomal and receptor-mediated gene delivery (Bennett et al., 1994; Wolff, 1994).

Many protocols approved for somatic-cell gene therapy do not involve direct administration of the genetic vector, but rather are ex vivo strategies that require the isolation of somatic cells from a patient, the stable introduction of a gene of therapeutic interest into the cells, the isolation and clonal propagation of a single engineered cell, and finally, the reintroduction of the cells into the patient (Heartlein et al., 1994; Kessler et al., 1993). An ex vivo strategy ensures that the genes are delivered to the cells of the right tissues or organs.

Human clinical trials of gene therapy for specific diseases have been performed or have commenced in areas including cancer vaccines, genetic sensitization trials in which sensitization to ganciclovir is conferred by transfection with herpes virus thymidine kinase, gene replacement trials with the adenosine deaminase gene for severe combined immmunodeficiency disease, (SCID), for Gaucher's disease in which the glucocerebrosidase gene is dysfunctional, and in cystic fibrosis with cDNA to replace the cystic fibrosis transmembrane conductance regulator (CFTR) to replace the gene that is missing in these patients (Hanania et al., 1995). Another strategy being explored with gene therapy is that of chemoprotection for autologous bone marrow transplantation and chemotherapy sensitization with anti-oncogenes such as administration of a vector containing a functional wild-type p53 transcription unit.

Transkaryotic implantation is a term for isolation of somatic cells from a patient, introduction of a gene of therapeutic interest, isolation and clonal propagation of a single engineered cell, and finally, reintroduction of the cells into the patient. The use of nonimmortalized clonal strains of secondary and primary cells may offer significant advantages, besides lacking tumorigenicity, these cells are more likely to maintain differentiated functions than immortalized cells. Transkaryotic implantation circumvents the problem of immunorejection by the transfection of autologous cells for reintroduction into the original donor. Transkaryotic implantation will tend to be costly and labor intensive.

Another approach, termed microencapsulation, has been designed to circumvent the problem of immunorejection of genetically engineered allogeneic or xenogeneic cells from “universal” cell lines (Al-Hendy et al., 1995; Hughes et al., 1994; Squinto et al., 1994; Tai and Sun, 1993; Uludag and Sefton, 1992; Wang et al., 1991). A feature of this method is the prevention of immunorejection by physical isolation of the implanted cells from the host (recipient) immune system by enclosure within microcapsules. The membranes are designed to provide free passage for the recombinant protein products. Mice transplanted with encapsulated transformed fibroblasts secreting human growth hormone (hGH) had detectable levels of hGH over 115 days, the course of a recent study (Tai and Sun, 1993). Approximately 60% viability was observed among encapsulated myoblasts retrieved after secreting mouse growth hormone for six months in Snell dwarf mice, that had enhanced growth (Al-Hendy et al., 1995). Potential problems of this approach include the eventual breakdown of the capsule, the need for high-level product secretion in some cases, and difficulty in achieving long-term survival of encapsulated cells. The pores in the capsules allow for diffusion of recombinant protein products out, but also allow antigenic proteins from dying cells out, and allow the diffusion in of host proteins and molecules, such as IL-1 (17 kDa), TNF-□ (17-51 kDa), IL-6 (26 kDa), oxygen radical, and nitric oxide (Babensee et al., 1998; Hagihara et al., 1997; Rihova, 2000). Immune responses to encapsulated cells are well documented and are greater for xenogeneic cells (Babensee et al., 1998; Rihova, 2000).

Transgenic Animals. Many human therapeutic proteins are currently produced on a large scale with the aid of recombinant DNA technology in microbial bioreactors and a few in animal cell cultures. A disadvantage of the microbial production of therapeutic proteins is that while microbes such as bacteria and yeast do translate the genetic code into the correct amino acid sequence, they do not necessarily add the correct post-translational modifications such as glycosylation which takes place in the Golgi apparatus of eukaryotic cells or fold the protein to yield the ultimately biologically active product. While the actual production of proteins from microbial bioreactors may be inexpensive, purification and processing of the proteins tends to be costly. Animal cell culture can circumvent some of these problems, but it tends to be prohibitively expensive due to long generation times and requirement for rich media. Systems that have been used to produce recombinant proteins include bacteria, yeast, fungi, plants, baculovirus, mammalian cells and transgenic animals.

Another possible alternative is the manufacture of proteins in animals, which requires transferring foreign genes into the animals' embryos. If the foreign gene is introduced into the one-cell embryo (fertilized oocyte), and integrated, the transgene becomes a dominant Mendelian genetic characteristic that is inherited by the progeny of the founder animals. The ability to genetically manipulate mammals has opened an immense potential with almost unlimited applications in basic and applied research, and the production of human pharmaceuticals in transgenic animals has become more attractive. With targeted gene transfer, the expression of the transgene of interest can be directed, for example, to the mammary gland so that the protein is secreted into the milk (Janne et al., 1992).

Considerable progress has been made in targeting tissue-specific expression to the mammary gland and the blood of animals. For example, human proteins have been produced in the milk or blood of transgenic mice, rabbits, sheep, pigs and goats. These proteins include factor IX, alpha-1 antitrypsin, t-PA, antithrombin III, protein C, and human growth hormone (Hoyer et al., 1994). This approach has great potential productivity. If similar yields of Factor IX could be obtained in pigs as were produced of human protein C, a vitamin K-dependent plasma protein, then twenty pigs transgenic for Factor IX could easily produce the two kg of protein that is used each year in the US (Hoyer et al., 1994).

No recombinant proteins extracted from transgenic animals are yet on the market (Houdebine, 1994), however, there is relatively slow but real progress being made in improving the efficiency of this process. Predictive reports suggest that 10% of the recombinant proteins, corresponding to a $100 million annual market, will be prepared from the milk of transgenic animals by the end of the century.

Deficiency of prior art. The prior art is deficient in a simple, reliable method for cell-based “gene therapy” that would enable sustained, systemic delivery of proteins and peptides in vivo with little or no need for chronic immunosuppression to prevent rejection. This type of approach has been termed nonautologous somatic gene therapy (Al-Hendy et al., 1995). The present invention will lead to the development of a convenient method for the sustained, systemic delivery of proteins, glycoproteins, and peptides by genetically modified cells that are naturally immune privileged to fulfill a long-standing need (FIG. 1).

Research and development of gene therapy is a very active field and includes numerous clinical trials in human beings. Nonetheless, difficulties have been encountered and despite promising results in animals, clinical efficacy has not been definitively shown in a gene therapy protocol in humans. The invention described here would enable development of “universal” cell lines that could be thoroughly characterized for safety and quality assurance before implantation. In comparison, thorough characterization of transfected autologous cells for somatic cell gene therapy would be costly and time-consuming. This method obviates the need for patient specific genetic manipulation and is amenable to industrial scale quality control. Thus, accurate analysis of the efficiency of the gene transfer, and the persistence of gene maintenance and expression will be possible. The invention would help ensure the clinical success of cell-based gene therapy and, therefore, greater reflection of the promising results obtained in animal studies (Friedmann et al., 1994).

Prior art related to this invention includes a large number of patents for transfected cell lines, transgenic animals, and human gene therapy, including a broad-based patent issued by the U.S. Patent and Trademark Office PTO covering ex vivo gene therapy (Anderson et al., 1995).

Additional prior art to the current invention includes foreign patent document WO9528167 entitled “Methods of Treating Disease Using Sertoli Cells and Allografts or Xenografts,” invented by Helen P. Selawry published on Oct. 26, 1995 which describes a method to create an immune-privileged site in a recipient mammal using Sertoli cells. The method relies on cotransplantation of allogeneic or xenogeneic cells that produce a desirable biological factor together with immune-privileged Sertoli cells to prevent rejection. The use of the genetically-modified Sertoli cells or other immune-privileged cells that are naturally immune privileged to produce and deliver peptides and proteins was not envisioned or described in U.S. Pat. No. 5,579,534. One drawback to the method described in U.S. Pat. No. WO9528167 is that more than one cell type must be administered for therapy.

Similarly, Gage et al. in U.S. Pat. No. 5,082,670 entitled “Method of Grafting Genetically Modified Cells to Treat Defects, Disease or Damage Of the Central Nervous System” published on Jan. 21, 1992 in claim 24 describe the coadministration of cells (along with the genetically modified donor cells of claim 1) as a therapeutic agent for treating disease or damage to the central nervous system, said therapeutic agent consisting of cellular matter, including homogenate of placenta. Also, in claim 26, Gage et al. describe implantation of cellular material into the central nervous system (along with the genetically modified donor cells of claim 1) to facilitate reconnection or ameliorative interaction of injured neurons, said cellular material including homogenate of placenta (Gage et al., 1997). A continuation application for U.S. Pat. No. 5,082,670 was published on Jul. 22, 1997 as U.S. Pat. No. 5,650,148. U.S. Pat. No. 5,082,670 or the continuation U.S. Pat. No. 5,650,148 does not anticipate or describe genetic modification of the co-implanted cellular material or matter described in claims 24 or 26, such as placental cells. Likewise, the use of unmodified immune-privileged cells that are naturally immune privileged and naturally express Fas ligand, such as placental cells, for therapy or implantation in vivo is not described in the present invention.

Another patent related to the current invention is U.S. Pat. No. 5,759,536 entitled “Use of Fas ligand to suppress T-lymphocyte-mediated immune responses,” invented by Donald Bellgrau and Richard C. Duke and published on Jan. 7, 1995. U.S. Pat. No. 5,759,536 describes the use of cells or tissues that have been genetically modified to express recombinant Fas ligand, or therapy with recombinant Fas ligand protein itself. The use of genetically-modified immune-privileged cells that naturally express Fas ligand to deliver peptidic biomolecules was not envisioned or described in U.S. Pat. No. 5,759,536.

U.S. Pat. No. 5,702,700 entitled “Sertoli cells as neurorecovery inducing cells for Parkinson's Disease” dated Dec. 30, 1997 by the inventors Paul R. Sanberg, Don F. Cameron, and Cesario V. Borlongan was not uncovered in the searches performed prior to the Oct. 7, 1996 filing of the original patent application Ser. No. 08/726,531 describing the present invention. U.S. Pat. No. 5,702,700 describes the therapeutic use of trophic factors that are naturally secreted by Sertoli cells by implantation into the central nervous system of mammals. Testis-derived Sertoli cells have been shown to have a trophic effect on dopamine neurons and alleviate hemiparkinsonism in rats (Sanberg et al., 1997). Recent work in this area demonstrated survival of rat Sertoli cells allografts and porcine Sertoli cell xenografts for at least two months in the rat brain without cyclosporin A immunosuppression (Saporta et al., 1997). The use of genetically-modified cells that are naturally immune privileged, such as Sertoli cells, to deliver desired peptidic biomolecules, either in vitro or in vivo, was not envisioned or described by U.S. Pat. No. 5,702,700. Likewise, the use of unmodified immune-privileged cells that naturally express Fas ligand, such as Sertoli cells, for therapy or implantation in vivo is not described in the present invention. The survival of allogeneic and xenogeneic Sertoli cells in rat brain without systemic immunosuppression provides dramatic evidence of the ability of naturally immune-privileged cells to prevent immunorejection in a mammalian central nervous system (Saporta et al., 1997).

### SUMMARY OF THE INVENTION

The present invention provides a method for delivery of a biologically active moiety by administering immune-privilege cells that have been genetically modified to express the biologically active moiety. The biologically active moiety is provided in vivo in pharmacologically effective amounts, and is either not naturally expressed by the immune-privileged cells, or is naturally expressed in amounts that are less than required for pharmacologically effectiveness. The biologically active moiety could be a protein, peptide, gene, or the product of a protein such as a neurotransmitter, and could be expressed as a pro-drug that is activated in the body. The administration of the immune-privileged cells can be performed by a variety of methods including subcutaneous, intravenous, intraperitoneal, and intramuscular infusion or injection. Additionally, the immune-privileged cells could be implanted in specific sites of the body by a number of surgical procedures. The cells could be adherent to an inert polymeric material that would keep them together at a specific location in the body. The cells could be implanted in a polymeric material that is a liquid that gels upon implantation in the body so that the cells are retained at the site of implantation. The expression of the biologically active moiety could be either intracellular, on the extracellular membrane, or secreted by the cells depending on where the biologically active moiety would be therapeutic. The immune-privileged cells could be freshly isolated cells, or cells that have been cultured, or that have been cultured and then frozen. The immune-privileged cells could be, or could be derived in culture from, progenitor stem cells of immune-privileged cells. Immune-privileged cells are those that naturally express Fas ligand and that have greater ability to survive allogeneic grafting or implanting with less immunosuppression than other non-immune privileged cells of the body. A listing of cells that express Fas ligand is given in Table 2. The genetic modification of the cells can be performed by the various methods that are known to one of skill in the art. Promoters could be incorporated into the genetic modification of the immune-privileged cells such that the biologically active moiety will be expressed when the promoter is turned-on by the administration, oral or otherwise, of a drug molecule such as tetracycline. The method used for genetic modification could be inserting a transgene with one or more viral vectors. In addition, the genetic modification could be performed by nonviral physical methods such as microinjection, electroporation, lipofection, and chemically-mediated transfection with calcium phosphate or liposomes, and other methods known to one of skill in the art. The pharmacologically effective amount is defined by therapeutic indices or responses appropriate to the disease state or condition that is being treated.

### BRIEF DESCRIPTION OF THE DRAWINGS

So that the matter in which the above-recited features, advantages and objects of the invention, as well as others which will become clear, can be generally attained and understood in more detail, more particular descriptions of the invention briefly summarized above may be had by reference to certain embodiments thereof which are illustrated in the appended drawings. These drawings form a part of the specification, and illustrate general embodiment of the invention but are, therefore, not to be considered limiting in their scope.

FIG. 1 illustrates the methodology employed to create transgenic immune-privileged cells naturally expressing Fas ligand (FasL) for the expression and secretion of proteins, glycoproteins, and peptides.

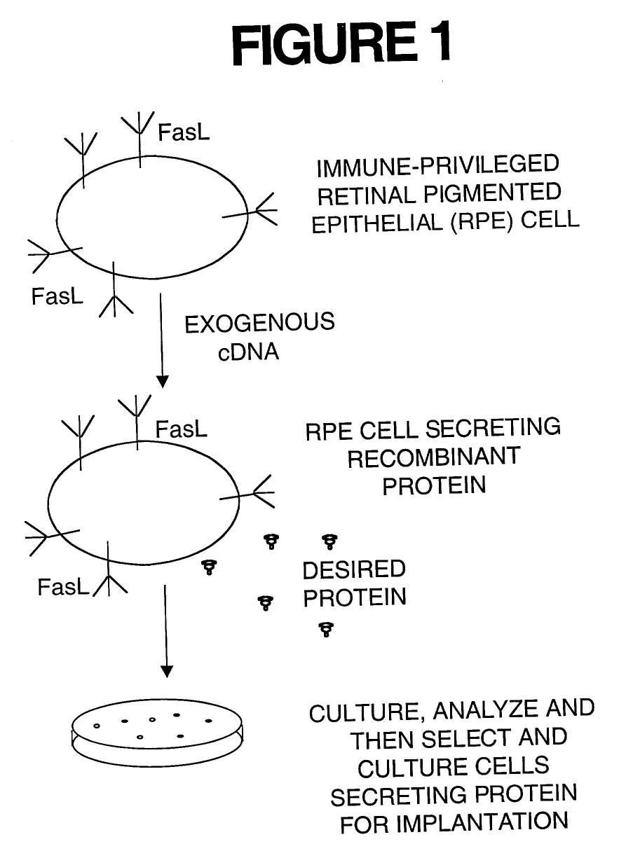

FIG. 2 represents the various elements of deoxyribonucleotide (DNA) sequences that can be used in a vector to obtain optimized expression of a transgene, particularly in transgenic animals. In some cases, incorporation of the gene in specific cell types can be obtained. Terms and abbreviations used are as follows: SCS—specialized chromatin structure sequences capable of insulating genes and transgenes; MAR—DNA linked to the nuclear matrix through matrix attached regions or scaffold attached regions (SAR) some of which are involved in the control of DNA replication and segregation, or gene expression; LCR—locus control region confers position-and copy number independent expression on genes under its control; Enhancer—for many genes, tissue specificity and high expression is regulated by enhancers; Promoter—regulates the transcription of the cDNA into messenger RNA; Leader—also named 5′ untranslated region or 5′ UTR, may favor more or less translation and needs to be at least 77 nucleotides for maximum efficiency; Introns—non-coding regions of DNA that seem to contain multiple signals of unknown nature which govern the status of a gene during development; 3′ UTR—3′ untranslated region that participates in some cases in the stabilization of messenger RNA; Terminator—transcription terminator, that may have quite variable potency depending on the construct in which it is inserted.

FIG. 3 presents an overview of the methodology for the production and evaluation of transgenic pigs, and was adapted from M. J. Martin and C. A. Pinkert, 1994 (Martin and Pinkert, 1994).

FIG. 4 is a map of the PLNCS retroviral vector (Clontech, Laboratories, Inc.) with multiple cloning site (MCS). PLNCX was derived from Moloney murine leukemia virus and is designed for retroviral gene delivery and expression. Upon transfection into a packaging cell line, pLNCX can transiently express, or integrate and stably express a transcript containing Ψ, the extended viral packaging signal, a selectable marker, and the gene of interest. The vector RNA is recognized by viral proteins and packaging into retroviral particles.

FIG. 5 is a schematic of a pNT3/LNCX retroviral vector for expression of recombinant neurotrophin-3 (NT-3).

FIG. 6 illustrates the packaging of retroviral particles with a packaging cell.

FIG. 7 illustrates viral infection and characterization of porcine retinal pigment epithelial (RPE) cells secreting NT-3 and grafting into a rat model of spinal cord injury.

FIG. 8 is a stick diagram depicting the rat androgen-binding protein (ABP) promoter construct engineered to drive expression of the human growth hormone transgene (hGH) in Sertoli cells. The ABPp/hGH plasmid contains the ABP promoter and the entire 2.1 kb hGH gene, including introns, 3′ untranslated sequence, and the polyadenylation signal.

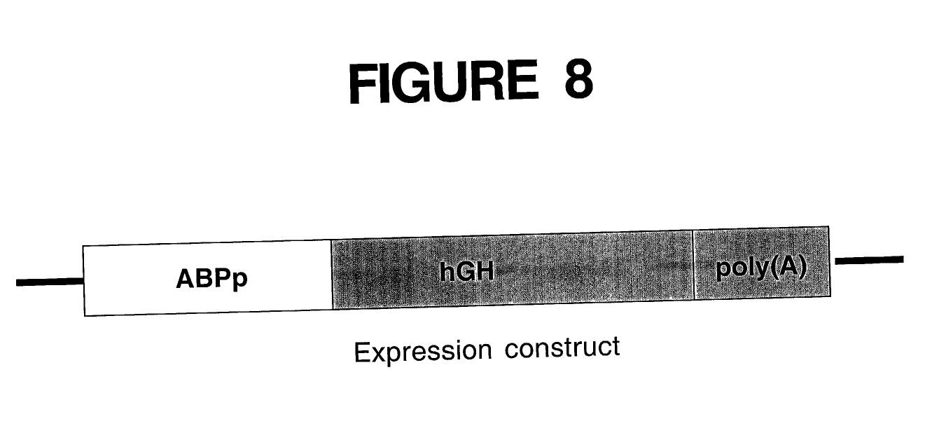

FIG. 9 (A) depicts the functional effect of spinal cord lesions in rats on the number of footfalls per grid task, (B) depicts the functional assay of normal and lesioned animals treated with NGF and NT3 and (C) the increased corticospinal tract sprouting obtained from grafting NT-3 secreting fibroblasts into lesioned animals. Significant decreases in the number of footfalls per grid task were also observed in the NT-3 grafted lesioned animals. The data and figure were adapted from Grill et al. 1997 (Grill et al., 1997).

FIG. 10 is a schematic of the tyrosine hydroylase/LNCX retroviral vector under the control of the retinal pigment epithelium (RPE)-65 promoter.

FIG. 11:Fluorescence microscopy of rat Sertoli cells stained red with different concentrations of antibody to the follicle-stimulating hormone receptor (FSHr). From 0 (A), 2 (B), 4 (C) to 8 □g/ml of antibody was used to stain the cells.

FIG. 12: Rat Sertoli cells infected with replication incompetent adenovirus. Sertoli cells were infected with Ad5eGFP as per the conditions described above. Three days after the infection, the cells were examined for green fluorescent protein expression using a fluorescence microscope. In the left panel cells were infected with 104 particles/cell and in the right panel they were infected with 10⁵ particles/cell.

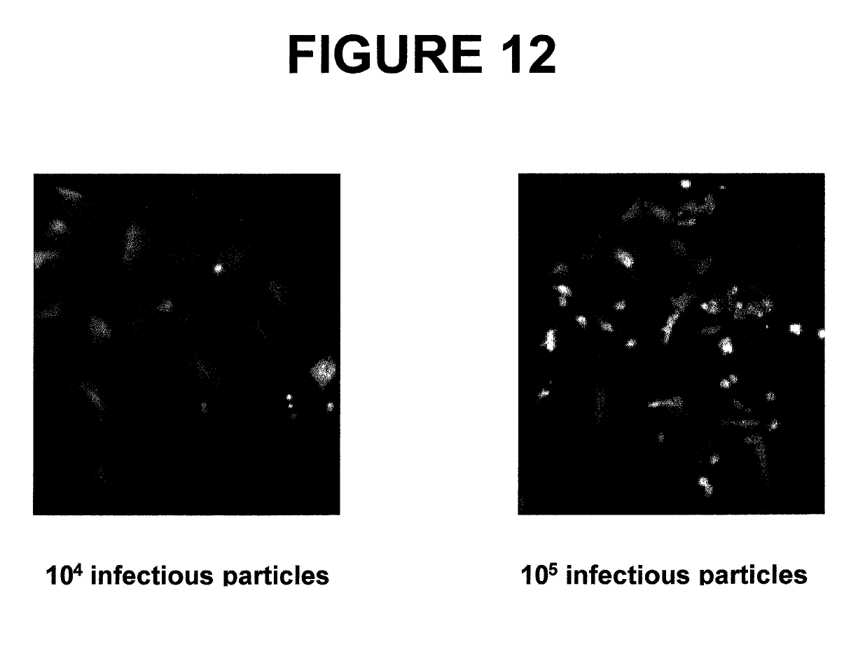

FIG. 13: Adenoviral expression vector. Human NT-3 was inserted, in the correct orientation, into the multiple cloning site of the adenoviral expression plasmid. The expression of the gene was under the CMV promoter and expression was terminated by polyA sequences. Restriction enzyme sites used for directional cloning are indicated.

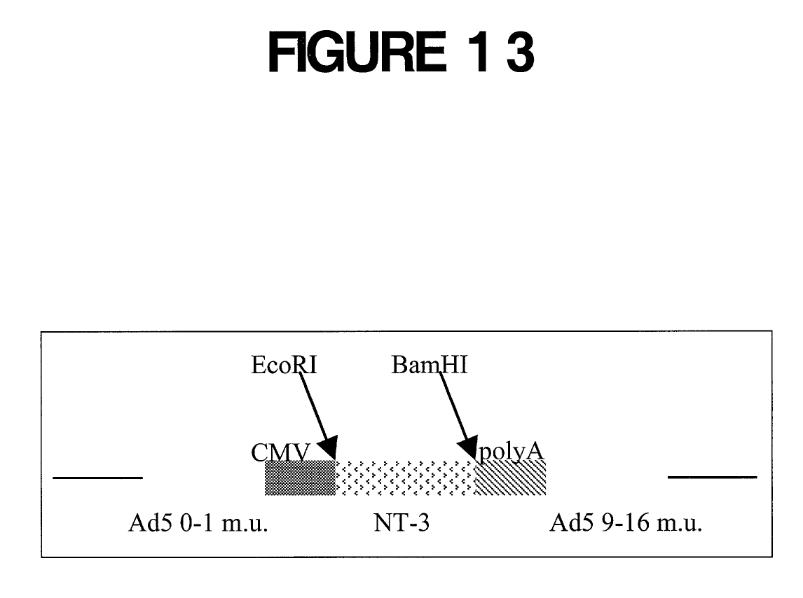

FIG. 14: Fluorescence microscopy analyses of implanted spinal cord section. (A). Syngeneic Sertoli cell survival after three days of implantation. Spinal cord was analyzed for the presence of Sertoli cells by looking for green fluorescent protein expression in the sections. (B). Allogeneic implants tested after three days. (C). Allogeneic implants tested after fifteen days.

FIG. 15. Fluorescence microscopy showing secretion of NT-3 by genetically modified cells in vivo stained blue using a biotinylated antibody to NT-3. Uninjured rat spinal cord was implanted with allogeneic Sertoli cells infected with Ad5GFP/hNT-3. At 15 days post-implantation, sagittal sections of the spinal cord were stained by anti NT-3 Ab (Promega) and analyzed by fluorescence microscopy. (A). Section showing NT-3 expression. (B). Background expression.

FIG. 16: Immune response in injured and uninjured spinal cord: OX42 blue staining of the spinal cord sections to look for macrophage infiltration. (A) Injury alone 3 days post injury, proximal; (B) Injury alone 3 days post injury, distal; (C) Injury plus implantation 3 days; (D) Injury alone 3 days post injury; (E) Injury plus implantation 8 days; (F) Injury alone 8 days post injury; (G) Injury plus implantation 3 days, OX42 blue staining; (H) Injury plus implantation 3 days, green fluorescent protein Sertoli positive cells; (I) Overlay of G and H. showing allogeneic Sertolii cells expressing green fluorescent protein implanted into the spinal cord with a relatively few macrophages stained blue for OX42.

FIG. 17. Graph illustrating results of neurite growth assay. Supernatant obtained from cells infected with Ad5-GFP-NT-3 was used. Cortical neurons grown in culture were treated with varying amounts of the supernatant. Neurite growth medium was completely replaced by different volumes of the conditioned medium (containing NT-3), 1 ml and 2 ml respectively. Control wells contained medium from uninfected cells. Total time of treatment was 19 hours. NT-3 treatment induces modest level of axonal growth in these neurons.

FIG. 18 (A) and (B) illustrates cultured RPE cells from and Callithrix jacchus marmoset stained with hematoxylin-eosin (H & E) showing the pink colored cytoplasmic regions and the darker stained nuclei.

FIG. 19 is a graph representing the cytotoxicity of murine Sertoli (129Ser) and (129Tro) trophoblast cells for allogeneic CD1 lymphocytes. The ELISA measures the release of DNA fragments into the cell medium from cytotoxicity or late stage apoptosis. Cells of the immune system such as cytotoxic T lymphocytes, natural killer cells, and lymphokine-activated killer cells can recognize and destroy target cells. Thus, allogeneic 129 spleenocytes (129SC) were used as a population of allogeneic cells as a positive control. An aliquot of dexamethasone was added to 3 wells (Dex) also as a positive control because it induces apoptosis in T lymphocytes. Syngeneic CD1 spleen cells (CD1 SC) were used as negative control. The mean values of the 129 spleen cells, dexamethasone treated cells, and trophoblast cells were significantly more than the negative control (P<0.05). The data indicate that trophoblast cells will be more successful in defending themselves from attack by the immune system of the host and, therefore, better able to survive allogeneic implantation. This could be particularly important in regions of the body outside of the central nervous system that is partially protected from the immune system.

FIG. 20 contains representative photomicrographs of sections of kidney from Wistar-Furth rats implanted with RPE cells from Lewis rats that have been stained with H & E. Sections shown are 3 days (A) 40× and (B) 200× and 14 days after implantation (C) 40× and (D) 200×. At least a small section of the capsule can be seen in each photograph, and some normal kidney tissue. H & E stains the cytoplasmic portion of cells pink and the nucleus a dark blue.

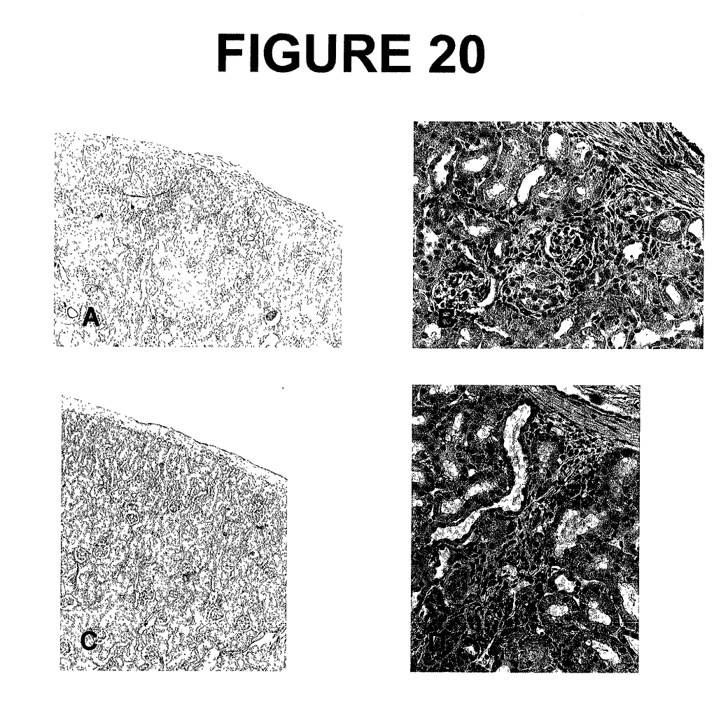

FIG. 21 contains representative photomicrographs of sections of H & E stained kidney from Wistar-Furth rats 14 days after implantation with Wister-Furth (syngeneic) RPE cells (A) 40× and (B) 200×. Sections of kidney from a Wister-Furth rat 3 days after implantation with Sertoli cells from Lewis (allogeneic) rats are shown (C) 40× and (D) 200×.

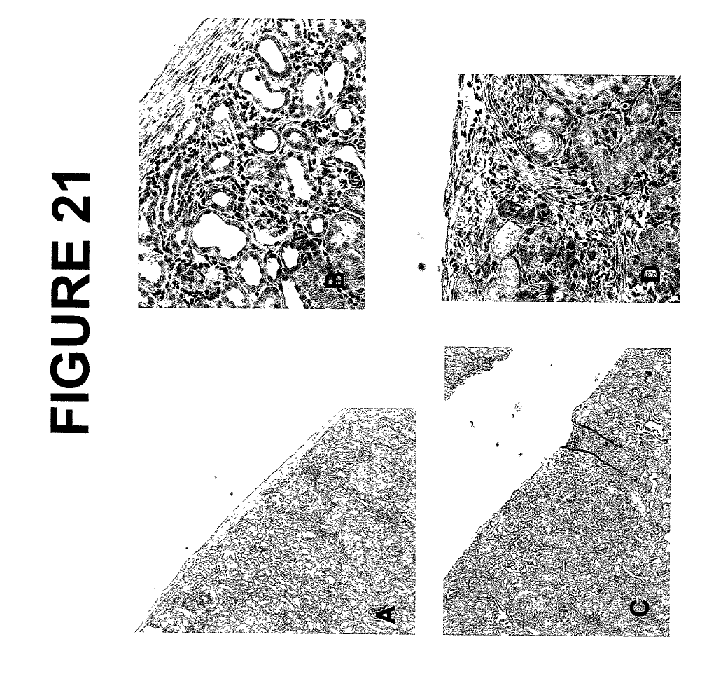

### DETAILED DESCRIPTION OF THE INVENTION

The present invention provides a method for delivery of a biologically active moiety by administering immune-privilege cells that have been genetically modified to express the biologically active moiety. The biologically active moiety is provided in vivo in pharmacologically effective amounts, and is either not naturally expressed by the immune-privileged cells, or is naturally expressed in amounts that are less than required for pharmacologically effectiveness. The biologically active moiety could be a protein, peptide, gene, or the product of a protein such as a neurotransmitter, and could be expressed as a pro-drug that is activated in the body. The administration of the immune-privileged cells can be performed by a variety of methods includeing subcutaneous, intravenous, intraperitoneal, and intramuscular infusion or injection. Additionally, the immune-privileged cells could be implanted in specific sites of the body by a number of surgical procedures. The cells could be adherent to an inert polymeric material that would keep them together at a specific location in the body. The cells could be implanted in a polymeric material that is a liquid that gels upon implantation in the body so that the cells are retained at the site of implantation. The expression of the biologically active moiety could be either intracellular, on the extracellular membrane, or secreted by the cells depending on where the biologically active moiety would be therapeutic. The immune-privileged cells could be freshly isolated cells, or cells that have been cultured, or that have been cultured and then frozen. The immune-privileged cells could be, or derived in culture from, progenitor stem cells of immune-privileged cells. Immune-privileged cells are those that naturally express Fas ligand and that have greater ability to survive allogeneic grafting or implanting with less immunosuppression than other non-immune privileged cells of the body. A listing of cells that express Fas ligand is given in Table 2. The genetic modification of the cells can be performed by the various methods that are known to one of skill in the art. Promoters could be incorporated into the genetic modification of the immune-privileged cells such that the biologically active moiety will be expressed when the promoter is turned-on by the administration, oral or otherwise, of a drug molecule such as tetracycline. The method used for genetic modification could be inserting a transgene with one or more viral vectors. In addition, the genetic modification could be performed by nonviral physical methods such as microinjection, electroporation, lipofection, and chemically-mediated transfection with calcium phosphate or liposomes, and other methods known to one of skill in the art. The pharmacologically effective amount is defined by therapeutic indices or responses appropriate to the disease state or condition that is being treated.

Building on this background knowledge of the immune-privilege inducing function of Fas ligand in naturally occurring cells and tissues, the present invention provides a method for the sustained secretion and delivery of biologically-active proteins and peptides for therapy by implantation of genetically modified allogeneic or xenogeneic cells or tissues derived from immune-privileged sites or tissues. Description of useful embodiments of the invention will be made in detail, which together with the following examples and claims, serve to explain the principles of the invention. This invention is not to be understood as to be limited to the specific examples described, that may vary. The terminology used herein is for descriptive purposes and is not intended to limit the scope of the invention, which will be limited only by the appended claims.

All technical and scientific terms used herein have the same meaning as commonly understood by one of the ordinary skill in the art to which this invention belongs, unless otherwise defined. Methods or materials similar or equivalent to those described here can be used in the practice or testing of the invention, but the preferred methods and materials are now described. All publications mentioned herein are incorporated by reference to describe and disclose specific information for which the reference was cited in connection with.

Advantages and uses of the current invention. Encapsulation of the transgenic immune-privileged cells that are naturally immune privileged would not be required to prevent immune rejection, and long-term survival (months to years) of the cells when implanted in vivo would be predicted. The transgenic cells could be utilized with short-term immunosuppressive therapy to help prevent any immune rejection or inflammatory response. Development of such universal nonautologous cell lines to deliver gene products to different patients will be a successful, efficient, convenient and cost-effective method to deliver the product.

One feature of the present invention is the possibility of site-specific delivery of biosynthetic proteins or peptides by implantation of the immune-privileged cells at the site of interest, or by transfection with cDNA encoding specific adhesion molecules. In some disease states, site-specific rather than or, or in addition to, general systemic delivery of drugs is desirable. For example, the delivery of drugs for treatment of brain tumors or neurodegenerative diseases is hampered by the blood-brain barrier (Domb et al., 1991). Implantation of immune-privileged cells that secrete therapeutic proteins into the brain or central nervous system would enable their stable, continuous and localized delivery and, thus, circumvent the blood-brain barrier.

Another aspect of the invention is the continuous nature of the delivery of the protein or peptide. Intermittent dosing of drugs commonly leads to peaks and troughs in the levels of the drugs and this can be a significant disadvantage. The necessity for the patient to repeatedly take or to be repeatedly administered any drug is inconvenient. In particular, protein drugs tend to be inconvenient, as many of them must be given by injection or infusion. In addition, through oversight or neglect, a significant number of doses of the drug may not be administered and this can result in treatment failures. Slow-release (sustained-release) formulations for many orally available pharmaceutical agents have been developed for these reasons. The present invention will lead to development of a method for continuous or sustained delivery of desired proteins or peptides by the implanted cells.

One feature of the invention is that the use of cells transfected for expression and secretion of particular proteins is one aspect of the whole field of “gene therapy.” Thus, the invention will lead to a novel type of allogeneic or xenogeneic cell-based gene therapy that will require little or no immunosuppression to prevent graft rejection.

Another feature of this invention is its applicability to the production of stably transfected animals possessing naturally occurring immune-privileged sites and/or tissues such as the eye, testes and Sertoli cells that express and secrete specific biomolecules. Immune-privileged cells and tissues of such transgenic donor mammals would be transplanted into recipient mammals or humans and serve as a stable source of sustained delivery of peptides, proteins, and glycoproteins for therapy for specific diseases. The delivery of proteins and other biomolecules could be achieved from the resulting xenografts with little need for chronic immunosuppression. A major advantage of this feature of the invention over the production of transgenic tissues and cells derived from humans for allogeneic grafts is the stable nature of the genetic modification of the cells and tissues of the transgenic animal.

The present invention enables development of cells for sustained, systemic delivery of proteins, glycoproteins, and peptides by the implantation or transplantation into recipient mammals, cells or tissues derived from naturally occurring Fas ligand-expressing allogeneic or xenogeneic cells or tissues, transfected to express and secrete desired proteins or peptides.

In one aspect of the invention, the transfected cells for implantation would be obtained from in vitro transfection in cell culture, or from propagation in vitro of such transfected cells.

In another aspect, the transfected cells or tissues for implantation or transplantation would be obtained from a transgenic animal into which DNA that causes the expression of the desired peptide or protein has been introduced at an embryonic state, or into the ancestor of the animal.

One feature of the invention is a method for systemic delivery of therapeutic proteins, peptides and glycoproteins produced by recombinant technology. The instability and poor absorption of most polypeptide agents in the gastrointestinal tract has necessitated parenteral administration. This invention precludes the need for delivery of these agents by such methods as intravenous injection and enables sustained, systemic delivery of the desired protein or peptide upon implantation of the cells in the appropriate site.

Another feature provided by the invention is a kit containing the transfected or transgenic Fas-ligand expressing cells that secrete the desired peptidic biomolecule for therapy as an article of manufacture.

There are a number of human diseases and conditions in which protein therapy is indicated and for which this invention may be applicable. These include but are not limited to Type 1 and Type 2 diabetes (insulin), Parkinson's disease (tyrosine hydroxylase), neurodegenerative diseases of the central and sympathetic nervous system, (NGF, neurotrophins), anemia (erythropoietin), dwarfism (human growth hormone), diabetes insipidus (vasopressin), and hemophilia (Factors VIII and IX).

For example, hemophilia A is an X chromosome-linked recessive genetic disorder that causes a factor VIII deficiency, and affects 1 to 2 per 10,000 males among all ethnic groups costing approximately $60,000 to $200,000 per patient a year. A long-term preventative therapy would constitute a major advance medically and economically (Dwarki et al., 1995). In its severe form, it is a life-threatening, crippling hemorrhagic disease. Infusions of factor VIII are currently the most widely used therapy, and production of recombinant factor VIII has reduced the number of complications associated with earlier concentrates derived from plasma. Recent data indicates that continuous infusions of factor VIII and other coagulation factor concentrates are superior to repeated bolus injections due to the resultant steady plasma levels obtained that promote hemostasis (Anel et al., 1995). The large size of the gene for factor VIII has increased the difficulty of gene therapy for hemophilia A (Anel et al., 1994).

Hemophilia B is also an X-chromosome linked genetic disorder and is caused by deficiency of factor IX. Hemophilia B affects 1 in 30,000 males. Some promising results have been obtained in animal studies of gene therapy for hemophilia B (Cohen and Kessler, 1995; Dwarki et al., 1995) one in which expression of factor IX was obtained in vivo for more than six months (Dai et al., 1992). In one embodiment of this invention human retinal, Sertoli cells or other immune-privileged cells are isolated, purified, cultured and transfected in vivo with a vector containing a suitable promoter and other elements to express and secrete coagulation factor VIII or IX for use in therapy of hemophilia A or B.

Another use for the current invention is delivery of gene products that can convert an orally administered drug to its active form in a site-specific manner. This type of approach was applied experimentally in vivo using transgenic rat fibroblasts injected stereotaxically and producing a retroviral vector containing the herpes simplex thymidine kinase gene. The drug ganciclovir is converted to its active triphosphate form by the herpes simplex virus thymidine kinase. The producer cells transduced neighboring cancer cells, which were killed by the active form of ganciclovir (Culver et al., 1992). This approach could be applied to the site-specific delivery of other enzymes that activate anticancer agents, for example, the carboxylesterase that can activated the prodrug irinotecan to the potent topoisomerase I inhibitor 7-ethyl-10-hydroxycamptothecin (Potter et al., 1998; Satoh et al., 1994).

Plasmocytes, the type of B cells, which produce and secrete antibodies, have a lifespan of only several days to several weeks and secrete a specific antibody. Genetic modification of other longer-lived cell types to secrete recombinant antibodies in vitro has been demonstrated with bioengineered fibroblasts, hepatocytes and myogenic cells (Noel et al., 1997). The secreted recombinant antibodies had affinities close to that of the parental antibody, with slight differences depending on the cell type. In vivo secretion of recombinant antibodies in the blood stream of mice by myogenic cells lasted for at least four months (Noel et al., 1997). Thus, genetic modification of naturally immune-privileged cells could be used to produce cells that secrete recombinant antibodies. When implanted into humans or other mammals such cells could have applicability for conditions in which long-term antibody therapy is indicated.

In some instances, the use of the genetically modified immune-privileged cells that would be to deliver cell-membrane bound proteins, rather than secreted proteins. For instance, it may be desirable to create a cell with surface receptors that attract and bind toxins or biological molecules in specific tissues. This might be useful in some neurological disorders where an overproduction of a specific neurotransmitter could be ameliorated. Imbalances in protein production in a specific tissue could be regulated by delivery of the cells in a site specific manner. Another use of this approach would be to create cells that have receptors or transporter molecules on their surface enabling a specific function. For instance, an immune-privileged cell naturally expressing Fas ligand could be created that expresses the glucose transporter and an insulin gene complete with glucose response elements (Gros et al., 1997; Newgard, 1998). This would allow the cell to produce insulin in a glucose-regulated fashion as do pancreatic islet cells.

The mechanisms that regulate apoptotic cell death are crucial to a number of biologic processes, including development and normal cell turnover. A number of tissues are characterized by apoptotic cell turnover and express both Fas and Fas ligand (French et al., 1996; Xerri et al., 1997). One embodiment of the present invention uses immune-privileged cells that express both Fas and Fas ligand genetically modified to express recombinant death-inhibitory molecules intracellularly. The death-inhibitory molecules would inhibit the Fas-mediated apoptotic cell death of the immune-privileged cells.

For example, mature primary B cells serve as antigen-presenting cells and could be used for triggering or potentiating immune responses to tumors and viruses, or induction of antigen-specific unresponsiveness. Thus, mature primary B cells could be applicable in the treatment of cancers, viral infections and some metabolic and immunologic disorders (Sutkowski et al., 1994). In a model of somatic cell gene therapy, efficient gene transfer into mature B lymphocytes was achieved with retroviral vectors containing the human adenosine deaminase gene as a marker. The human gene was expressed by B lymphocytes in the spleen of severe-combined immunodeficiency mice (SCID) for at least 3 months (Sutkowski et al., 1994). Mature primary activated B cells express both Fas (Itoh and Naga, 1993) and Fas ligand (Hahne et al., 1996), and thus, they may be susceptible to Fas-mediated apoptosis as well as are capable of inducing Fas-mediated apoptosis.

Fas-mediated apoptosis has been shown to be blocked by the cowpox virus-encoded protein CrmA, an inhibitor of the mammalian cysteine interleukin-1 beta converting enzyme (ICE/caspase-1) (Strasser et al., 1995; Tewari and Dixit, 1995). The family of mammalian ICE-like cysteine proteases are now designated caspases, (cysteinyl aspartate-specific proteinases) because they are cysteine proteases that cleave their substrates following aspartate residues (Nicholson, et al. 1997). Caspases are activated by engagement of the Fas receptor and enable the apoptotic cell death program (Muzio et al., 1997). The bacloviral cell survival protein p35 has been shown to be a broadly-acting inhibitor of the caspases that can inhibit apoptosis in vitro (Miller, 1997; Seshagiri and Miller, 1997), and in vivo (Davidson and Steller, 1998) when expressed intracellularly as a recombinant protein. The expression of cowpox virus-encoded CrmA or bacloviral p35 protein in mature B lymphocytes that naturally express Fas ligand would prevent their own apoptotic cell death and could enable their use in vivo to induce apoptotic cell death in other Fas-expressing cells in an antigen specific fashion for therapy.

The present invention has veterinary applications, for example, in the delivery of protein or peptide drugs to animals. These substances would ordinarily be given to animals orally or by periodic injection. Cellular delivery using the present invention would preclude the necessity of periodic delivery since cells would be administered once to the animal and then would continuously deliver the substance.

The present invention also has industrial applicability in providing hormones, enzymes or drugs to mammals, including humans, in need of sustained doses for extended periods.

Sources of immune-privileged tissues or cells. Expression of Fas ligand is one of the mediators of immune privilege (Bellgrau et al., 1995; Griffith et al., 1995). Fas ligand was originally isolated from a CD4⁺T cell line initially thought to be primarily produced by activated Th2 cells (Suda and Nagata, 1994). However, more recently Fas ligand has been reported to be expressed by other cells including B cells, macrophages, natural killer cells and non-hematopoietic cells including testes, ovary, and salivary gland. Such cell lines could qualify as immune-privileged cells for use in delivery of biomolecules. Expression of Fas ligand reported in various cell lines and tissues is shown in Table 2.

TABLE 2
Cells that express Fas Ligand

| Cell Line | Reference |
|---|---|
| d10S T cell line | (Rouvier et al., 1993) |
| Th1 T cells | (Hahne et al., 1995; Ramsdell et al., 1994; |
| | Suda et al., 1995) |
| CD8⁺ T cells | (Anel et al., 1994; Anel et al., 1995) |
| B cells | (Hahne et al., 1996) |
| Macrophages | (Badley et al., 1996) |
| Natural killer cells | (Arase et al., 1994; Arase et al., 1995; |
| | Montel et al., 1995) |
| Sertoli cells | (Bellgrau et al., 1995) |
| Placenta (trophoblasts, | [Hunt, 1997 #222; Wilson, 1996 #20; |
| decidual cells, endometrial | (Runic et al., 1996)] |
| glandular epithelial & | |
| endothelial cells) | |
| Eye (iris, ciliary body, retina, | (Griffith et al., 1995) |
| corneal epithelium and | |
| endothelium) | |
| Spleen | (Griffith et al., 1995) |
| Paneth cells of the | (Möller et al., 1996) |
| gastrointestinal epithelium | |

Isolation, tissue culture expansion and cryopreservation of immune-privileged cells that are naturally immune privileged. The isolation of primary cells from animal and human tissues and their establishment in culture is common art for most tissues. The steps commonly followed include; enzymatic or physical dissociation of specific cells from a resident tissue, purification of a specific cell type on the basis of (Renjifo et al., 1997; van der Burg et al., 1998) or by using antibodies that recognize cell-specific surface molecules (Geerts et al., 1997; Herbertson and Aubin, 1997) and establishment in culture. Isolation of pure cell types from the eye is well established for lens epithelial cells (Olivero and Furcht, 1993; Wistow et al., 1993), retinal pigment epithelial cells (Martin et al., 1992; Sanders-Sanchez et al., 1990), and retina (Finlay et al., 1996; Wang et al., 1993). Likewise, the purification of Sertoli cells from the testis and their establishment in culture is well described for rat (Cheng, 1990; Hancock et al., 1992; Kelly et al., 1991), hamster (Majumdar et al., 1995), ovine (Monet-Kuntz et al., 1992), porcine (Avallet et al., 1994; Nehar et al., 1997), and primate (Handelsman et al., 1990; Majumdar et al., 1998) cells. Fas ligand expressing cells of the placenta, cytotrophoblasts (Runic et al., 1996; Wilson et al., 1996), are obtainable from elective abortions or term pregnancies and easily purified by density gradient away from contaminating cells types (Bloxam et al., 1997). Cell lines representing some of the immune-privileged tissues are also available (Bourdon et al., 1998; Pognan et al., 1997). Examination of several different immune-privileged cells in culture has shown that Fas ligand synthesis is maintained and even upregulated, supporting the idea that such cells will maintain their immune-privileged status in culture and under transplant conditions (Ortiz-Arduan et al., 1996; Runic et al., 1996; Wilson et al., 1996). Cells that have been genetically modified with the gene of choice are selected to obtain high expressing lines and these are used immediately or stored frozen by conventional methods (Tezel et al., 1997).

Several immune-privileged tissues are readily available from human sources. Placenta can be obtained from elective abortions or from term pregnancies upon delivery. Using techniques described in mouse (Tanaka et al., 1998) trophoblast progenitor stem cells could be derived from human embryos. Eye banks, which collect and store eyes and eye tissues, are common and many people donate this organ. Eye banks are a source for ciliary body, corneal epithelium and endothelium, retina and retinal pigment epithelium. Eye banks also perform blood tests to determine that the tissue is free of disease-causing organisms. Other human tissue can be obtained from patients having elective surgery or at the time of death. Further, many primary human cell types are commercially available as cell lines for research purposes (Clonetics, Walkersville, Md.; American Type Cell Culture, Manassas, Va.).

Genetic modification of immune-privileged cells. The construction of novel cDNAs containing genes of interest mixed and matched with different promoters and other expression controlling elements is well within the everyday technological reach of most laboratories (Ausubel et al., 1994). This is accomplished by first obtaining the gene of interest, which in many cases is available from published sources or in some cases even commercially available. The cDNAs encoding a vast number of proteins that are of interest for production by immune-privileged cells are available and have been for some time. These include but are by no means limited to: human growth factor (hGH) (DeNoto et al., 1981), tyrosine hydroxylase (Grima et al., 1987; O'Malley et al., 1987), coagulation factors VIII (Gitschier et al., 1984) and IX (Kurachi and Davie, 1982), insulin (Bell et al., 1979) and neurotrophin 3 (Jones and Reichardt, 1990) to cite a few. Plasmids are obtained or synthesized fragments are cloned into plasmids, which are then tailored to meet the needs of the project.

Promoter composition is a major consideration in designing a transgene. Gene expression is controlled at several different levels but transcriptional initiation is a critical event in determining how much of a gene will be produced. Transcription depends on specific promoter and enhancer sequences within the DNA and is influenced by cellular factors that interact with these elements. Hybrid promoters can be constructed which utilize several different bacterial and/or viral elements to achieve the desired level of cDNA expression (Gage et al., 1997). However, it has been found that some viral promoters which are very strong in vitro downregulate in vivo (Dwarki et al., 1995; Gage et al., 1997; Hurwitz et al., 1997; Palmer et al., 1991; Ramesh et al., 1993; St Louis and Verma, 1988; Vogt et al., 1994). For this reason, the use of cellular promoters derived from housekeeping genes or of tissue specific promoters that are active only in specific tissues are of great value (Gage et al., 1997). There are also promoters that respond to the presence of substances that are present in tissues, such as cytokines (Gage et al., 1997) or that respond to substances that can be given to the animal such as tetracycline. Retroviral vectors, carrying tetracycline responsive elements, are commercially available (Clontech Laboratories, South San Francisco, Calif.). The use of such promoters confers the ability to express genes in specific cells or to control expression by exogenous means.

There are many possible means to introduce genetic material into host cells. Any virus that can express new genetic material in host cells can be used including SV40, herpes virus, adenovirus, adeno-associated virus, and human papilloma virus. Some viruses have the advantage that they will integrate into the host genome in the absence of cell replication. These include adeno-associated viruses (Freese et al., 1997; Kaplitt et al., 1994) and lentiviruses (Miyoshi et al., 1997; Naldini et al., 1996). Replication deficient retroviruses have been a preferred method, have been widely used and are commercially available (Clontech, South San Francisco, Calif.). Chemical transfection methods can also be used, such as calcium phosphate coprecipitation or DEAE-dextran. DNA can also be introduced through electroporation, by microinjection and by liposome delivery. These methods and their advantages are reviewed in Gershon et al (Gershon et al., 1997).

Experiments in the specific transfection of immune-privileged cells and tissues in order to express recombinant proteins demonstrate the feasibility of producing proteins in cells that are naturally immune privileged. Bennett and co-workers achieved adenovirus vector-mediated in vivo gene transfer in the adult (post-mitotic) murine retina using the cytomegalovirus (CMV)-promoted Escherichia coli reporter gene, lacZ, by injection into the subretinal space of the peripheral retina (Bennett et al., 1994). The study was undertaken to establish methods for introduction of therapeutic genes into adult mammalian retina towards development of new treatments for currently untreatable, inherited retinal diseases. There was no decrease in lacZ expression after 95 days, although there was a decrease in the intensity of the staining. Many cells of the outer retina, including the photoreceptors expressed lacZ and some cells transversing the neural retina occasionally expressed lacZ. Other laboratories have reported achieving gene transfer into murine retinal cells mediated by adenovirus (Jomary et al., 1994), and by retrovirus (Dunaief et al., 1995; Schubert et al., 1998). In addition, the successful transfection of human retinal pigment epithelium by electroporation has been reported (Williams et al., 1994). Transfection of retinal cells of the rat can be achieved from liposomes in eye drops applied topically to the ocular surface (Matsuo et al., 1996). Many other immune-privileged cell types have also been successfully transfected (Chaudhary et al., 1996; Ducray et al., 1998; Franklin et al., 1991; Jacquemin et al., 1996; Johnson et al., 1997).

Transgenic Animals. In a preferred embodiment of this invention immune-privileged cells and tissues expressing the desired protein or biomolecule are obtained from transgenic animals. Transgenic animals are produced by transfections of the germ cells (usually oocytes) rather than the somatic cells that are the targets of gene therapy efforts. There are many routes into the germ-line cells, but by far the most widely used is the microinjection of foreign genes into one of the two pronuclei of a fertilized oocyte. The first transgenic mice produced by the microinjection technique were generated in 1980 (Gordon et al., 1980). Since then hundreds of transgenic mice lines have been created (Gordon et al., 1980; Jaenisch, 1988; Mountz et al., 1990; Palmiter and Brinster, 1986).

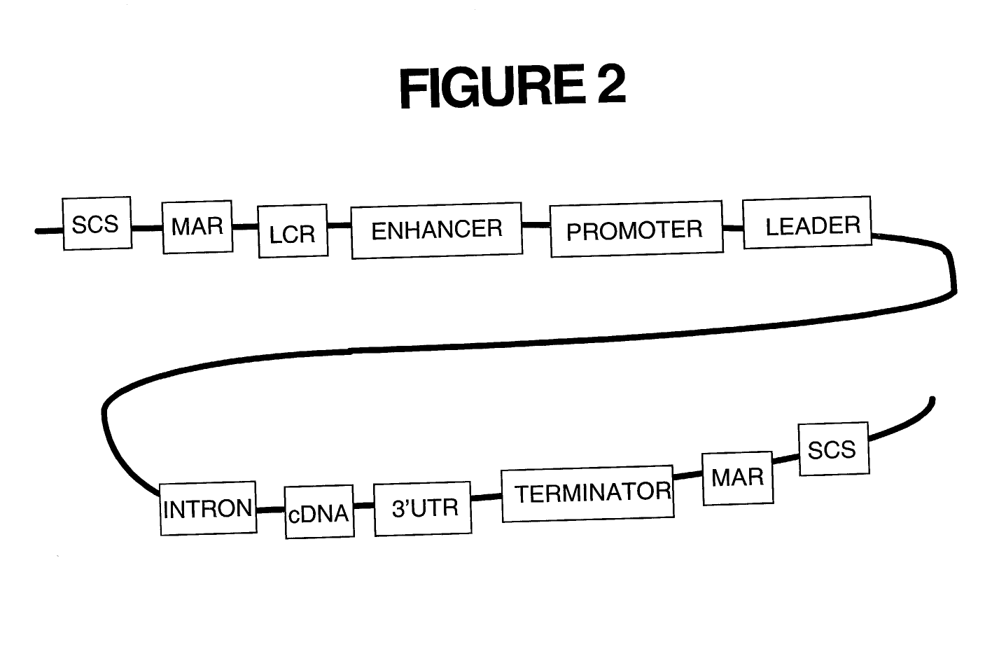

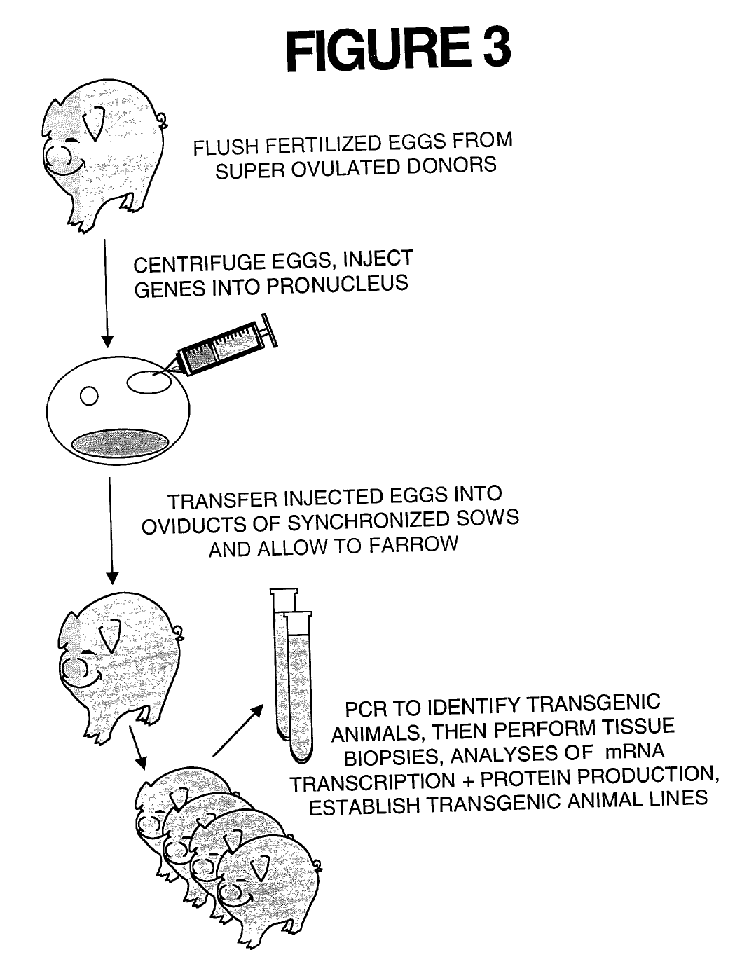

Transgenic animals can be created by methods known to one of ordinary skill in the art, and can be found in numerous guides and laboratory manuals such as those by J. D. Mountz, et al. (Mountz et al., 1990), C. A. Pinkert, Ed. (Pinkert, 1994), and D. Murphy and D. A. Carter (Murphy and Carter, 1993). These manuals provide information relevant to transfection of goats, sheep, cattle, swine, poultry, fish, rats, rabbits, and mice (Ausubel et al., 1994; Barr and Leiden, 1991; Bouck and DiMayorca, 1979; Chen and Okayama, 1987; Dhawan et al., 1991; Mountz et al., 1990; Murphy and Carter, 1993; Pinkert, 1994; Seldon et al., 1986). A general diagram of a transgene is presented in FIG. 2, and the application of xenogeneic cells for therapy is depicted in FIG. 3, adapted from the manual edited by C. A. Pinkert (Pinkert, 1994). In addition to myriad transgenic rats and mice, there are transgenic rabbits (Dunn et al., 1995; Duverger et al., 1996), cows (Cibelli et al., 1998), sheep (Damak et al., 1996; Harris et al., 1997; Schnieke et al., 1997) and pigs (Li et al., 1998; Piedrahita et al., 1997; Zaidi et al., 1998). Further, many patents have been awarded covering inventions involving transgenic animals; for the production of hormones (Evans et al., 1989), antibody (Lonberg and Kay, 1997) and production of proteins in milk (Archibald et al., 1997; Deboer et al., 1998).

The microinjection technique requires three separate steps (Mountz et al., 1990; Murphy and Carter, 1993; Pinkert, 1994). The first is the production and isolation of fertilized single-cell embryos. The second step is injection the desired transgene into the pronucleus, which will become integrated, probably during chromosomal repair. The third step is the implantation of up to 30 injected viable embryos into the oviduct of a pseudopregnant recipient female. As a result of integration at the one-cell embryo stage, the foreign gene potentially occurs in every cell of the animal when it is bom. Gene transfer can also be accomplished by retroviral infection of early embryos or transferring the transgene into embryonal stem cells followed by the introduction of the stem cells into blastocysts. Transgenic methods are standard and many academic institutions have transgenic facilities that create transgenic animals on a contract basis. Further, commercial services that produce transgenic animals are also widely available in the US and Europe.

One-cell embryos can be obtained through an all in vitro protocol. This method includes the following steps: collection of ovaries from females at any physiological stage, in vitro maturation of oocytes isolated from the ovaries, and in vitro fertilization of the oocytes. The availability of embryos is considerably increased with this procedure. The method has been defined and used in cows, sheep and goats. After gene microinjection, bovine embryos can be cultured up to the blastocyst stage. Only the embryos surviving the manipulation reach this stage. The blastocysts can then be transferred into pseudopregnant females and transgenic animals will be produced. Detection of the transgene in a few cells explanted from the blastocytes can be performed using the PCR technique. Currently there is no way to control in most cases the number of copies of a cDNA that incorporate into the host genome or the insertion site. Thus, some animals will exhibit low expression of the transgene due to either copy number or to insertion site. Fortunately, it is possible to screen for high expressing lines and also to determine copy number and germ line transmissability.

In one embodiment of the present invention transgenic pigs and rats are produced and bred with naturally occurring immune-privileged cells such as Sertoli cells that express and secrete human growth hormone (hGH). Towards this goal, a vector containing the gene for hGH and other necessary elements such as promoter, enhancer, introns, etc. (see FIG. 2) is created. Fertilized single-cell rat embryos are isolated and injected with the gene of interest. Thirty injected viable embryos are implanted back into the oviduct of each pseudopregnant recipient female.

The Sertoli cells are then isolated, purified and characterized for expression and secretion of hGH using immunohistochemistry and ELISA. One part of this process involves transplantation into an animal in order to assess the in vivo stability of the transgene and the cells. After complete characterization, the transgenic cells are used for implantation in the kidney capsule of dwarf rats, a model for hGH deficiency. Regular monitoring of the plasma levels of the protein is performed in order to determine the safety and efficacy of the therapy and to adjust the dose of cells.

One aspect of the present invention would be a pharmaceutical composition comprised of the transfected or transgenic naturally immune-privileged cells in a kit with an pharmaceutically acceptable carrier including any and all solvents, dispersion media, coatings, antibacterial and antifungal agents, isotonic agents and the like. The use of such media and agents is well-known in the art. The present invention further contemplates a pharmaceutical composition comprising transgenic immunr-privileged cells that secrete desired proteins or peptides for therapy.

In another formulation, genetically modified, immune-privileged cells can be cultured on substrates, like collagen or synthetic skin that could be applied externally to wounds, to the skin or to incision sites following surgery. Further the cells can be incorporated into inert biological or polymeric matrices to retain organization in a specific graft site.

The immune-privileged cells could be immortalized that would allow them to be continuously cultured for long periods of time with little change (Bartek et al., 1991; Hayward et al., 1995). Immortalized cells will divide indefinitely, but are non-tumorogenic. One method of immortalization of cells is transfection with a virus, such as a recombinant retrovirus, carrying the gene for simian virus 40 large tumor antigen (Bartek et al., 1991; Hayward et al., 1995). Another alternative is the use of progenitor stem cells such as trophoblast stem cells (Tanaka et al., 1998).

The following specific examples are given as illustrative embodiments of the invention described more generally above. The invention is exemplified by preferred embodiments in which genetically modified immune-privileged cells are created by different methods including transfection of cells in culture or isolation of cells from transgenic animals. The first example describes the creation of a retroviral vector that carries the human neurotrophin 3 (NT3) gene in a retroviral expression vector.

The second example describes the creation of genetically modified porcine pigment epithelial (PRPE) cells. The cells are created by transfection with the retroviral vectors created in the first example and produce NT3 in vitro and in vivo and exhibit immune-privileged status by virtue of the continuous expression of Fas ligand.

In the third example, genetically modified rat Sertoli cells that are naturally immune privileged are created using the retroviral expression vector from Example 1. The cells also express human NT3 and retain their Fas ligand expression.

The fourth example describes the implantation of the pRPE and the rat Sertoli cells in an animal model of spinal cord injury. NT3 is known to reduce the effects of spinal cord injury, thus the presence of genetically modified, NT3-producing cells is assayed by behavioral and immunocytochemical means.

In the fifth example, transgenic rats are created which express human growth hormone (hGH) expressed specifically in their Sertoli cells. This is accomplished by means of a tissue specific promoter (Reventos et al., 1993). The transgenic animal is created by inserting a cDNA construct into the rat genome. This construct carries the hGH gene controlled by the androgen binding protein (ABP) promoter. Since this promoter is only active in Sertoli cells, the hGH will be specifically expressed in Sertoli cells. This has two advantages, the first is that the growth biology of the transgenic animal will be minimally affected since the protein is expressed in a limited fashion, rather than in all tissues. The second advantage is that cell specific promoters are known to be more active in implanted cells than viral promoters, which are frequently down-regulated in vivo (Palmer et al., 1991).

The implantation of Sertoli cells producing hGH into a dwarf rat model is described in example 6.

Creation of human cells, genetically modified to produce tyrosine hydroxylase and amino acid decarboxylase (TH/AADC) is described in example 7. A retroviral expression vector is used to create an immune privileged cell that expresses the TH/MDC cassette under the control of the RPE-cell specific promoter, RPE65 (Nicoletti et al., 1998). Cell-specific promoters are more effective in maintaining gene expression in implanted cells (Palmer et al., 1991).

The TH/AADC-producing cells are implanted into a rat model of Parkinson's disease in example 8. The production of dopamine by cells in situ is assessed by behavioral assay and by immunocytochemical methods.

Allogeneic Sertoli cells secreting neurotrophin-3 are implanted into the rat contusion model of spinal cord injury in example 9. The contusion model creates injuries that are more similar to clinically observed spinal cord injuries than other models. This example shows the utility of immune-privileged cells in delivery of a biologically active moiety that is not naturally secreted by the cells in a disease state, and demonstrates the ability to deliver biologically active protein into the central nervous system where is normally difficult to obtain therapeutic concentrations of drugs.

In example 10 the isolation and culture of RPE and Sertoli cells from Callithrix jacchus marmoset is described. The C. jacchus marmoset experimental allergic encephalomyelitis (EAE) model for multiple sclerosis model (Genain and Hauser, 1997) has greater similarity to human MS than rodent models of acute EAE and is an ideal system to test future gene-based therapeutic strategies, because of evolutionary similarity between C. jacchus and humans. Intraventricular delivery of II-10 and nerve growth factor, and other proteins of potential therapeutic use are to be evaluated using genetically immune-privileged marmoset cells.

In vitro comparative assays for the cytotoxity or apoptosis of immune privileged cells towards allogeneic lymphocytes and assays for their ability to suppress mixed lymphocyte reactions and to cause minimal proliferation of allogeneic spleen cells are described in example 11. These assays reveal differences in the biological activity of two types of immune-privileged cells, Sertoli cells and trophoblast cells, that have relevance to the ability of the cells to survive as allografts. The trophoblast cells induced significantly more cell death in allogeneic spleen cells than did syngeneic spleen cells whereas this was not true for the Sertoli cells. Therefore the trophoblast cells would be more able to ward off an attack by the cells of the immune system of an allogeneic host than the Sertoli cells.

Comparative implants of immune privileged cells into the kidney capsule of rats are described in example 12. Analyses of the survival of different immune privileged cells at various sites in the body is helpful in determining the best type to use in development of cellular protein drug delivery vehicles.

### EXAMPLE 1

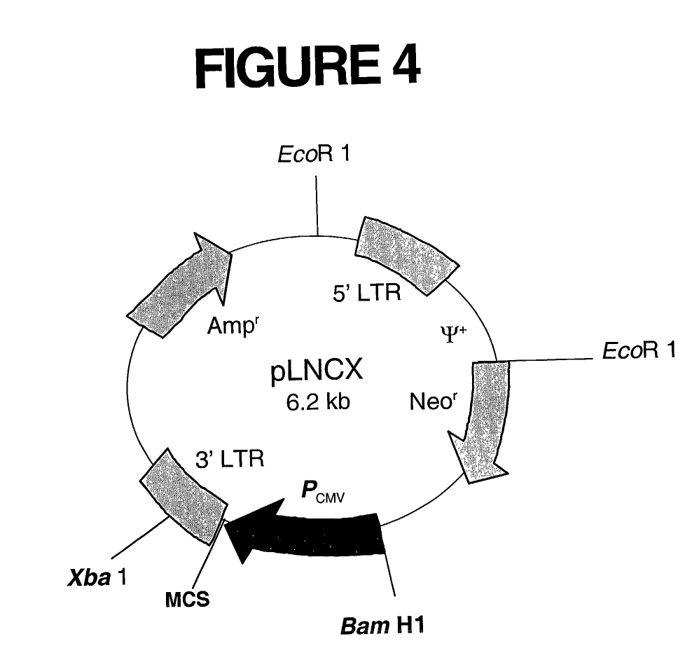

Construction of retroviral expression vector (vNT3LNCX) carrying human neurotrophin 3 (hNT3) gene. The vNT3LNCX retroviral vector is constructed by first inserting NT3 cDNA into the retroviral vector pLNCX. Plasmid pLNCX (Clontech, South San Francisco, Calif.) is derived from the Moloney murine leukemia virus (MoMuLV) and is designed for retroviral gene delivery and expression (FIG. 4). pLNCX contains the extended viral packaging signal Psi (ψ), and the neomycin resistance gene (Neo^{r}) a selectable marker. Expression of Neo^{r} confers resistance to neomycin which allows selection of cells expressing the plasmid (Southern and Berg, 1982). In pLNCX, neo^{r} gene expression is under control of the 5′ viral LTR while the human cytomegalovirus (CMV) promoter controls the expression of the inserted gene (in this case neurotrophin 3, NT3). The CMV promoter is typically a stronger promoter than the viral LTR promoter, which leads to a robust expression of the gene of interest. The retroviral genes required for retroviral replication have been deleted from the pLNCX plasmid which is thus replication defective.

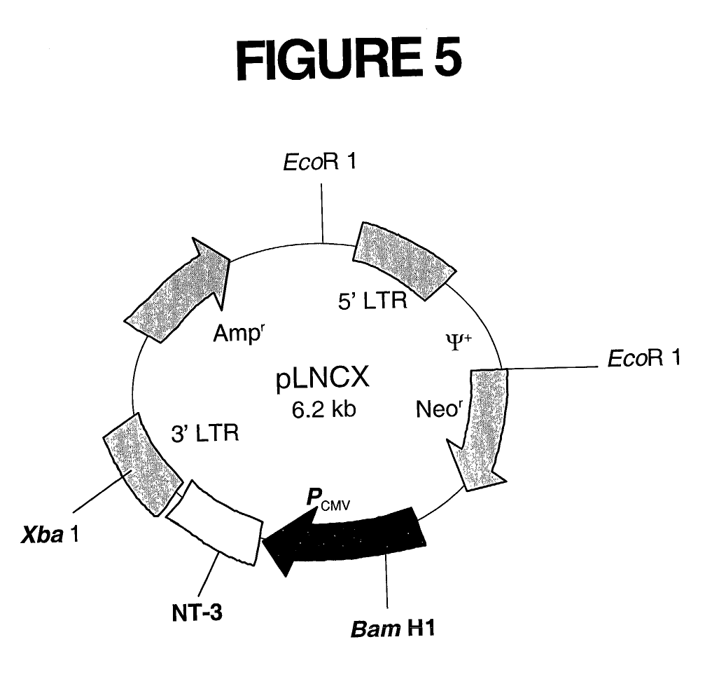

The NT3 sequence is released from the plasmid (FIG. 5) by digestion with the Hind III restriction endonuclease. This yields a 908 bp fragment containing the NT3 cDNA (Senut et al., 1995). To insert the NT3 cDNA into the pLNCX retroviral backbone, the pLNCX plasmid is digested with Hind III restriction endonuclease and treated with phosphatase following standard molecular biological techniques (Ausubel et al., 1994). The NT3 cDNA is then ligated into pLNCX using DNA ligase. Following ligation, DH5α E.coli cells are transformed with the resultant plasmid. Individual transformants are grown, harvested and analyzed by restriction endonuclease mapping to identify bacterial clones with the desired plasmid in the correct orientation. Several such transformants, which yield the predicted fragments upon endonuclease digestion (FIG. 5) are chosen and the orientation and junction structure is confirmed by cDNA sequencing. Thus, in the pNT3LNCX plasmid the NT3 cDNA sequence is immediately downstream from the CMV immediate early promoter in the pLNCX sequence. Two different mRNA transcripts are produced in cells transduced with this virus; one under control of the LTR promoter that contains the Neo^{r} product and another under control of the CMV promoter that produces the gene of interest, in this case, NT3 (FIG. 5).

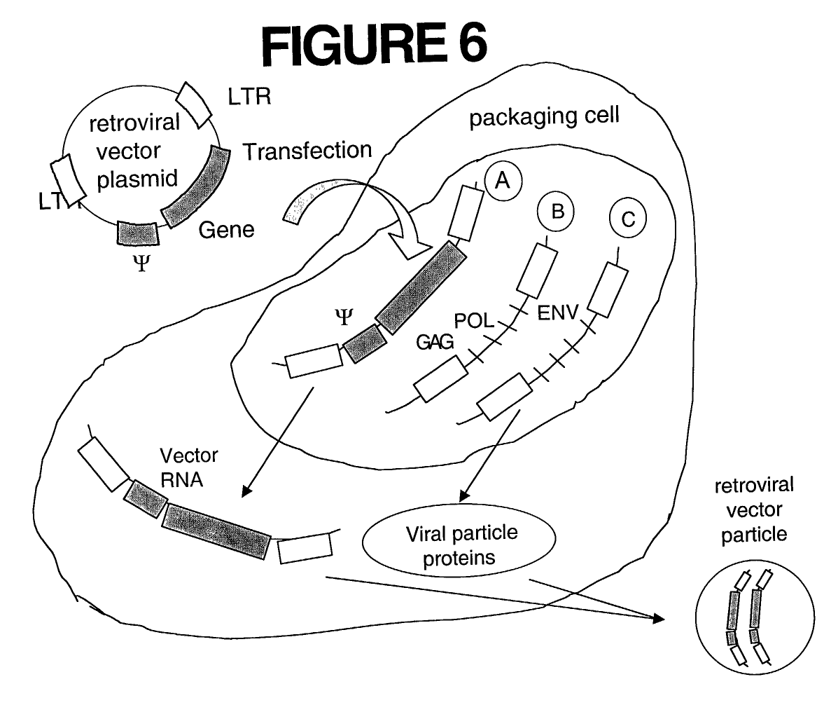

Retroviral plasmid pNT3LNCX is isolated and purified from the bacteria by standard techniques and transfected into the packaging cell line, PT67 (Clontech User Manual, see FIG. 6). The PT67 cell line contains the structural genes necessary for particle formation and replication, gag, pol and env, but not the ψ packaging signal. Introduction of a retroviral vector containing the ψ signal, transcription and processing elements and the gene of interest results in the production of replication incompetent virus. These retroviral particles can infect target cells but cannot replicate within the target cells since they lack the viral structural genes (Clontech User Manual, PT3132-1). Separate introduction of the structural genes into PT67 minimizes the possibility of the production of replication-competent virus due to recombination events during cell proliferation (Miller and Chen, 1996; Morgenstern and Land, 1990). Packaging cells are plated at 5-7×10⁵ cells per 100 mm² plate, 12 to 24 hours before transfection and fed 1-2 hours prior to transfection. Cells are transfected by the calcium phosphate co-precipitation method (Richmond et al., 1988; Wigler et al., 1977). Each plate of cells is transfected with 10-15 μg of plasmid DNA. The virus produced from the PT67 cell line bears protein 10A1, can enter cells by either of two different surface receptors and has a broad host range (Miller, 1996; Miller and Miller, 1994). Stable virus-producing cell lines are selected by maintaining the cells in selection medium, containing G418 (0.5 mg/ml) for 1 week following transfection. Viral titer is determined and individual high titer clones are selected following screening of 20-50 clones. High titer clones are then expanded, and maintained as frozen stocks. Cells are grown in the absence of G418 for viral production. Supernatant culture medium from confluent cultures of high viral titer cells is collected, filtered to remove remaining cells (0.45 μ filter, cellulose acetate or polysulfonic low protein binding) and stored at −80° C. or used immediately. Aliquots are frozen depending on viral titer since repeated freezing and thawing reduces the titer.

### EXAMPLER 2

Creation of genetically modified immune-privileged cells from porcine retinal pigment epithelium (RPE) producing human neurotrophin 3 (hNT3). Isolation, purification, tissue culture expansion and cryopreservation of porcine RPE cells. Porcine retinal pigment epithelial (PRPE) cells are isolated from porcine eyes obtained from a local abattoir. Eyes are rinsed in phosphate buffered saline (PBS) containing antibiotics (100U/ml penicillan and streptomycin). The anterior segment, retina and vitreous humor are removed. Eye cups are incubated at 37° C. in 5% CO₂ with 0.3% trypsin in Ca⁺⁺/Mg⁺⁺ free PBS, containing 0.5 mM ethylene diamine tetraacetic acid (EDTA) for 45 minutes (Esser et al., 1997; Jaffe et al., 1990). The retinal pigment epithelium is dislodged from Bruch's membrane and cells are gently triturated to achieve a single cell suspension which is plated in Dulbecco's modified Eagle's medium, supplemented with 15% fetal calf serum, 50 μg/ml gentamicin, and 2.5 μg/ml amphotericin. Retinal pigment epithelial origin and maintenance of phenotype is confirmed by cytokeratin immunocytochemical analysis (Esser et al., 1997). This isolation technique yields pure RPE cultures, free of contaminating choroidal cells (Jaffe et al., 1990). Media are changed twice weekly and cells are grown to confluency on 75-cm² flasks. Subsequent passaging of cells is performed by trypsinization using standard protocols.

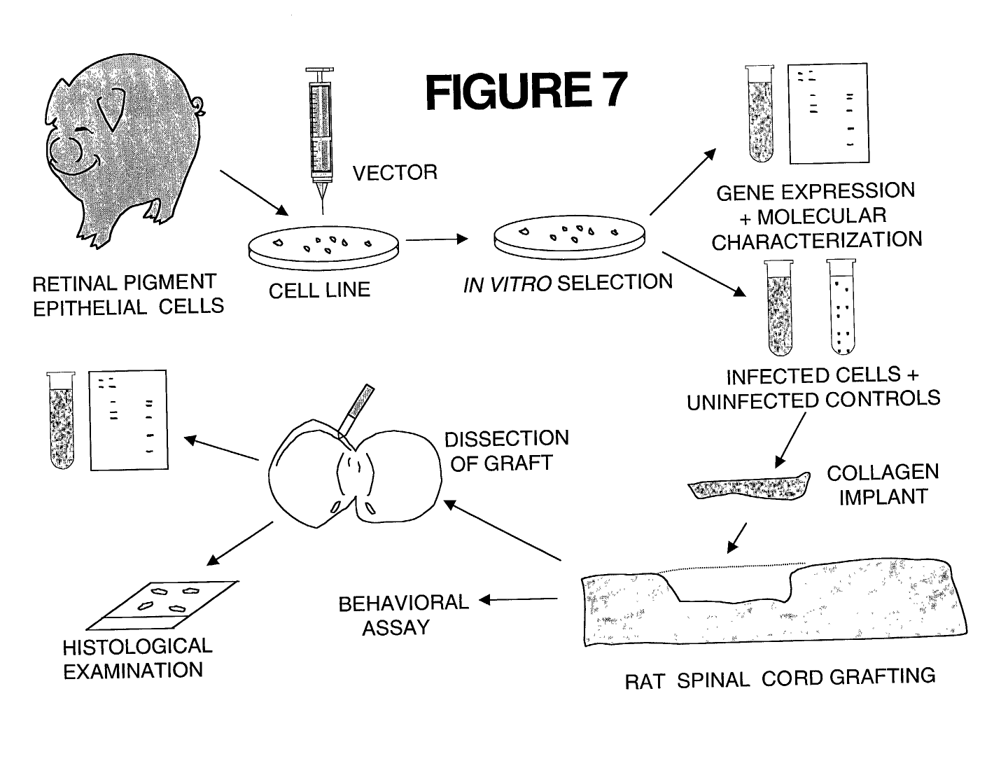

Viral infection of pRPE Cells. To achieve viral infection (see FIG. 7), pRPE cells are plated 12-18 hours prior to infection at a cell density of 3-5×10⁵ cells per 100 mm plate in complete culture medium containing heat-inactivated serum, which lacks complement that could inactivate retrovirus (Mochii et al., 1998). Filtered virus-containing medium obtained from the packaging cells (see above) is placed on the cells and polybrene is added to the culture to a final concentration of 4 μg/ml. Complete culture medium is replaced after 24 hours. In general, half-maximal infection takes place after 5-6 hours and maximal infection takes place after 24 hours. Viral reverse transcription and integration takes place between 24 and 36 hours following infection. Expression of the transgene can be observed as early as 24 hours postinfection and usually reaches a maximum at about 48 hours. At this point cells are subjected to selection with G41 8 (1 mg/ml). The cells are also examined by PCR to confirm that no wild-type virus is present in the culture supernatant. Selected cells are grown to 80% confluence in the presence of 0.5 mg/ml G418 then passed by trypsinization and aliquots are frozen by standard techniques (Tezel et al., 1997).

Determination of FasL expression and transgene expression in virally transduced pRPE. Virally transfected pRPE cells are characterized with regard to maintenance of phenotype and transgene expression (FIG. 5). The primary phenotypic characteristic of pRPE cells that is required for the present experiment/invention is the continued expression of Fas ligand (FasL) by the cultured pRPE cells. Infected cells are therefore grown in culture and scored for the expression of FasL. During routine passaging of cells, an aliquot of cells in solution is plated onto a collagen-coated chamber slide (Costar) and cells are allowed to attach for 12 to 24 hours. Cells are then stained for the surface expression of FasL. Briefly, the cells are fixed in 4% paraformaldehyde in PBS, pH 7.4 for 15 minutes on ice then rinsed with cold PBS and blocked by incubation with 10% normal goat serum (NGS) in PBS for 30 minutes. Cells are then incubated with antiFas ligand antibody (Calbiochem, San Diego, Calif. #PC78) at a concentration of 10 μg/ml in 10% NGS in PBS for 1 hour at room temperature. Following incubation with the primary antibody the slides are rinsed in 10% NGS in PBS then incubated with a biotinylated goat anti-rabbit antibody (Vector Labs, Burlingame, Calif.) for one hour at room temperature. The Vectastain Elite kit (Vector Labs # PK-61 01) is used to develop the color reaction. Samples are examined under magnification and scored for the number of stained cells per total cell population. In general, cells in culture maintain or upregulate expression of Fas ligand (Ortiz-Arduan et al., 1996; Runic et al., 1996; Wilson et al., 1996).

Cell surface FasL is released into the culture medium by the result of cleavage by metalloproteinase (Kayagaki et al., 1995). FasL presence in the culture medium is determined by ELISA (Kayagaki et al., 1995).

Transgene (NT3) expression is determined by quantitative reverse transcriptase-polymerase chain reaction (RT-PCR) of pRPE in culture and by biological assay (Senut et al., 1995). RNA is extracted and purified as described (Ausubel et al., 1994). Reverse transcription (RT) is performed using 25-50 ng of total RNA in a reaction mixture containing the RNA, PCR buffer, (10 mM Tris-HCl, pH 8.3, 50 mM KCI), 1 mM dNTP (Boehringer Mannheim, Indianapolis, Ind.), 2.5 nM MgCl₂, 20 U RNAsin (Promega, Madison, Wis.), 100 pM random hexamers (Boehringer Mannheim) and 12.5 U AMV reverse transcriptase (Promega) in a reaction volume of 20 μl. The reaction mixture is incubated in a Perkin-Elmer Thermal Cycler for 75 minutes at 42° C. and 10 minutes at 95° C. RT cDNAs are used immediately for PCR amplification. Two sets of primers are used for PCR amplification: human NT3 (hNT3) (Senut et al., 1995) and porcine β actin (Li et al., 1997). Primer sequences are as follows: hNT3, 5′ primer, (SEQ ID NO: 1), 3′ primer, (SEQ ID NO: 2), 123 bp product, and porcine β actin, 5′ primer, (SEQ ID NO: 3). PCR amplification is performed in an 80 μl reaction mixture containing the RT product, PCR buffer, 1.75 mM MgCl₂, 0.5 μg of each primer, 2.5 U Taq polymerase (Perkin-Elmer Cetus, Norwalk, Conn.) and 2 μCi of (³²P)dCTP, in a Perkin-Elmer Cetus Thermocycler as described (Senut et al., 1995). Samples are separated by electrophoresis (PAGE), dried and exposed on x-ray film, then developed. The radioactive signal is quantified by measuring the cpm from the membrane by scintillation counting. NT3 values are normalized to the control gene.

NT3 protein production by cells in culture is measured by ELISA (Sadick et al., 1997; Smith et al., 1996) and biological assay as described (Senut et al., 1995). For the biological assay, sympathetic chain and dorsal root ganglia are dissected from embryonic day 9 (ED 9) chick embryos and dissociated by trypsinization. Cells are counted and plated at a concentration of 2000 cells/well in a 24 well plate coated with 5 μg/ml laminin. Cells are maintained in culture for 48 hours in N2 medium (Bottenstein and Sato, 1980) ] alone or containing recombinant hNT3 (Promega, Madison, Wis.), murine nerve growth factor (mNGF), or supernatant from the virally transfected (NT3) and control pRPE cells. Cell survival and neurite extension is assessed by viewing under magnification after 48 hours. In this way it is possible to obtain a stable transfectant population of FasL positive pRPE that are producing NT3.

### EXAMPLE 3

Creation of genetically modified rat Sertoli cells producing human NT3 (hNT3). Cell isolation, purification, tissue culture expansion and cryopreservation of rat Sertoli cells. Sertoli cells are isolated from euthanized Sprague-Dawley rats as previously described (Cameron et al., 1987; Korbutt et al., 1997). Testes are removed from the animal, skinned and collected in cold PBS, minced into 1 mm pieces and then subjected to sequential enzymatic treatment at 37° C. using first 0.1% collagenase for 10 minutes (Sigma, St Louis, Mo., type V). This digest is washed 3 times in Ca⁺⁺/Mg⁺⁺ free PBS (CMF-PBS) containing 1 mM EDTA and 0.5% bovine serum albumin (Sigma), then digested for 10 minutes at 37° C. with trypsin (0.25 μg/ml) and DNAse (4 μg/ml, Boehringer Mannheim, Indianapolis, Ind.) in CMF-PBS. The resultant cell suspension is suspended in Ham's F10 medium containing 10 mM glucose, 2 mM 1-glutamine, 50 μM isobutylmethylxanthine, 100 U/ml penicillan, 100 μg/ml streptomycin, and 5% Lewis rat serum. The cell suspension is passed through a 500 μm mesh, plated onto tissue culture wells (Costar/Corning, Acton, Mass.) and incubated at 39° C. in 5%CO₂/95% air for 48 hours. Each culture well is then treated with sterile 20 mM Tris-HCl buffer for 2.5 minutes with agitation, which detaches contaminating germ cells (Galdieri et al., 1981). Sertoli cells are expanded in culture, aliquots are used immediately for retroviral infection or frozen by standard cell biological techniques and stored for later use (Freshney, 1994; Selawry et al., 1996).

Viral infection of rat Sertoli cells. To achieve viral infection (FIG. 5), Sertoli cells are plated 12-18 hours prior to infection at a cell density of 3-5×10⁵ cells per 100 mm plate in complete culture medium containing heat-inactivated serum, which lacks complement that could inactivate retrovirus (Mochii et al., 1998). Filtered virus-containing medium obtained from the packaging cells (see above) is placed on the cells and polybrene is added to the culture to a final concentration of 4 μg/ml. Complete culture medium is replaced after 24 hours. In general, half-maximal infection takes place after 5-6 hours and maximal infection takes place after 24 hours. Viral reverse transcription and integration takes place between 24 and 36 hours following infection. Expression of the transgene can be observed as early as 24 hours post infection and usually reaches a maximum at about 48 hours. At this point cells are subjected to selection with G418 (1 mg/ml). The cells are also examined by PCR to confirm that no wild-type virus is present in the culture supematant. Selected cells are grown to 80% confluence in the presence of 0.5 mg/ml G418 then passed by trypsinization and aliquots are frozen by standard techniques (Freshney, 1994; Selawry et al., 1996).

Determination of FasL expression and transgene expression in virally transduced rat Sertoli cells. Virally transfected Sertoli cells are characterized with regard to maintenance of phenotype and transgene expression (FIG. 6). The primary phenotypic characteristic of Sertoli cells that is required for the present experimenvinvention is the continued expression of FasL. Infected cells are therefore grown in culture and scored for the expression of FasL. During routine passaging of cells, an aliquot of cells in solution is plated onto a collagen coated chamber slide (Costar/Corning, Acton, Mass.) and cells are allowed to attach for 12 to 24 hours. Cells are then stained for the surface expression of FasL. Briefly, the cells are fixed in 4% paraformaldehyde in PBS, pH 7.4 for 15 minutes on ice, then rinsed with cold PBS and blocked by incubation with 10% normal goat serum in PBS for 30 minutes. Cells are then incubated with antiFasL antibody (Calbiochem, San Diego, Calif. #PC78) at a concentration of 10 μg/ml in 10% normal goat serum (NGS) in PBS for 1 hour at room temperature. Following incubation with the primary antibody the slides are rinsed in 10% NGS in PBS then incubated with a biotinylated goat anti-rabbit antibody (Vector Labs, Burlingame, Calif. #BA 1000) for one hour at room temperature. The Vectastain Elite kit (Vector Labs # PK-6101) is used to develop the color reaction. Samples are examined under magnification and scored for the number of stained cells per total cell population. In general, cells in culture maintain or upregulate expression of Fas ligand (Ortiz-Arduan et al., 1996; Runic et al., 1996; Wilson et al., 1996).

Cell surface FasL is released into the culture medium by the result of cleavage by metalloproteinase (Kayagaki et al., 1995). FasL is released from the cell surface by the action of metalloproteinase, the presence of FasL in the culture medium is determined by ELISA (Kayagaki et al., 1995).

Transgene (NT3) expression is determined by quantitative reverse transcriptase-polymerase chain reaction (RT-PCR) of Sertoli cells in culture and by biological assay (Senut et al., 1995). RNA is extracted and purified as described (Ausubel et al., 1994). Reverse transcription (RT) is performed using 25-50 ng of total RNA in a reaction mixture containing the RNA, PCR buffer, (10 mM Tris-HCl, pH 8.3, 50 mM KCl), 1 mM dNTP (Boehringer Mannheim, Indianapolis, Ind,), 2.5 nM MgCl₂, 20 U RNAsin (Promega), 100 pM random hexamers (Boehringer Mannheim) and 12.5 U AMV reverse transcriptase (Promega) in a reaction volume of 20 μl. The reaction mixture is incubated in a Perkin-Elmer Thermal Cycler for 75 minutes at 42° C. and 10 minutes at 95° C. RT cDNAs are used immediately for PCR amplification. Two sets of primers are used for PCR amplification: human NT3 (hNT3) (Senut et al., 1995) and porcine β actin (Li et al., 1997). Primer sequences are as follows: hNT3, 5′ primer, (SEQ ID NO: 1), 3′ primer, (SEQ ID NO: 2), 123 bp product, and rat β actin, 5′ primer (SEQ ID NO: 4), 3′ primer (SEQ ID NO: 5). PCR amplification is performed in an 80 μl reaction mixture containing the RT product, PCR buffer, 1.75 mM MgCl₂, 0.5 μg of each primer, 2.5 U Taq polymerase (Perkin-Elmer Cetus, Norwalk, Conn.) and 2 μCi of (³²P)dCTP, in a Perkin-Elmer Cetus Thermocycler as described (Senut et al., 1995). Samples are separated by electrophoresis (PAGE), dried and exposed on x-ray film then developed. The radioactive signal is quantified by measuring the cpm from the membrane by scintillation counting. NT3 values are normalized to the control gene.

NT3 protein production by cells in culture is measured by ELISA (Sadick et al., 1997; Smith et al., 1996) and biological assay as described (Senut et al., 1995). For the bioassay, sympathetic chain and dorsal root ganglia are dissected from embryonic day 9 (ED 9) chick embryos and dissociated by trypsinization. Cells are counted and plated at a concentration of 2000 cells/ well in a 24 well plate coated with 5 μg/ml laminin. Cells are maintained in culture for 48 hours in N2 defined medium (Bottenstein and Sato, 1980) alone or containing recombinant hNT3 (Promega), murine nerve growth factor (mNGF), or supernatant from the virally transfected (NT3) and control Sertoli cells. Cell survival and neurite extension is assessed by viewing under magnification after 48 hours. In this way it is possible to obtain a stable transfectant population of FasL positive Sertoli cells that are producing NT3.

### EXAMPLE 4

Transplantation of genetically modified (rat hNT3-producing Sertoli cells and porcine hNT3-producing RPE cells) into a rat model of spinal cord injury. Description and generation of rat model of spinal cord injury. Multiple spinal and supraspinal pathways influence spinal motor and premotor neurons and local pattern generators to produce locomotion (Grill et al., 1997). Incomplete understanding of the contributions of these elements has complicated the use of animal models for spinal cord injury. However, it is clear that rats with a lesion of the dorsal corticospinal tract (CST) did not sustain long-lasting functional deficits, while those with a more extensive dorsal hemisection did (Grill et al., 1997). For this reason, the present experiment uses an extensive dorsal cord lesion to assess the efficacy of neurotrophin delivery. Dorsal hemisection lesions that interrupted multiple motor projections, including the corticospinal, rubrospinal, cerulospinal, and some raphaespinal, vestibulospinal, and propriospinal tracts are used (Paxinos, 1995).

To create the model lesion, dorsal laminectomies at spinal level T7, are performed on rats (adult Fisher 344 rats 160-200 gm) deeply anesthetized with a mixture (2 ml/kg) of ketamine (25 mg/ml), rompun (1.3 mg/ml), and acepromazine (0.25 mg/ml) (Grill et al., 1997). The dura is opened and bilateral dosal hemisection lesions are performed using a fine-tipped glass-pulled aspiration device (Tuszynski et al., 1996). The dorsal cord midline is identified and superficially incised with microscissors. The lesion is then extended laterally to the edges of the dorsal columns and ventrally to the CST, which lies just dorsal to the central gray matter and the central spinal canal. The CST is aspirated fully at the T7 level and the lesion is extended ventrally and laterally to ensure resection of all CST axons. To achieve complete dorsal hemisection the CST lesion is used as a guide for the desired dorsoventral depth of the lesion and the lesion is extended laterally to remove the lateral aspects of the cord bilaterally. Lesion extent is verified by demonstration of the complete interruption of anterograde transport of horseradish peroxidase conjugated wheat germ agglutinin (HRP-WGA) (see below). Following surgery, animals are kept warm, placed on beds of sawdust, and given manual bladder evacuation for a period of about 10 days and intramuscular ampicillin (25 mg twice daily) to prevent and treat urinary tract infections. Animals regain automatic neurogenic bladder function after 5-10 days (Grill et al., 1997).

Isolation of cells and preparation for transplantation. NT3-producing porcine RPE cells and rat Sertoli cells grown in culture as under Examples 2 and 3, respectively, are harvested from culture plates using trypsin, then rinsed in PBS and collected by centrifugation. Identically grown, non-transfected pRPE and rSertoli cells are used as controls. Cells (2.5×10⁶) are resuspended in a chilled liquid solution (2 ml) of Type I rat tail collagen (Sigma, St louis, Mo.)(Tuszynski et al., 1996). The cell-containing collagen is incubated at 37° C. to promote gelling.

Transplantation of NT3-producing cells (porcine RPE and rat Sertoli cells) into rats with dorsal spinal cord hemisection lesions. Rats are anesthetized with a ketamine mixture as for the lesion surgery (see above). The skin and dorsal dura are opened at the T7 level and the cell-containing collagen pieces are grafted into the hemisection lesion cavities. NT3-producing immune-privileged cells or control cells are implanted as detailed in Table 3. Control subjects received grafts of non-transfected cells or no graft.

TABLE 3

| | Intact | Lesioned | | | Histo- |
|---|---|---|---|---|---|
| | animals/ | animals/ | Functional | PCR | logical |
| | implants | implants | assay | samples | samples |
| pRPE-NT3 | 2 | 6 | 6 | 3 | 3 |
| pRPE control | 2 | 6 | 6 | 3 | 3 |
| rSertoli-NT3 | 2 | 6 | 6 | 3 | 3 |
| rSertoli control | 2 | 6 | 6 | 3 | 3 |

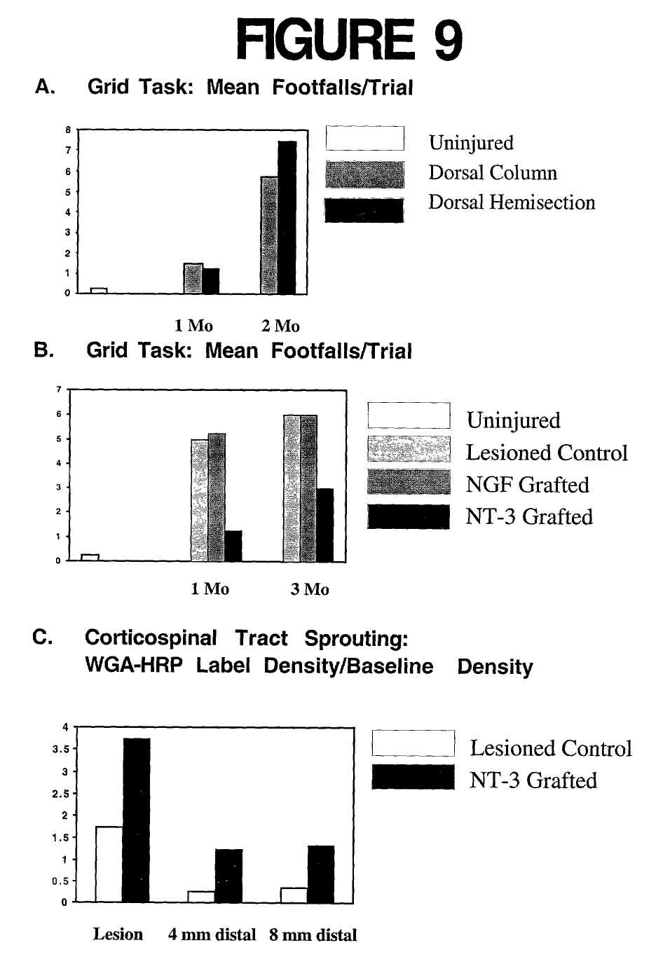

Assay of transplantation effects; alleviation of effects of dorsal spinal cord hemisection and measurement of immune response (see FIG. 9). Lesion completeness is verified by anterograde tracing of the CST tract and Nissl staining at the conclusion of functional testing. To measure anterograde transport in the CST, 12 sites spanning the rostrocaudal extent of the rat sensorimotor cortex are injected with 300 nl of a 4% solution of HRP-WGA (Sigma, St. Louis, Mo.) through pulled-glass micropipettes (40 μm internal diameter) (Paxinos and Watson, 1986) using a PicoSpritzer II (General Valve, Fairfield, N.J.). The pulse frequency and latency are described (Grill et al., 1997). Animals are anesthetized and transcardially perfused two days after HRP-WGA injection using 1% paraformaldehyde/1.25% glutaraldehyde followed by 10% buffered sucrose. Sagital plane sections of the spinal cord are cut (35 μM) and divided into series of six sections. Three of every six sections are reacted with tetramethyl benzidine (TMB) to visualize the HRP-WGA (Mesulam, 1978) and the remaining three sections are Nissl stained. TMB-reacted sections are viewed under dark-field microscopy. Dorsal hemisection lesions show loss of all HRP-WGA transport and loss of dorsal spinal cord white and gray matter (Grill et al., 1997). CST growth in lesioned subjects is determined using HRP-WGA labeling. HRP-WGA granules are quantified using National Institutes of Health (NIH) Image software and measurements are controlled for differences in labeling efficiency between animals by determining a baseline labeling density measurement for each subject (Grill et al., 1997).

Control and NT3-producing cells survived in a similar grafting situation for 6 months (Grill et al., 1997) and there was significant growth of CST axons in the animals that received NT3-secreting grafts compared with controls (Grill et al., 1997). Axon growth was significant up to 8 mm distal to the lesion site but not at 12 mm. Further, CST axons did not penetrate white matter tracts and unlesioned ventral CST. Axon sprouting was not observed which suggests that damaged neurons are responding to NT3 (Grill et al., 1997).

Transgene expression over time is measured in separate animals by performing RT-PCR on freshly dissected NT3 grafts and unlesioned spinal cord. Grafts in 3 animals at each of four time points are tested; 2 weeks, 1 month, 3 months and 6 months. RNA is isolated from fresh tissue (Chomczynski and Sacchi, 1987) and reverse transcribed (1 μg) following manufacturer's instructions (Boehringer Mannheim, Indianapolis, Ind.) using random primers. PCR reactions contained 1/10 of the first-strand synthesis, 0.5 μg of each primer, 1.5 mM MgCl₂, 50 mM KCl, 10 mM Tris-HCl, pH 9.0, 0.1% Triton-X-100, 0.2 mM dNTP, and 2.5 U Taq polymerase (Promega, Madison, Wis.). Amplification was performed for 35 cycles, 1 minute at 94° C., 30 seconds at 60° C and 1 minute at 72° C. using the following primers: 5′ NT3, SEQ ID NO: 1), 3′ nt3, (SEQ ID NO: 2). The housekeeping gene RPL27 is used as a control: 5′ primer (SEQ ID: 6), 3′ primer (SEQ ID: 7), 187 BP product. Aliquots of each reaction were separated by agarose electrophoresis (2%) and stained with ethidium bromide to visualize the 123 bp NT3 product. Unlesioned spinal cord did not show amplification of the NT3 gene, while grafts of NT3-producing cells as old as 6 months still exhibited NT3 production (Grill et al., 1997).

Functional testing of extensive dorsal hemisections of the rat dorsal cord suggest that only the grid locomotion task (GLT) shows sustained functional deficit in animals tested after one month post-lesion (Grill et al., 1997). For this reason, the grid locomotion test was chosen to assess the efficacy of graft-produced NT3. In the GLT, an animal is required to navigate across a 150 cm plastic grid runway to reach a food reward. The runway contains 40×40 mm holes and the test is performed after food deprivation for 48 hours. Animals are tested one and three months after grafting. The subjects are exposed to 5 days of pretraining on the grid, 5 more days of testing with four trials per day. Trials on the last day of testing are quantified using video monitoring. Footfalls below the plane of the grid resulting from failure to grasp a rung are measured. Functional recovery in the context of the GLT is seen in NT3 grafted, but not NGF grafted, animals (Grill et al., 1997).

Measurement of immune response to grafted cells. Grafts that are rejected show dense mononuclear cell infiltration, pronounced expression of CD25 and an upregulation of several cytokines including IL-2, IL-4, IFN-γ (Lehman et al., 1997). Therefore, measurement of the presence of cells producing these cytokines is a method of determining the immune response to the implanted cells. Immune events are followed in the graft by using quantitative reverse transcription-polymerase chain reaction (RT-PCR) and immunohistology. Cytokine gene expression is measured as described (Siegling et al., 1994). Total RNA is prepared from biopsies of each graft and reverse transcribed into cDNA. The cytokine gene expression is quantified using a control fragment that contains primer sequences of rat cytokines and β-actin and HPRT (Lehman et al., 1997). A constant amount of sample cDNA is mixed with varying known amounts of competitor fragment to compete for amplification with specific primers. Proportions of PCR fragments amplified from control fragment and target cDNA are estimated after separation on a 1.5% agarose gel by measuring the intensity of ethidium bromide luminescence with a CCD image sensor. Data is analyzed using the EASY program (Herolab, Weisloch, Germany). cDNA samples are adjusted according to the β-actin and HPRT housekeeping gene signals and the gene expression of T cell markers (CD 3 and CD 25) and cytokines are quantified using the competitive RT-PCR amplification of the target cDNA. Values are expressed in arbitrary units (AU). An AU is the lowest concentration of control fragment that yields a detectable product with one specific primer pair (Lehman et al., 1997). Immune-privileged cells, xenografts and allografts of Sertoli cells, transplanted into rat striatum survived without cyclosporin immunosuppression indicating that the natural production of Fas ligand and other molecules by the Sertoli cells protected the graft in the rat brain from a host immunological response (Saporta et al., 1997). In another study, histological examination of graft bearing-kidneys which had received islet cells co-transplanted with immune privileged cells, negligible lymphocyte infiltration was seen while well-stained insulin positive cells were observed (Korbutt et al., 1997). Thus, the natural production of Fas ligand by cells can confer a very high degree of immune privilege and can enable syngeneic and allogeneic implantation of protein-producing cells with little or possibly no immunosuppression.

### EXAMPLE 5

Creation of transgenic rats with human growth hormone (hGH) expressed in Sertoli cells under control of rat androgen-binding protein (ABP) promoter. Construction of plasmid (ABPp/hGH) transgene in which the human growth hormone (hGH) gene is under control of the rat androgen-binding protein promoter. The rat ABP promoter region, P1, controlling Sertoli cell-specific expression of ABP, (Reventos, 1993) is excised from plasmid J-98 by restriction endonuclease digest with Sstl and HindIII. The ABP P1 DNA fragment containing 619 bp is directly ligated into the 2.1 Kb hGH cDNA-containing plasmid (DeNoto et al., 1981) (Dahler et al., 1994) (see FIG. 6). There are many examples of cell-specific expression of hGH (Dahler et al., 1994) (Archer et al., 1994) including transgenic expression in mouse keratinocytes (Wang et al., 1997) and rat hypothalamic GH-releasing neurons (Flavell et al., 1996). Once constructed, the ABPp/hGH plasmid is used to transform DH5a E. coli cells. Positive clones are picked on the basis of a diagnostic restriction endonuclease cut to determine which clones have the gene inserted in the correct orientation (FIG. 6). Sufficient quantities of the desired plasmid are grown and the DNA is purified by column chromatography (Qiagen, Chatsworth, Calif.). The DNA fragment to be injected is released from the purified plasmid by endonuclease digestion and purified by agarose gel electrophoresis, recovered by Qiaex gel extraction (Qiagen, Chatsworth, Calif.) and redissolved at an appropriate concentration for oocyte injection and aliquoted and frozen for storage. DNA must be purified from plasmid sequences, since these are known to be toxic and will kill embryos, abrogating the possibility of developing a transgenic animal (Hogan, 1986).

Generation of transgenic rats. The ABPp/hGH DNA fragment (2 ng/μl) purified as above, is injected into the male pronucleus of fertilized one-cell rat oocytes using standard techniques (Hogan, 1986). The eggs are cultured overnight and the viable eggs are transferred into the oviducts of pseudopregnant surrogates under halothane anesthesia. Potentially transgenic pups are screened by obtaining tail biopsies under local anesthesia and screening the resultant DNA by Southern blot analysis using a 1 kb Pvull hGH 3′ probe (DeNoto et al., 1981). Alternatively, pup DNAs are screened by PCR analysis (Flavell et al., 1996).

Demonstration of tissue specific expression of hGH and measurement of blood levels of hGH in transgenic animals. Testis and liver from ABPp/hGH transgenic and wild type animals are fixed in 4% paraformaldehyde in 0.1 M sodium phosphate buffer for 24 h prior to embedding in paraffin (Flavell et al., 1996). Tissue sections (4 μ) were incubated with anti-hGH antibody (anti-hGH polyclonal; 1:30,000 dil) overnight then with an avidin-biotin-immunoperoxidase system as previously described (Brown et al., 1993). Control sections are incubated with a non-immune serum or in primary antibody incubated overnight with excess hormone (10 μg/ml hGH). The proportion of hGH positive cells is determined by cell counting using an eyepiece graticle. Double labeling with cytokeratin confirms that expression is restricted to Sertoli cells. Blood levels of hGH are measured by radio immunoassay (RIA) of blood samples (20 μl) from chronically catheterized conscious adult male rats. The assay does not cross-react with rat GH (Fairhall et al., 1992).

Analysis of different tissues by reverse-transcripion/PCR is performed to demonstrate the tissue specificity of hGH expression in the ABPp/hGH transgenic rat. Newborn, 2.5-week and 36-week old transgenic rats and control littermates, identified by Southern analysis, are killed by cervical dislocation and their tissues are frozen in liquid nitrogen and then pulverized. Tissue RNAs are prepared by extraction with TRIzol reagent according to the manufacturer's instructions (GIBCO, Rockville, Md.). RNA samples are treated with RNAse-free DNAse (Promega) and stored at −70° C. prior to use. Reverse transcription is performed on 1 μg of total cellular RNA using a cDNA Cycle Kit (Invitrogen) following the manufacturer's directions. Primers used are: 5′ hGH (SEQ ID: 8); 3′ hGH (SEQ ID: 9); 5′ rat β-actin, (SEQ ID: 4); 3′ rat β-actin, (SEQ ID 5). Reactions are incubated at 94° C. for 5 minutes then 1 unit of Taq DNA polymerase is added. PCR amplification is performed for 35 cycles: denaturation at 94° C. for 1 min, annealing at 59° C. for 1 min, extension at 72° C. for 50 sec, followed by a final extension at 72° C. for 5 minutes. Products are analyzed on a 2% agarose gel (Flavell et al., 1996; Wang et al., 1997).

Cell isolation, purification, tissue culture expansion and cryopreservation of rat Sertoli cells. Rat Sertoli cells from ABPp/hGH transgenic and control rats are isolated and established in culture as described under Example 3. Cells are maintained in culture in Ham's F10 medium containing 10 mM glucose, 2 mM 1-glutamine, 50 μM isobutylmethylxanthine, 100 U/ml penicillan, 100 μg/ml streptomycin, and 5% Lewis rat serum, incubated at 39° C. in 5%CO₂/95% air. Transgene expression is determined by measuring hGH levels in the culture medium by RIA, see above (Fairhall et al., 1992) and by immunocytochemistry of cells plated onto chamber slides (Flavell et al., 1996).

### EXAMPLE 6

Transplantation of genetically modified Sertoli cells from hGH transgenic rat into dwarf rats. Description of dwarf rat model. Growth hormone is the major regulator of mammalian postnatal growth. Spontaneous mutations of the growth hormone gene result in an altered pituitary GH production and cause dwarfism. The dwarf dr/dr rat carries a mutation in the rat GH gene resulting in a truncated, inactive product (Takeuchi et al., 1990). Another spontaneous mutation dw/dw shows a partial GH deficiency (Chariton et al., 1988). Direct replacement therapy alleviates the dwarfism in GH deficient rats (Skottner et al., 1989). Pulsatile replacement is more effective than continuous infusion (Clark et al., 1985; Jansson et al., 1982; Robinson and Clark, 1987), but both of these choices are far more effective than daily injection (Azain et al., 1992; Jansson et al., 1982). Constitutive viral expression of GH also corrects growth deficiency (Hahn et al., 1996) but has the disadvantage of virally infecting the host animal. GH deficiency is associated with small stature, abnormally slow growth of internal organs and abnormally slow bone growth. This example utilizes these physical parameters to assess the effectiveness of implantation of GH-producing cells into dwarf rats.

Cell preparation of transgenic rat hGH-producing Sertoli cells and implantation into dwarf rats. Cultured rat Sertoli cells (see Example 4) derived from high expressing hGH transgenic rats are trypsinized to remove them from the culture plate and pelleted by centrifugation (Korbutt et al., 1997). Cell pellets containing 5-10×10⁶ cells are created in this manner. Control Sertoli cells from non-transgenic littermates serve as negative controls and are prepared in a similar manner (see Example 4).

Mutant rats homozygous for the dwarf mutation (dw/dw), which grow at half the rate of normal animals, are used for these experiments (Charlton et al., 1988). A total of 24, 90-day-old male dwarf rats are studied, in 4 experimental groups. Six animals receive saline and serve as controls, 6 receive implanted hGH-producing Sertoli cell pellets, 6 receive implanted control Sertoli cell pellets and 6 receive recombinant hGH by continuous infusion through an indwelling intravenous cannula (Skottner et al., 1989). The rats are given oxytetracycline (10 mg/kg i.v.) each day for the duration of the experiment. All treatments are maintained for 9 days. To implant cell pellets, rats are anesthetized with halothane and cell pellets collected in polyethylene tubing (PE-50) are gently placed under the left renal subcapsular space (Korbutt et al., 1997).

All the experimental animals are weighed daily. At the end of the 9 day experimental period the animals are sacrificed, final blood samples are collected and the tibia is dissected for assessment of longitudinal bone growth by fluorescence microscopy (Thorngren and Hansson, 1974). A number of other organs are dissected and weighed and the total body weight of each rat is determined. Cell pellets are recovered from beneath the left kidney capsule, fixed in 4% paraformaldehyde and processed for histological determination of the number of remaining cells and the presence of infiltrating immune cells from the host animals (Lehman et al., 1997). Continuous infusion of hGH stimulated body weight gain and bone growth (Skottner et al., 1989). Further, adenovirus-mediated (Hahn et al., 1996) or cellularly delivered (Wang et al., 1997) hGH likewise resulted in growth deficiency correction.

Measurement of immune response to grafted cells. This section describes the measurement of host response to grafted cells, as in Example 5. Grafts that are rejected show dense mononuclear cell infiltration, pronounced expression of CD25 and an upregulation of several cytokines including IL-2, IL-4, IFN-□ (Lehman et al., 1997). Therefore, measurement of the presence of cells producing these cytokines is a method of determining the immune response to the implanted cells. Immune events are followed in the graft by using quantitative reverse transcription-polymerase chain reaction (RT-PCR) and immunohistology. Cytokine gene expression is performed as described (Siegling et al., 1994). Total RNA is prepared from biopsies of each graft and reverse transcribed into cDNA. The cytokine gene expression is quantified using a control fragment which contains primer sequences of rat cytokines and β-actin and HPRT (Lehman et al., 1997). A constant amount of sample cDNA is mixed with varying known amounts of competitor fragment to compete for amplification with specific primers. Proportions of PCR fragments amplified from control fragment and target cDNA are estimated after separation on a 1.5% agarose gel by measuring the intensity of ethidium bromide luminescence with a CCD image sensor. Data is analyzed using the EASY program (Herolab, Weisloch, Germany). cDNA samples are adjusted according to the β-actin and HPRT housekeeping gene signals and the gene expression of T cell markers (CD 3 and CD 25) and cytokines are quantified using the competitive RT-PCR amplification of the target cDNA. Values are expressed in arbitrary units (AU). An AU is the lowest concentration of control fragment that yields a detectable product with one specific primer pair (Lehman et al., 1997).

### EXAMPLE 7

Creation of human RPE cells transfected with tyrosine hydroxylase (TH). Cell isolation, purification, tissue culture expansion and cryopreservation of human RPE cells. Eye banks are sources of primary human immune-privileged cells and tissues that naturally express Fas ligand, including the ciliary body, corneal epithelium, and corneal endothelium, retina and retinal pigment epithelium. Human eyes from deceased donors are removed by a surgeon and placed in a sterile solution, ideally within six hours of death, and then transported to the nearest eye bank where each eye is placed in an antibacterial bath, and examined for physical defects. Blood tests are conducted to determine that an eye is free from disease-causing organisms such as human immunodeficiency virus (HIV) and hepatitis B. Surgeons remove tissue desired for transplantation, usually the cornea, which is used in more than 46,000 corneal transplant surgeries annually in the U.S. to correct vision loss resulting from birth defects or from swelling or scarring of the cornea caused by infection, burns, or a blow to the head. Sclera (the white tissue) is used in cataract and reconstructive surgery, and vitreous humor (the clear gel that fills the back of the eyeball and holds the retina in place) is used to repair detached retinas (Microsoft, 1998). Human RPE cultures are established from explants from human eyes obtained from donors (Esser et al., 1997; Liu et al., 1997; Lu et al., 1995). Eyes are rinsed in phosphate buffered saline (PBS) containing antibiotics (100 U/ml penicillan and 100 U/ml streptomycin). The anterior segment, retina and vitreous humor are removed. Eye cups are incubated at 37° C. in 5% CO₂ with 0.3% trypsin in Ca⁺⁺/Mg⁺⁺ free PBS, containing 0.5 mM ethylene diamine tetraacetic acid (EDTA) for 45 minutes (Esser et al., 1997; Jaffe et al., 1990). The retinal pigment epithelium is dislodged from Bruch's membrane and cells are gently triturated to achieve a single cell suspension which is plated in Dulbecco's modified Eagle's medium, supplemented with 10% fetal calf serum, 100 U/ml penicillan and 100 U/ml streptomycin (Kutty et al., 1994). Retinal pigment epithelial origin and maintenance of phenotype is confirmed by cytokeratin immunocytochemical analysis (Esser et al., 1997). This technique yields pure RPE cultures, free of contaminating choroidal cells (Jaffe et al., 1990). Media are changed twice weekly and cells are grown to confluency on 75-cm flasks. Subsequent passaging of cells is performed by trypsinization using standard protocols.

Vector construction of retrovirus containing human tyrosine hydroxylaselaromatic amino acid decarboxylase (TH/AADC) expression cassette under control of RPE-specific promoter (RPE65). The viral introduction of tyrosine hydroxylase (TH) into animal models of Parkinson's disease (PD) has shown that this rate-limiting enzyme in catecholamine synthesis ameliorates PD symptoms presumably by effecting the production of L-dopa (During et al., 1994; Horellou et al., 1994; Kaplitt et al., 1994). Since TH only produces L-dopa, it is presumed that endogenous aromatic amino acid decarboxylase converts L-dopa to the active dopamine (DA) in the tissue. The efficiency of such conversion is unclear however and the coexpression of TH and AADC in an expression cassette results in a greater production of DA (Moffat et al., 1994). In the current study this expression cassette is used, under the control of an RPE-specific promoter, to effect the expression of DA in immune-privileged cells from human donor eyes.

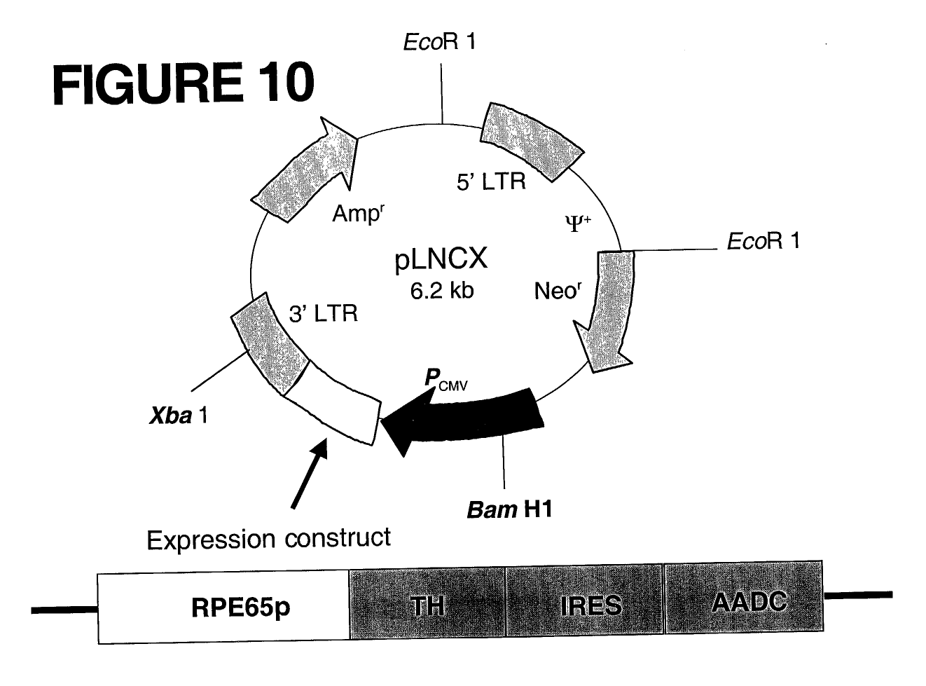

A bisistronic expression cassette is created as described (Moffat et al., 1994). In this plasmid, the TH and MDC cDNAs flank the encephalomyocarditis internal ribosome entry site (IRES) sequence which allows the production of both proteins (Ghattas et al., 1991). The TH/AADC are cloned into a backbone containing the RPE65 promoter, which is a tissue specific promoter active only in RPE cells (Nicoletti et al., 1998) and the SV40 poly(A) signal (FIG. 10). This plasmid construct is used to transform DH5α E coli cells. Individual transformants are grown, harvested and analyzed by restriction endonuclease mapping to identify bacterial clones with the desired plasmid in the correct orientation. The RPE/TH/AADC, which has the TH/AADC cDNA under the control of the RPE65 promoter (see FIG. 10) is then cloned into retroviral expression vector pLNCX (Clontech, South San Francisco, Calif.), which carries the extended viral packaging system and the neomycin resistance gene (FIG. 4). The resultant RPEpLNCX vector is used to transform DH5α E. coli cells that are then restriction, endonuclease-mapped to identify clones with the plasmid in the correct orientation. Packaging cells (Clontech) are transfected by the calcium phosphate coprecipitation method (see Example 1). Each plate of cells is transfected with 10-15 μg of plasmid DNA. Stable virus producing cell lines are selected by maintaining the cells in selection medium, containing G418 (0.5 mg/ml) for 1 week following transfection. Viral titer is determined and individual high titer clones are selected following screening of 20-50 clones. High titer clones are then expanded, and maintained as frozen stocks. Cells are grown in the absence of G418 for viral production. Supernatant culture medium from confluent cultures of high viral titer cells is collected, filtered to remove remaining cells (0.45 μ filter, cellulose acetate or polysulfonic low protein binding) and stored at −80° C. or used immediately. Aliquots are frozen depending on viral titer since repeated freezing and thawing reduces the titer.

Retroviral infection of RPE cells, selection, tissue culture expansion and cryopreservation. To achieve viral infection, hRPE cells are plated 12-18 hours prior to infection at a cell density of 3-5×10⁵ cells per 100 mm plate in complete culture medium containing heat-inactivated serum, which lacks complement that could inactivate retrovirus (Mochii et al., 1998). Filtered virus-containing medium obtained from the packaging cells (see above) is placed on the cells and polybrene is added to the culture to a final concentration of 4 μg/ml. Complete culture medium is replaced after 24 hours. In general, half-maximal infection takes place after 5-6 hours and maximal infection takes place after 24 hours. Viral reverse transcription and integration takes place between 24 and 36 hours following infection. Expression of the transgene can be observed as early as 24 hours post infection and usually reaches a maximum at about 48 hours. At this point cells are subjected to selection with G418 (1 mg/ml). The cells are also examined by PCR to confirm that no wild-type virus is present in the culture supernatant. Selected cells are grown to 80% confluence in the presence of 0.5 mg/ml G418 then passaged by trypsinization and aliquots are frozen by standard techniques (Tezel et al., 1997).

In vitro characterization of hTH-producing immune-privileged hRPE cells. Human RPE cells infected with the RPEpLNCX virus are characterized to determine dopa and DA release. Two different paradigms are used to measure release from cells in culture (Lundberg et al., 1996). In the first, cells are maintained in culture in complete Dulbecco's modified Eagle's medium as in example 2 and 1 ml aliquots are removed on days 4, 6, 8, and 10 post-transfection. In the second experiment, dopa and DA release is studied for 3 to 4 weeks. To do this, individual cultures are created at the outset on 4-well plates (Nunc/Nalgene, Rochester, N.Y.). On the day of the determination the cells are washed with Hank's balanced salt solution (HBSS) and 250 μl of Kreb's ringer solution is added to the culture and changed every thirty minutes. In this way it is possible to determine the DA production as a function of time in culture. Dopa and DA are determined by radioenzymatic assay as previously described (Schmidt et al., 1982). Analysis is performed on four independent cultures at each time point/condition investigated. Sample size is 25 μl for dopa content and 20 μl for DA content and the detection limit of this technique is approximately 30 fmol for DA and 20 fmol for dopa (Lundberg et al., 1996). Transfection of the (TH/AADC) expression cassette into COS-7 cells resulted in 5.1 nmol/mg/min TH activity and 13.98 nmol/mg/h AADC activity, and roughly five-fold higher production of DA than in cells transfected with TH alone (Moffat et al., 1994).

TH immunocytochemistry is performed on cells grown on polyornithine-coated cover slips. Cells are plated onto previously coated coverslips the day prior to staining. Cells are fixed in 4% paraformaldehyde in phosphate buffered saline (PBS) for 10 minutes on ice. Following fixation, the cells are rinsed gently three times with cold PBS then blocked with 10% normal goat serum (NGS) in PBS for 1 h at room temperature. Samples are incubated with anti-TH antibody (PelFreez, 1:500) in 10% NGS in PBS overnight at 4° C. The following day the samples were rinsed three times with cold PBS then incubated with rhodamine-conjugated goat anti-rabbit (Dakopatts, UK, dil 1:50) for one hour at room temperature then rinsed to remove unbound antibody and mounted on glass slides in Vectashield (Vector Labs, Burlingame, Calif.).

Dopa production by cells in culture was maintained as long as four weeks in retrovirally transformed cells with the TH gene under the control of viral promoters (Lundberg et al., 1996). Immunohistochemistry demonstrated that 80-100% of the cells were producing dopa and that cells that became more mature over time produced more dopa than in early cultures (Lundberg et al., 1996). The maintenance of FasL production by human RPE cells in culture is determined as under examples 2 and 3.

### EXAMPLE 8

Transplantation of genetically modified, hTH-producing, immune-privileged human RPE cells into a rat model of Parkinson's disease. Parkinson's disease (PD) is a degenerative neurological disorder characterized by a progressive loss of dopaminergic cells and a deficiency of tyrosine hydroxylase (TH), which is required for the synthesis of dopamine (DA) (Agrid et al., 1987). Early in the disease, TH deficiency can be ameliorated somewhat by oral administration of L-dopa (Barbeau, 1961; Birkmayer and Hornykiewicz, 1961), which is converted in the cell by aromatic amino acid decarboxylase (ADDC) to DA. A rat model of PD developed by Jun (Jun et al., 1994), which involves a partial lesion created with 6-hydroxydopamine (6-OHDA), is used to determine the efficacy of administration of TH/ADDC by transduced cell implantation. This model has a substantial reduction in striatal dopaminergic innervation, resulting in ipsiversive rotation in response to amphetamine. This lesion therefore provides an opportunity to study sprouting from the remaining dopaminergic fibers and better mimics the neuropathology of human Parkinson's disease (Yoshimoto et al., 1995).

To create partially lesioned rats a modification (Yoshimoto et al., 1995) of the method of Jun is used (Jun et al., 1994). Rats are deeply anesthetized with a mixture of chloral hydrate (17 mg/kg)/sodium pentobarbital (3.5 mg/kg) administered intraperitoneally. The rats are placed into a stereotaxic apparatus with the bite bar set at zero. The coordinates for a unilateral injection are: AP, −5.5 mm; ML, 2.0 mm, DV, 7.1 mm with respect to bregma and the skull (Yoshimoto et al., 1995). An injection rate of 1 μl/min using a Hamilton syringe is used and the needle is left in place for 2 min and then withdrawn at 1 mm/min. Counts of tyrosine hydroxylase positive neurons in the substantia nigra pars compacta (SNpc) and the ventral tegmental area (VTA) show the extent of the 6-OHDA lesion. The percent lesion is calculated as the number of TH-immunoreactive neurons on the lesioned side relative to the number on the unlesioned side. In general the percent lesion in these two areas is around 85% (Yoshimoto et al., 1995). Further, each animal is tested for rotational response to apomorphine or amphetamine.

Implantation of genetically modified hRPE cells. Confluent 10 cm plates of cultured RPE/LNCX cells or normal controls are removed from the plate with 0.05% trypsin in PBS and transferred into PBS containing glucose (1 mg/ml) and serum, to inactivate the trypsin. The cells are washed twice and resuspended in PBS at a density of 20,000 cells/μl and injected stereotaxically into four sites in the right striatum at the following coordinates: AP 0.7 mm, ML 3.0 mm, DV 4.5 mm and 5.5 mm; and AP 0.0 mm, ML 3.0 mm, DV 5.0 mm and 6.0 mm with respect to the bregma and the skull (Yoshimoto et al., 1995). A total of 200,000 cells are injected, divided between the four sites. Lesioned animals serving as controls receive identical injections of unmodified hRPE cells.

Rotational behavior assay. In rats with unilateral 6-OHDA lesions, manipulation of the DA system can be monitored in a quantitative manner by measuring rotational behavior. One week following the 6-OHDA lesion the rats are tested for rotational behavior. This is done by injecting rats with amphetamine sulfate (5 mg/kg, 5 mg/ml in sterile saline, i.p., Sigma, St Louis, Mo.) then testing for 90 minutes after first allowing the drug to take effect (10 minutes). The second week, postlesion animals are tested for apomorphine induced rotation. Testing begins 2-5 minutes after injecting apomorphine (0.25 mg/kg, 0.25 mg/ml, in sterile saline, s.c., Sigma, St Louis, Mo.) and is continued for a duration of 1 hour. Rats are videotaped and the number of rotations was counted blindly. Rats rotated at least 300 turns over 90 minutes in response to amphetamine and less than 250 turns over 60 minutes in response to apomorphine are selected as partially lesioned (Yoshimoto et al., 1995). These rats receive implants of human RPE cells that are expressing TH/AADC or untransfected hPRE cells. Experimental and control subjects are retested for amphetamine-induced (14 and 32 days postimplantation) and apomorphine-induced (18 and 36 days postimplantation) rotation. Brain derived neurotrophic factor (BDNF)-producing astrocytes implanted similarly into lesioned animals resulted in the attenuation of amphetamine-induced rotation by 45% at 32 days after grafting and immunohistochemical analysis of the tissue post mortem suggests that the transgene is expressed for up to 42 days (Yoshimoto et al., 1995). Implantation of DOPA-producing astrocytes into a partially lesioned rat resulted in the 50% attenuation of apomorphine-induced rotation 2 weeks postimplantation (Lundberg et al., 1996). Microscopic examination revealed that only a few percent of transplanted cells maintained transgene expression, which was under the control of the CMV promoter (Lundberg et al., 1996). The use of tissue-specific promoters, such as the RPE65 promoter, results in continuous transgene expression in vivo, while many of the viral promoters have been shown to down-regulate in vivo (Gage et al., 1997).

Immunohistochemical analysis of implanted cells and experimental animal brains. Immunohistochemical analyses are performed to examine TH expression in brains that receive cell implants. Two weeks posttransplantation rats are deeply anesthetized with chloral hydrate (400 mg/kg, i.p.) and transcaridally perfused with ice-cold 4% paraformaldehyde in 0.1 M phosphate buffer, pH 7.4. Brains are postfixed 6-8 hours in the same fixative then soaked overnight in 0.1 M phosphate buffer containing 20% sucrose. Brains are cut on a freezing microtome throughout the striatum and sections are collected for further processing. Tissue sections are treated with 3% H₂O₂ in potassium phosphate buffer pH 7.2 prior to processing to quench endogenous peroxidase activity. Sections are first incubated in 5% normal swine serum plus 0.3% Triton-X-100 to block unspecific antibody binding. Sections are then incubated with a rabbit antiserum against TH (Pel-Freez, Rogers, AR 1:500) overnight at 4° C. The following morning the sections are rinsed with PBS then incubated with biotinylated swine anti-rabbit immunoglobulins (Dakopatts, UK, dil 1:200) for 1 hour at room temperature. Following this incubation, sections are again rinsed in PBS then incubated with an avidin-biotin-peroxidase complex (Vectastain, Vector Labs, Burlingame, Calif.) using 3,3-diaminobenzidine (DAB, Sigma, St. Louis, Mo.) as chromogen (0.05% DAB and 0.03% H₂O₂ in PBS, 1-2 min). Sections are then mounted onto chromealum-coated slides and coverslipped with DPX (British Drug House, UK). In animals transplanted with TH-producing astrocytes, large numbers of astrocytes were observed in the brain tissue (Lundberg et al., 1996). Most of the TH-expressing cells were seen in the central graft, but some of them had migrated away from the graft site, as much as 600 μm (Lundberg et al., 1996). Implantation of TH-producing fibroblasts resulted in a significant decrease in the rotational behavioral of lesioned rats but did not demonstrate TH immunoreactivity (Gage et al., 1997).

Measurement of immune response to grafted cells. The measurement of host response to grafted cells is preformed as described under Example 5. Grafts that are rejected show dense mononuclear cell infiltration, pronounced expression of CD25 and an upregulation of several cytokines including IL-2, IL-4, IFN-γ (Lehman et al., 1997). Therefore, measurement of the presence of cells producing these cytokines is a method of determining the immune response to the implanted cells. Immune events are followed in the graft by using quantitative reverse transcription-polymerase chain reaction (RT-PCR) and immunohistology. Cytokine gene expression is performed as described (Siegling et al., 1994). Total RNA is prepared from biopsies of each graft and reverse transcribed into cDNA. The cytokine gene expression is quantified using a control fragment which contains primer sequences of rat cytokines and 13-actin and HPRT (Lehman et al., 1997). A constant amount of sample cDNA is mixed with varying known amounts of competitor fragment to compete for amplification with specific primers. Proportions of PCR fragments amplified from control fragment and target cDNA are estimated after separation on a 1.5% agarose gel by measuring the intensity of ethidium bromide luminescence with a CCD image sensor. Data is analyzed using the EASY program (Herolab, Weisloch, Germany). cDNA samples are adjusted according to the β-actin and HPRT housekeeping gene signals and the gene expression of T cell markers (CD 3 and CD 25) and cytokines are quantified using the competitive RT-PCR amplification of the target cDNA. Values are expressed in arbitrary units (AU). An AU is the lowest concentration of control fragment that yields a detectable product with one specific primer pair.

### EXAMPLE 9

Delivery of Neurotrophin-3 by Allogeneic Sertoli Cells in Rat Model of Spinal Cord Injury. Neurotrophic factors are key nervous system regulatory proteins that modulate neuronal survival, axonal growth, synaptic plasticity and neurotransmission. They have been shown to elicit renewed axonal growth and remyelination following spinal cord injury (Bradbury et al., 1999; Bregman et al., 1997; Grill et al., 1997; Kobayashi et al., 1997; McTigue et al., 1998), and are very likely to be an important component of successful therapeutic regimens for spinal cord injury. Large proteins such as neurotrophins are unable to cross the blood-brain barrier (9) and therefore if given systemically will not be able to reach the corticospinal neurons that are often affected by spinal cord injury.

Generally neurons in the CNS of adult mammals cannot regrow or adequately remyelinate the axons that have been damaged. This is not due to intrinsic properties of the neurons but rather results from their environment. Neurons elsewhere in the body and during development in the spinal cord and brain can regenerate (reviewed in (Bregman et al., 1998)). A number of experiments in animals have shown that axons in the CNS can be stimulated to regrow significant distances when provided a particular environment. Neurotrophins appear to be critical in creating an environment that supports regeneration (Bregman et al., 1998; Jones et al., 2001). The presence of inhibitory molecules in the spinal cord and the lack of molecules that stimulate neuronal survival and growth combine to effectively stop axonal regeneration in the CNS (Bregman et al., 1998; Jones et al., 2001; Steeves and Tetzlaff, 1998; Stichel and Muller, 1998). The sequence of events that occur in acute spinal cord injury is complex and would be very difficult to completely reverse or prevent. Importantly, however, it is thought that preserving as few as 10% of the normal number of axons can have a profound effect on locomotor recovery (McDonald, 1999).

Genetically modified rat primary Sertoli cells expressing and secreting human NT-3 are prepared and characterized. We cloned hNT-3 into the Ad5 expression vector and then obtained viruses from University of Iowa Gene Transfer Vector Core facility that either expresses green fluorescent protein (GFP) or GFP and NT-3. Syngeneic and allogeneic Sertoli cells modified to express GFP or GFP and NT-3 are implanted in the spinal cord of uninjured and injured animals. We will show here the survival of the cells in both the spinal cords for as long as fifteen days. Most importantly, injection of the cells into the spinal cord did not elicit an immune response, specifically did not activate the production of macrophages. Implantation of the cells in injured spinal cord did not additionally increase the number of activated macrophages produced. NT-3 expression is observed in the spinal cord at 15 days post implantation of the cells. Our in vitro quantitative analyses of the amount of NT-3 secreted indicated that the cells produce 20-50 ng NT-3/10⁵ cells/24 h. Secretion of biologically active hNT-3 is confirmed by specific elicitation of neurite outgrowth from cultured murine embryonic neuronal cells due to the hNT-3 in the cell culture supernatant of the genetically modified Sertoli cells.

Isolation of Sertoli Cells. Sertoli cells are isolated from the testes of 15-21 day-old male rat pups (either Sprague Dawley or Lewis strains) following the protocol described by Korbutt et al., (Korbutt et al., 1997). Briefly, wet the rat body with ethanol and open the abdominal cavity. Remove the tunica from each testes and place in a tube containing 1× HBSS. Remove excess solution and the outer connective tissue. Weigh the tissue and chop into finer pieces. Transfer to 50-ml tube containing 45 ml of 1× Hank's Balanced Salt Solution (HBSS), 5 ml of trypsin solution (1.25 mg/ml) and 100 □l of DNase (6.64 mg/ml). Incubate at 37° C. for 25 min with occasional but gentle swirling. Allow the tissue to settle and aspirate the supernatant. Add 5 ml of trypsin inhibitor and allow settling for 1-2 min. Wash with Hanks (3×). After the final wash add 5 ml of collagenase (0.7 mg/ml), 5 ml of HBSS and 50 □l of DNase (6.64 mg/ml). Than add 10 ml of HBSS and incubate at 37° C. for 15 min with occasional swirling. Spin at 2,000 RPM for 2 min. Wash in the same way twice again. Resuspend pellet in F-12 Ham's medium (without serum) containing 100U/ml penicillin, 100 □g/ml streptomycin and plate on tissue culture plates. Incubate at 37° C. in a 5% CO₂ incubator. Fetal bovine serum (FBS) is added to the medium at 10% concentration and cells are propagated in that medium. We have successfully isolated, propagated, and grown the cells from frozen stocks.

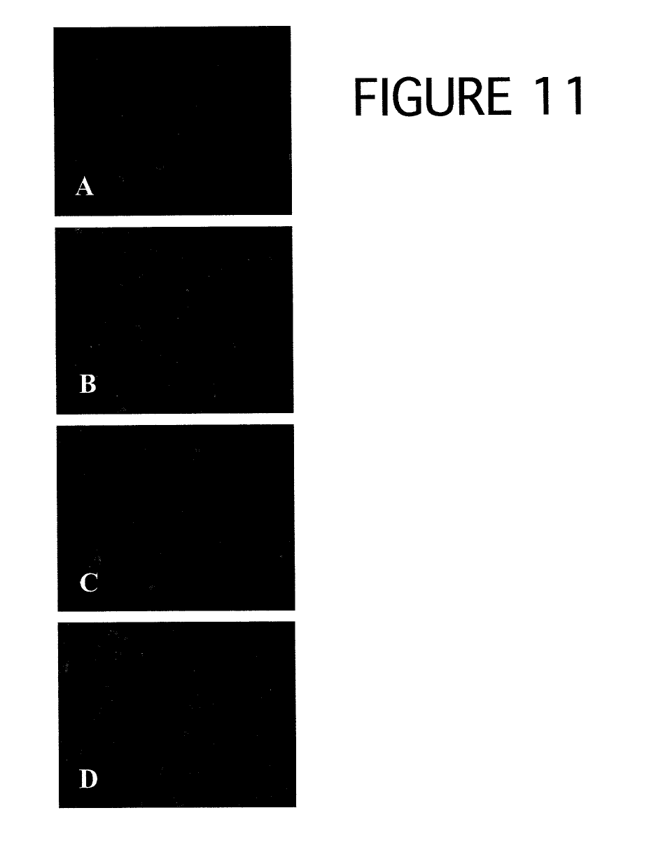

Staining Sertoli cells with antibody to follicle-stimulating hormone receptor (FSHr). Lewis Sertoli cells (1-2×10⁴) are plated into each well of an 8-well Nalge Nunc Lab-Tek Chamber Slide, and then covered and grown in a CO₂ incubator at 37° C. for 2 days. The cells are fixed with 50:50 acetone:ethanol solution at 4° C. for 10 min and the solution aspirated and the cells allowed to air day. The sample is stored in a 50:50 mix of PBS:glycerol at 4° C. The PBS:glycerol solution is removed and the samples washed twice with PBS at RT for 5 min just prior to staining. To block nonspecific binding of secondary antibodies the slide is incubated in PBS with 10% normal rabbit serum, 0.2% Triton X-100, and 0.1% bovine serum albumin (BSA) at RT for 3 h. This solution is removed and the slide blocked by PBS with 2% normal rabbit serum, 0.2% Triton X-100, and 0.1% BSA (2% rabbit-TX-BSA) at RT for 10 min. The primary antibody (Ab; sheep Ab to FSHr; Biogenesis, Brentwood, N.H.) (Korbutt et al., 2000) is diluted with 2% rabbit-TX-BSA from stock (7.2 mg/ml) to give a final concentration of 2 □g/ml, 4 □g/ml or 8 □g/ml in each well. After adding the diluted Ab the plate is incubated at 4° C. overnight. The primary Ab is removed, and the plate is washed three times with PBS at RT for 5 min. The secondary Ab (biotinylated rabbit anti-goat IgG) is diluted 1:150 in PBS and incubated with the slide at RT for 60 min. The slide is washed three times with PBS at RT for 5 min. Then the slide is incubated with avidin conjugated Alexa Fluor 350 (1:1 000 dilution in PBS; Molecular Probes, Oreg.) at RT for 1 h. This is followed by three PBS washes, and then the slide is allowed to air day and after adding mounting medium (Vectashield, Vector Labs, Burlingame, Calif.) a coverslip is placed on it. Fluorescence is evaluated at 200× magnification, using a Nikon Optiphot microscope equipped with an epi-fluorescence attachment (FIG. 11) in conjunction with SPOT 1 digital camera (Diagnostic Instruments, Sterling Heights, Mich.) and Photoshop 6.0 (Adobe, San Jose, Calif.).

Injections of cells into spinal cord. Before implantation, the cells are rinsed twice with PBS, trypsinized and resuspended in serum free medium at a concentration of 1×10⁵ cells/□l. The cells are implanted close to the site of injury with a 32-guage beveled needle with a 45-degree angle 2 inches in length (Hamilton # 0160832). The needle is attached to a 10-□l Hamilton syringe and a Harvard apparatus. Before implanting, an incision are made with 30-guage needle and the new needle placed into the incision position. Cells are implanted at a rate of 0.2 □l/min. A total of 2 □l are implanted and needle left in for an additional 5 min. Animals are sutured and kept in the recovery chamber till they gain consciousness.

Experimental model. A standardized model of contusion spinal cord injury in rats as developed by investigators at the Multicenter Animal Spinal Cord Injury Study (MASCIS) is used. Adult male Sprague-Dawley rats (N=24), weighing 300-350 g is used in this study. Animals are anesthetized with 4% chloral hydrate (0.9 ml/100 g body weight) intraperitoneally and prepared for spinal cord injury. Rectal temperatures are maintained at 37° C. with a heating pad. A T8 laminectomy is performed. The spinal cord of each rat is subjected to a contusive injury as described previously (Noble and Wrathall, 1989; Wrathall et al., 1985). Briefly, after the laminectomy each rat is slightly suspended by clamps attached to the spinous processes above and below the laminectomy site to minimize the effect of the respiratory cycle at the time of impact. This model uses an impact device designed by Gruner and Young at New York University (Constantini and Young, 1994; Gruner, 1992) device rod (10 g) is dropped at a distance of 5 cm onto the exposed cord. This results in a moderate contusive injury, as defined by anatomical and behavioral parameters (Noble and Wrathall, 1985; Wrathall et al., 1985). After the production of injury, modified Sertoli cells are implanted into the spinal cord, as described above. The fascia and muscle layers are subsequently sutured, and the skin closed. Rats recover from surgery in temperature and humidity controlled incubation chambers. They are transferred to their home cage, and bladder evacuation is accomplished using the method of Crede until bladder function returns that is usually within 2 to 3 weeks.

Tissue processing for analysis. Animals are re-anesthetized and euthanized at 42 days after injury. Animals are perfused with 4% paraformaldehyde in 0.1 M PBS. Spinal cord of about 2 cm is isolated and is divided into a minimum of three segments, corresponding to the site of contusion and 1 cm proximal and distal to the contusion. The tissue is post-fixed for 4 h, cryoprotected in 20% sucrose for 3 to 4 days and stored at −70° C. in OCT medium. 14-□m thick transverse sections are made on a cryostat and collected onto Fisher SuperFrost Plus slides. Sections are stored at −70° C. and are analyzed as described below. Every 5^{th} section is used for each of the analysis and the rest of the sections are stored as a library for any further staining needed.

Lesion volume. Luxol Fast blue staining of the tissue is done to look for degeneration of the myelin sheath. Every 5^{th} section of the spinal cord from all the experimental animals is stained with Luxol Fast blue as follows. Rehydrate sections in PBS followed by incubation in water. Dehydrate in 70% EtOH for 15 min followed by staining with Luxol Fast Blue at 50° C. for 18 h. Cool to RT for 30 min followed by wash with 95% EtOH and water. Destain with 0.05% Li₂CO₃ for 2 min, 70% EtOH for 2 min and water for 2 min. Dehydrate with 95% EtOH for 2 min (2×) and 100% EtOH for 2 min (2×). Coverslip and observe slide under dark field. The picture is analyzed for area measurements using the Photoshop 6.0 software (Adobe, San Jose, Calif.). After all the analyses are completed, final statistical analyses are done as described below.

Amount of inflammation. Immune reaction in the spinal cord in response to the injury and the injected cells is analyzed qualitatively to determine the presence of macrophages and microglia. Sections that are proximal and distal to the site of injury and implantation are stained. The protocol followed is the same as described in section B.6.9.

Axonal growth in spinal cord. Sections obtained from all the spinal cords are analyzed for axonal growth by staining with antibodies specific for the various axonal processes. Selected sections, several proximal to the injury site and several that are distant, are analyzed. Previous studies have provided evidence to suggest that both sensory and motor fibers are likely to respond to NT-3 (DiStefano et al., 1992; Henderson et al., 1993; Hohn et al., 1990). The in vivo analysis of axonal growth in response to NT-3 confirms in vitro observations. For this analysis the sections are stained with NGF receptor (p75), calcitonin gene-related peptide (CGRP) (Serotec, Killington, OX, England) and tau I antibody (Roche, Indianapolis, Ind.) that is specific for axonal processes will also be used. This staining reveals if the sensory and motor neurons are affected by NT-3. Specific sprouting of host sensory neurites in response to hNT-3 producing grafts is expected in the rat spinal cord. A significant density of the axonal processes invading the grafts is immunoreactive for the low-affinity receptor for NGF and for CGRP (Senut et al., 1995). Control experiments are carried out by replacing the primary Ab with normal serum. Under these conditions, the immunohistochemical labeling is completely abolished. The primary Abs are diluted, according to the vendor's protocol in their respective blocking sera and incubated with the sections overnight. The secondary Ab is biotinylated and final color development uses avidin conjugated alexa fluor dye.

Cloning of NT-3 into the Ad5 expression vector. Human NT-3 gene is subcloned into the adenovirus expression vector pAd5CMVk-NpA. NT-3 gene is excised from the plasmid pBSNT-3 with EcoRI and BamHI. This gene fragment is further gel purified and ligated with pAd5CMVk-NpA, a shuttle plasmid containing the inverted terminal repeat (ITR) of the adenoviral genome, encapsidation sequences and adenoviral sequences necessary for subsequent homologous recombination.

Transcription of NT-3 is controlled by the cytomegalovirus promoter (CMV) and terminated at the polyA sequences. The replication-deficient adenoviral vector is deleted in regions E1 and E3. Once the clone is a confirmed positive, plasmid DNA is isolated and purified using the Qiagen Maxiprep kit (Qiagen, Calif.). The University of Iowa Gene Transfer Vector Core facility makes replication incompetent adenovirus that expresses either GFP alone or both NT-3 and eGFP (Ad5-CMV-NT-3/eGFP) (Anderson et al., 2000). Adenoviral stocks are produced and titered on 293 cells according to standard methods. Viral titers obtained ranged from 1×10¹¹ to 1×10¹² particles/ml.

Viral infection. To enable long term NT-3 expression by the cells, they are infected with a replication-deficient adenovirus that expresses eGFP that is obtained from the Gene Transfer Vector Core facility, University of Iowa, Iowa City, Iowa. The protocol is as follows: 20,000 cells (Sertoli) are plated per well of the 8 well chamber slide a day before the infection. The stock viral concentration is 1×10¹² particles/ml or 1×10⁹ particles/□l. Perform serial dilutions to give 1×10⁸ particles/□l, 1×10⁷ particles/□l and 1×10⁶ particles/□l. Cells are infected at 100 particles/cell, 1,000 particles/cell, 10,000 particles/cell and 100,000 particles/cell. Control cells are not infected with any virus. Before infection the cells are washed three times with serum free medium (SFM) and incubated with the respective viral concentration at 37° C. for 4 hours. At the end of the infection period add serum-containing medium and continue incubation at 37° C. Cells are analyzed 3 and 6 days post infection by fluorescence microscopy. The number of cells infected increased with the number of virus particles used per cell. At 10,000 particles per cell most of the cells are infected, but at the higher concentration there is cell death. Hence, following this experiment the cells are infected at 10⁴ viral particles/cell.

Measurement of NT-3 expression. The amount of NT-3 secreted by the Sertoli cells infected with Ad5-CMV-NT-3/eGFP is determined by ELISA (Promega, Madison Wis.; NT-3 Eₘₐₓ Immunoassay kit). For the implantation experiments and in vivo NT-3 bioactivity assays, cells are infected at 10⁴ particles per cell. Twenty-four hours post infection the supernatant is collected and assayed for NT-3 following the directions supplied by the vendor (Promega). Briefly, a sandwich ELISA is performed using polyclonal antibody to NT-3 for coating the plates and a mAb for detection. A Thermomax microplate reader (Molecular Devices, Sunnyvale, Calif.) is used to record the absorbance at 450 nm. A microcomputer based software program called SOFTmax developed by Molecular Devices controls the microplate reader and does data handling and analysis.

Viral particles are titrated on a per cell basis, and the supernatant obtained from each well is analyzed by ELISA. With increasing viral concentration there is an increase in the amount of NT-3 secreted. Cells infected with a concentration of 10⁴ particles per cell, produced within 24 hours a supemate containing 37 ng/ml based upon standard NT-3 curve. After repeating the infection and ELISA three times the per cell concentration is determined to be between 0.1 to 0.3 pg. Hence with the number of cells that are being implanted (2×10⁵ cells), there is 20-50 ng of NT-3 produced in 24 hours. The amount of NT-3 produced is similar to that used by researchers to study the effect of trophic factors in vitro and in vivo. Astrocytes infected with Ad/NT-3 produced NT-3 at a range of 2-5.5 ng/ml. This concentration is also within the bioactivity range concentration reported as tested on chick ciliary ganglion neurons (Smith et al., 1996). NT-3 has also been shown to function as a mitogen on neural crest cells in culture. Again the effective concentration range is from 0.1 to 10 ng/ml (Kalcheim et al., 1992). Recombinant mouse NT-3 produced from a vaccinia virus at 200 ng/ml was shown to be biologically active. The concentration of recombinant NT-3 that allows half-maximal survival of sensory neurons is determined to be 25 pg/ml (Gotz et al., 1992).

Implantation and survival of syngeneic cells into the rat spinal cord. Laminectomy is performed on male Sprague Dawley rats to expose the T8 disc of the spinal cord. Sertoli cells (isolated from Sprague Dawley pups) are infected with the virus Ad5GFP, 24 hours before implantation. Cells are implanted with a 32-gauge needle hooked to a Hamilton syringe and a Harvard apparatus. Before implanting, an incision is made with 30gauge needle and the new needle is placed into the incision position. Cells that are harvested are resuspended at a concentration of 1×10⁵ cells/□l and are implanted at a rate of 0.2 □l/min. A total of 2 □l is implanted and needle is left in for an additional 5 min.

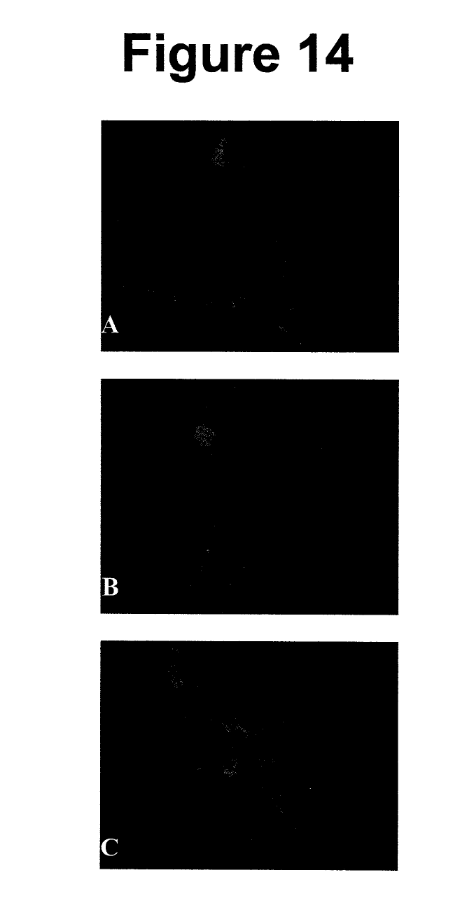

We observe green fluorescence from the GFP in the sections that are obtained three days after implantations. Most intense fluorescence is observed in the sections that are closest to the site of implantation and tapered down moving away from the injection site in both the directions (FIG. 14A).

Implantation and survival of allogenic cells in the rat spinal cord. All the procedures for the cell implantation are the same as mentioned above, except that the Sertoli cells are isolated from Lewis male pups. Clear survival of allogeneic cells in the rat spinal cord is observed both 3 days and 15 days after implantation (FIGS. 14B and C).

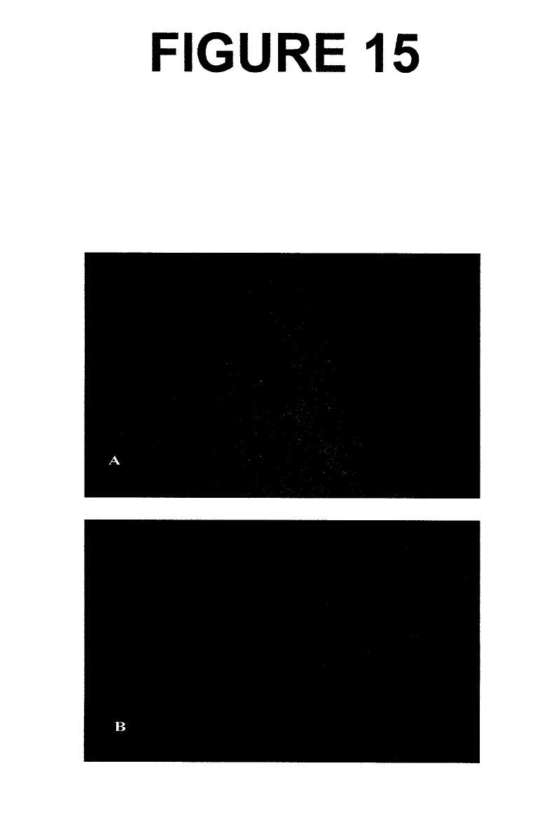

NT-3 secretion in vivo. The modified allogeneic cells when implanted into the spinal cord produce NT-3. Immunohistochemical analysis of spinal cord that is implanted with cells secreting NT-3 is shown in FIG. 15. Sections of the cord close to and away from the implantation site are used for analysis.

The protocol developed is as follows. The slides are hydrated in PBS and blocked in 2% rabbit serum/0.2% Triton X-100/0.1% BSA (RS/TX/BSA), for 5 min and then incubated in 10% rabbit serum/0.2% Triton X-100/0.1% BSA for 20 min. The primary Ab is Chicken anti-human NT-3 (0.5 mg/ml) from Promega (Madison, Wis.). NT-3 Ab is diluted 1:50 in 2% blocking buffer and slides are incubated with primary antibody at RT/overnight in a humid chamber. Slides are washed 3 times (5 min each) in PBS followed by incubation with secondary Ab that is rabbit anti chicken IgG-biotin conjugate (1 mg/ml) from Promega. Dilute the secondary Ab 1:100 (to a concentration of 10 ug/ml) in 2% RS-TX-BSA and incubate the slides with the secondary ab at RT/1 h. Wash with PBS, as described above and incubate with avidin conjugated dye, Alexa Fluor 350 (1:1000 dilution in PBS; Molecular Probes, Oreg.) at RT for 1 h. This is followed by three PBS washes. The slides are coverslipped with mounting medium (Vectashield, Vector Labs, Burlingame, Calif.). Fluorescence is evaluated at 200× magnification, using a Nikon Optiphot microscope equipped with an epi-fluorescence attachment (FIG. 15) in conjunction with SPOT 1 digital camera (Diagnostic Instruments, Sterling Heights, Mich.) and Photoshop 6.0 (Adobe, San Jose, Calif.).

Neurite growth assay. This is an in vitro biological assay to test the bioactivity of the NT-3 produced by the cells. The NT-3 produced by the cells is secreted into the medium, hence, supernatant from cells that are infected with Ad5CMV-NT-3-eGFP is collected and analyzed for biological activity as described below. This supernatant is also analyzed by ELISA, to determine the amount of NT-3 produced. As detected by ELISA and described above, the supernatant produced 37 ng of NT-3 per ml of culture medium in 24 h. Infected cells are implanted into injured rat spinal cord.

a: Embryonic neuronal cultures. Embryonic cortical neurons are isolated from mouse fetuses on embryonic day 16.5. These cells are cultured in vitro and the bioactivity of NT-3 secreted in the supernatant of the Sertoli cells infected with adenovirus is tested. Purified human NT-3 (BioVision, Mountain View, Calif.) and dehydroepiandrosterone (DHEA) are used as positive controls for the assay. Bioactivity is tested by measurements of the length of the neurites. The assay is performed as described by (Compagnone and Mellon, 1998). Cortical hemispheres are separated from the midbrain and hindbrain, and the basal ganglia are removed. After the removal of the hippocampus and the meninges, the cortical tissue is cut into small pieces and placed in PBS containing 0.03% collagenase and 1 □g/ml DNase I for 30 min at 37° C. After the incubation, a single cell suspension is made by mechanical trituration with a spinal needle and cells are filtered through a 40-□m nylon mesh. Cells are plated (50,000 cells per cm²) on glass coverslips coated with poly-D lysine (5 □g/cm², Roche) and 10% dextran and charcoal treated fetal bovine serum (Hyclone; Logan, Utah). The culture media is a modification of N2 serum-free medium used for culturing neuroblastoma cell lines (Bottenstein and Sato, 1979). The medium is DMEM-Ham F12 1:1 (2.24 g/liter bicarbonate, no phenol red) without serum, containing glucose (3.15 g/l), L-glutamine (2 mM), insulin (5 cig/ml, Roche), transferrin (5 □g/ml, Roche), selenium (3×10⁻⁸ M; Roche), putrescine (10⁻⁴ M; Sigma Chemical Co, St. Louis, Mo.), and lipids (0.5 □l/ml, GIBCO/BRL; Rockville, Md.). Cells are allowed to settle and attach to the coverslip for 2 h before the coverslip is inverted, as described (Lucius and Mentlein, 1995). Sandwiched cells are cultured for 3 days in 5% CO₂ at 37° C. and then treated according to the conditions described above. All the treatments are done in triplicates and the cells are treated for 16-20 h.

b: Immunocytochemistry. Following treatment cells are fixed for 20 min in 4% paraformaldehyde in 0.1 M PBS, pH 7.4. Fixed cells are preincubated for 1 h with 1× DIG (Roche) and BSA (2%) in PBS containing 0.03% Triton X-100 to block background immunostaining, and are then incubated overnight with the monoclonal Ab directed against bovine brain Tau-1 (Chemicon International, Temecula, Calif.) that is diluted 1:500 in the blocking buffer. Cells are than washed three times (5 min, each) in PBS and incubated with anti-mouse fluorescein isothiocyanate (FITC) conjugate (1:250 dilution in PBS) at RT for 1 h, washed in PBS as described above, mounted and observed under an epifluorescence microscope.

c: Morphometric Analysis. For each experiment a minimum of 33 neurons per well, three wells per treatment are randomly counted. Protocol for counting and measurement of neurite length is as described by Compagnone & Mellon, 1998 (Compagnone and Mellon, 1998). Immunopositive neurite length is determined at 250× magnification, using a Leitz Ortholux II microscope in conjunction with Optronix digital video cameras, Rasterops frame grabber application for Macintosh Power-PC 8500, and the NIH IMAGE 1.57 software. Scales are calibrated by using a microscope scale bar at the same magnification.

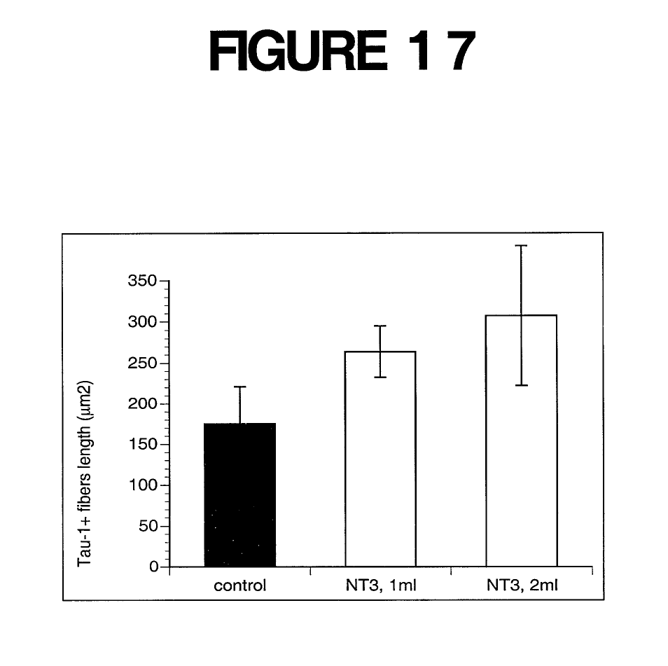

Neurons are isolated and treated as described above. Data shown is from one experiment only. In this analysis there is only 3 wells per treatment condition, and the culture medium did have 5.7% normal fetal bovine serum that can mask the effect of NT-3. Nonetheless a positive effect of NT-3 on neurite growth is observed. DHEA and recombinant NT-3 are used as positive controls and showed extensive axonal growth (data not shown). The very first analysis produced promising results indicating that the NT-3 produced and secreted by the infected Sertoli cells is bioactive (FIG. 17). Analysis of other experiments is ongoing.

Cell survival in injured spinal cord. Male Sprague Dawley rats, weighing 300-350 g, are used in this study. Animals are anesthetized with chloral hydrate (4.0%, 1 ml/100 g, i.p.). Rectal temperatures are maintained at 37° C. with a heating pad. A T8 laminectomy is performed. The spinal cord is injured by dropping a 10-g weight from a distance of 5 cm. This has been shown to induce a moderate level of injury (Noble and Wrathall, 1989). Control animals are only injured, and other animals are implanted with modified Sertoli cells expressing eGFP or eGFP and NT-3. Cell implantation is done as described above. Every 5^{th} section of the spinal cord is analyzed for the presence of labeled Sertoli cells.

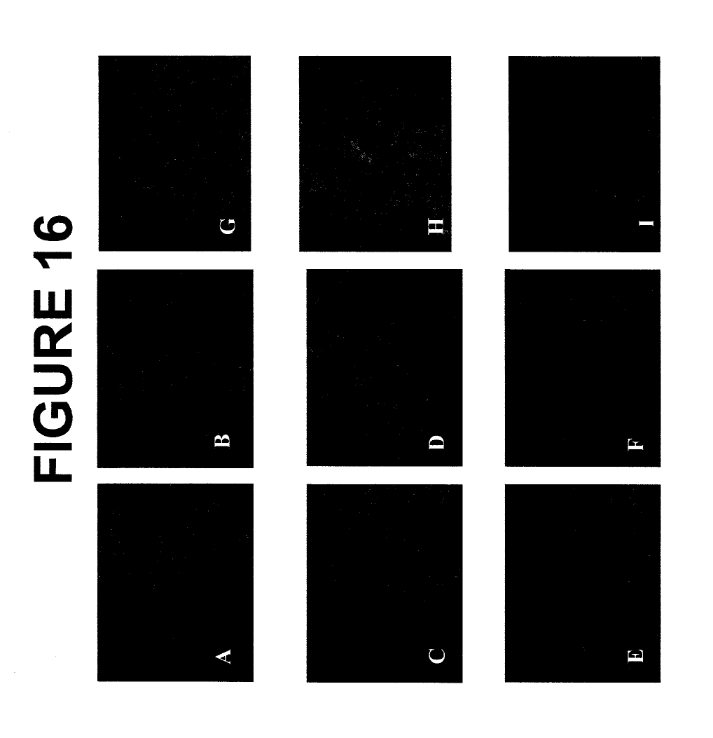

Immune response. Comparisons are made of the immune reactions in the spinal cord of animals 1) who were implanted with cells but not injured, 2) who were injured but did not implanted, and 3) who were injured and implanted. This is done qualitatively to look at the presence of macrophages and activated microglia. A monoclonal Ab to the complement C3bi receptor, OX42, is used to define microglia/macrophages. Sections are chosen that are proximal and distal to the site of injury and implantation, also sections that are obtained from implantation alone are analyzed. Slides are hydrated in PBS followed by incubation in blocking buffers, 2% goat serum/0.2% Triton X-100/0.1% BSA (GS/TX/BSA), 5 min; incubated in 10% goat serum/0.2% Triton X-100/0.1% BSA for 20 min. OX42 Ab (Serotec, Killington, OX, England) is diluted 1:3000 in 2% blocking buffer and slides are incubated with primary Ab at RT/overnight in a humid chamber. Slides are washed three times (5 min each) in PBS followed by incubation with biotinylated goat antimouse IgG (1:200 in GS/TX/BSA) at RT/1 h. Wash with PBS, as described above and incubate with avidin conjugate dye, Alexa Flour 350 (1:1000 dilution in PBS; Molecular Probes, Oreg.) at RT/1 h, again followed by three PBS washes and coverslipped with mounting medium (Vectashield, Vector Labs, Burlingame, Calif.). Fluorescence is evaluated at 200× magnification, using a Nikon Optiphot microscope equipped with an epi-fluorescence attachment (FIG. 16) in conjunction with SPOT 1 digital camera (Diagnostic Instruments, Sterling Heights, Mich.) and Photoshop 6.0 (Adobe, San Jose, Calif.).

No macrophages or activated microglia are observed in the uninjured animal that had received cell implants. Macrophages are observed in injured animal (3 days post-injury), close to the site of injury, and there are activated microglia away from the injury site. In injured animals that also received the cell implants, at 3 days post-implantation there are macrophages in both control and implanted spinal cords, but at 8 days post-implantation there are activated microglia only in the control animal (see FIG. 16). In the animal that received a cell implant, no macrophages or microglia are observed close to the injury site (data not shown) but a few activated microglia are observed away from the site of injury, but this experiment is only with one animal. More animals must be tested to determine if there is a decrease in the number of macrophages as a result of implantation of Sertoli cells that express NT-3. The other question addressed is whether the green fluorescing cells are in fact Sertoli cells and not macrophages that are green fluorescing because they have engulfed Sertoli cells (Geoffroy et al., 2000; Lenz et al., 2000). FIG. 16 (G, H, and I) illustrate fluorescent pictures of the same field of an injured rat spinal cord that is implanted with GFP expressing Sertoli cells and that is analyzed 3 days post injury. Even though there are macrophages present close to the implanted Sertoli cells, we clearly can distinguish the Sertoli cells in the field with an overlay of the OX42 stained blue image with the GFP image (I). This data demonstrates the potential of this methodology for sustained delivery of biologically active protein in the central nervous system, a location that is particularly difficult to access.

Functional Testing. This is the final test that determines the actual effect of NT-3 secreted by the implanted cells on the locomotor movements of the injured animals.

a: Locomotor movements. Weekly assessments of locomotion are performed for 6 weeks using the 21-point Basso, Beattie, and Bresnahan (BBB) rating scale (Basso et al., 1995). Initial points are avoided for isolated joint movements. Absence of observable hindlimb movements are scored 0, while slight movements of one or two joints are scored 1. Additional points are awarded for movement in more joints or more extensive movements. As motor performance increases, points are given for planter placement of the paw, stepping the forelimb-hindlimb coordination. The final points are achieved by toe clearance, trunk stability and tail position. Two observers will perform blinded open field-testing of rats for 5 min intervals. All hindlimb movements are recorded and scored. A maximum of 21 points are given per side for a possible total of 42.

b: Footprint Analysis. Once the animals initiate coordinated movements, they are tested for their footprints. Paw pads are dipped in non-toxic ink and they ambulate on white paper. This test shows how widespread the feet are and the paw placement during walking. Each footprint consists of paired footprint pads with five toe prints. A total of 10 footprints are examined from the final day of testing, using sets of footprints containing at least three consecutive strides. The following measurements are made: 1) stride length—the distance between footpads on two consecutive footprints; 2) base of support—distance between right and left foot; and 3) angle of rotation—the angle of intersection between lines defined by the angle of the footpad and toes, drawn according to standardized criteria (Kunkel-Bagden et al., 1993). Ten samples from each subject are analyzed, and individual subject means are determined.

### EXAMPLE 10

Isolation and culture of RPE and Sertoli cells from Callithrix jacchus marmoset. Multiple sclerosis (MS) is a spontaneous inflammatory demyelinating disease of the central nervous system that is mediated by an immune attack against myelin constituents. Recent research has shown that central nervous system expression of IL-10 using replication-deficient adenovirus can completely inhibit disease in the mouse model of EAE (Cua et al., 2001), in contrast to peripheral, intravenous administration of this cytokine (Cannella et al., 1996). These data indicate that immunomodulatory therapies for MS may have value if delivered specifically into the brain.

The C. jacchus marmoset experimental allergic encephalomyelitis (EAE) model for multiple sclerosis model (Genain and Hauser, 1997) has greater similarity to human MS than rodent models of acute EAE and is an ideal system to test future gene-based therapeutic strategies, because of evolutionary similarity between C. jacchus and humans. To study the delivery of therapeutic proteins in the brain in the C. jacchus EAE model RPE and Sertoli cells are isolated. The procedure used for isolation of Sertoli cells from the marmoset is the same as that described above for rat cells. The following procedure is used to isolate the RPE cells from the marmoset, as well as the rat and mouse (Sakagami et al., 1995). Immunocytochemical staining with cytokeratin-18 using a mouse monoclonal Ab (RGE53 clone, Chemicon International, Inc. Temecula, Calif.) can be used to determine the purity of RPE cell cultures.

A C. jacchus marmoset a few days old that was rejected by the mother died, and the eyes and surrounding connective tissues are removed. The eyes are dipped in 70% ethanol to sterilize them, and then rinsed once in Hanks' balanced salt solution (HBSS). Next the eyes are incubated in 0.1% proteinase K solution in HBSS for 15 min at 37° C. Then the eyeballs are rinsed once again in HBSS and placed in a dish containing HBSS. Under a steroscopic microscope, a circumferential incision is made just below the ora serrata of each eye. The anterior segment and the vitreous are removed and discarded and the eye placed in retinal pigment epithelial (RPE) growth medium (50:50 DMEM:F-12, 10% serum with antibiotics). Once all eye segments have been isolated the dish is placed at 37° C. for 20 min. Using the dissecting scope and a small “spoon” the RPEs are gently scrapped off the retina leaving behind the retina and remaining parts of the eye. Each scraped segment is removed from the dish along with any bits if tissue (muscle, fat, etc.) that may have been present leaving behind only the RPEs in medium. The RPEs are placed in a 15-ml conical vial and centrifuged at 2000 RPM for 5 min and the supernatant discarded. Then they are washed in calcium- and magnesium free HBSS 3 times repeating the centrifugation each time. The RPE are incubated in 1 ml of trypsin (0.25% in saline) for 15 min at 37° C., then 1 ml of trypsin inhibitor (0.34 g in 100 mls HBSS; Sigma Chemical Co., St. Louis, Mo.) and the cells are centrifuged at 2000 RPM for 5 min. The supernatant is discarded and the single RPE cells are suspended in RPE growth medium and counted with a hemocytometer. Approximately 5×10⁶ RPE cells are isolated from a single marmoset and these are plated in a single T-25 cm² flask. The cultured marmoset RPE cells have been passaged 5 times and apparently could continue to be passaged. In addition, the cells have been frozen and then thawed and cultured again successfully.

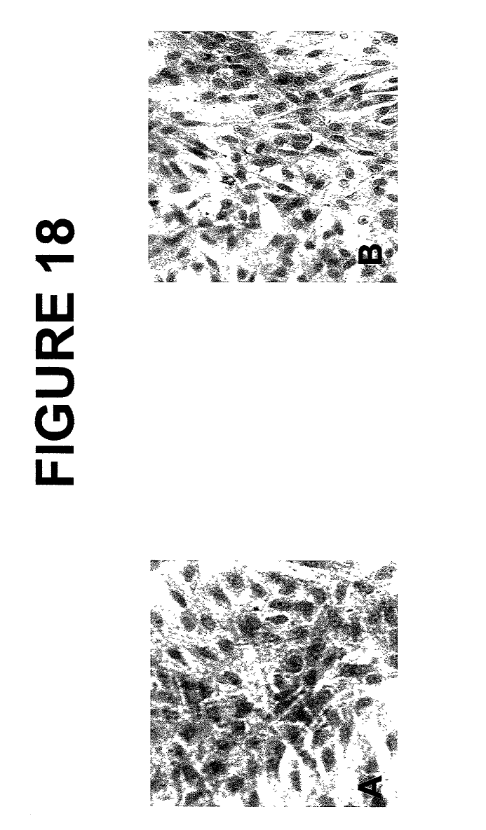

Microscopic analysis of RPE cells. RPE cells are grown in 8-well plates for microscopic analysis. Briefly, 1-2×10⁴ cells are plated per well of an 8-well Nalge Nunc Lab-Tek Chamber Slide with cover and grown in a CO₂ incubator at 37° C. for 2 days. The wells are washed twice with PBS at 37° C. for 2 min each time, and then twice more with PBS at RT for 2 min. The cells are fixed with 50:50 acetone:ethanol for 10 min at 4° C. for 10 min and the solution aspirated and the cells allowed to air day. The sample is stored in a 50:50 mix of PBS:glycerol 4° C., and washed again with PBS just prior to staining with hematoxylin-eosin (H & E). Microscopic analysis of the cells is performed at the Laboratory for Cell Analysis of the University of California Cancer Center (see FIGS. 18A and B).

### EXAMPLE 11

Determination of the relative immunosuppressive capacity of immune-privileged cells from mice. In this example in vitro assays are used to determine the relative immunosuppressive ability of cells to determine which type would be most suited from an immunological viewpoint for allogeneic implantation and delivery of proteins and genes in vivo. The isolation of the murine trophoblast progenitor stem cells and spleen cells is described below. Isolation of murine RPE and Sertoli cells is according to the procedure described above for the rat or marmoset.

A number of reports have shown immunosuppressive activity of one type of immune-privileged cells or cell culture supernatants but there has been little if any comparative analyses. Apoptosis of Jurkat T lymphocytes ranging from 42 to 83% was induced by human trophoblasts compared to induction of only 3 to 20% apoptosis in the control cells (Coumans et al., 1999). Additionally, the immunosuppressive cytokine IL-10 is found in the supernatant of primary cultures of human trophoblast cells and the secreted IL10 could inhibit allogeneic lymphocyte reactivity in vitro (Roth et al., 1996). Human fetal RPE cells suppressed the cell division of the human Jarkat T-lymphocyte cells and induced apoptosis as well (Farrokh-Siar et al., 2000). Explants of cornea or iris and ciliary body from normal eyes can suppress a mixed lymphocyte reaction (Streilein et al., 1996).

Isolation and establishment of trophoblast progenitor stem cells: In mammals the trophoblast cell lineage is specified before implantation. In mice, this lineage appears at the blastocyst stage as the trophectoderm, a sphere of epithelial cells surrounding the inner cell mass (ICM) and the blastocoel. It has been shown (Tanaka et al., 1998) that in the presence of FGF4 in culture most trophoblast cells are stem cells, whereas in the absence of FGF4 they differentiate into giant cells with multiple nuclei. Even under optimal culture conditions, some giant cells consistently appear at the edges of the colonies after each passage suggesting that a small percentage of the cells undergo differentiation (Tanaka et al., 1998). Clonal stem cell lines can be developed from each embryo. A distinct advantage of using progenitor stem cells for in vivo delivery of proteins and genes is that with stem cells a large number of cells can be produced in culture from a single donor. An ample and consistent supply of cells is important for reproducibility, safety, and cost of the eventual product.

Drs. Nathalie Rougier and Zena Werb, our collaborators from the University of California, San Francisco, can get 4-6 clones from 7 mouse embryos, and have maintained mouse TS cell lines for >50 passages over a period of more than 6 months with no apparent change in their morphology or viability.

The isolation protocol is as follows. The derivation of TS cell lines from 3.5 dpc mouse blastocysts is similar to the derivation of embryonic stem (ES) cell lines (Kuehn et al., 1987; Labosky et al., 1994; Tanaka et al., 1998). Briefly, matings are set up between mice of interest. Prepare 4-well plates of mitomycin-treated primary embryonic fibroblasts (EMFIs) in medium (RPMI 1640 that contains 20% fetal bovine serum, penicillin/streptomycin (5 □g/ml, each), sodium pyruvate (1 mM), beta-mercaptoethanol (100 □M) and L-glutamine (2 mM) the day before flushing. Replace TS medium with TS+F4H (FGF4, Sigma; and heparin) medium in the morning of the flushing day. Flush and collect 3.5 dpc blastocytes. In sterile conditions place one blastocyst per well in the 4-well plates containing TS+F4H medium and culture at 37° C./5%CO₂. The blastocytes should hatch and attach to the wells in 24-36 hrs. Feed culture with TS+F4H medium. Disaggregate the outgrowth on day 4 or 5 of culture as follows. Remove the medium and wash with PBS. Aspirate and add 0.1% trypsin/EDTA and incubate at 37° C./5%CO₂ for 5 min. Disaggregate the clump by pipetting up and down gently. Immediately stop the trypsinization by adding 70% conditioned medium (TS medium harvested from mitomycin C treated EMFIs)+30% TS medium+1.5× F4H. Change the medium 8 hr after disaggregation. Feed cells regularly and passage half-confluent well of TS cells to a regular 6-well plate or 35mm dish or into plates that contain mitomycin C treated MEFs. Most of the cells are frozen after the first passage, the remaining are used for the studies. As described below, cell morphology is the guideline for the number of the times the cells are passaged in culture. In culture normally three cell types are seen. Stem cells are the least differentiated and form the major population of the culture. Intermediate and giant cells are the differentiated forms of the stem cells and form a minor population of the total culture. Cells are cultured in vitro only till most of them are undifferentiated.

Isolation of primary spleen cells. Briefly, each mouse is anesthetized and sacrificed. The surrounding tissue is dissected with forceps and the spleen gently removed from each animal, rinsed with 70% ethanol and placed on a tissue culture dish. The spleen is covered with PBS and cut with a scissors into many small pieces. Spleen cells are isolated in cold PBS by repeated aspiration with a syringe and then filtration through a 70-micron sterile cell nylon strainer. The cells are centrifuged for 7-10 min at 300×g and the supernatant removed. The red blood cells are lysed by incubation of 3×10⁸ cell per ml for 10 min at RT in isotonic ammonium chloride solution pH 7.2 (9 vols of NH₄Cl-0.83% w/v in water with 1 vol of Tris-2.06% w/v in water and pH 7.65) sterilized by membrane filtration. Then the cells are washed by dilution with more than 5-fold excess PBS and mixed well. The cells are centrifuged for 7-10 min at 300×g and the supernatant removed and RPMI 1640 media with 5% fetal bovine serum, 5 mM glutamine, and penicillin/streptomycin antibiotics added.

Alternately instead of using isotonic ammonium chloride solution spleen cells are purified using Ficoll-Paque Plus (Amersham Pharmacia Biotech, Piscataway, N.J.). Half of the cell suspension is carefully overlaid on half volume of Ficoll-Paque in each of 2 15-ml centifuge tubes. The cells are spun at 400×g for 30 min at 20° C. with slow acceleration. The top of the four layers is plasma and is removed without disturbing other layers. Then the second layer containing the lymphocytes is removed and is transferred to warm RPMI media in a tube. The third layer is Ficoll-Paque, and bottom layer contains the red blood cells. The spleen cells are spun at 100×g for 10 min, the supernatant removed and then resuspended in media, and spun again at 100×g for 10 min. The media is removed and the cells suspended in RPMI media.

Treatment of effectors with mitomycin. To prevent proliferation the effector cells are treated with a solution of mitomycin C (10 □g/ml in culture media) at 37° C. for 2 h. Then the cells are washed 3 times with PBS to remove the mitomycin C. The trophoblast cell cultures are trypsinized, and washed. The effector cells are counted, and resuspended in RPMI 1640 media as described above (6,000 cells in 100 □l of RPMI 1640 media, 6×10⁴ cells/ml). Then 100 □l is added per well of a 96-well flat-bottomed microtiter plate, and the plate incubated in a CO₂ incubator at 37° C. for 4 hours.

ELISA Assay for cytotoxicity: An enzyme-linked immunosorbent assay (ELISA) is performed to compare the potential of trophoblast progenitor stem cells and Sertoli cells from inbred strain 129 mice to inflict cell-mediated cytotoxicity on allogeneic spleenocytes from CD1 mice. Other immune-privileged cell types such as RPE cells are also compared and other histoincompatible spleen cells also can be used. A photometric ELISA kit (Cellular DNA Fragmentation ELISA; Roche, Indianapolis, Ind.). for detection of 5-bromo-2deoxyuridine (BrdU)-labeled DNA fragments in culture supernatants and cell lysates is usd to determine the amount of BrdU labeled DNA released into the cell culture media by late-stage apoptotic or necrotic processes. Cells of the immune system such as cytotoxic T lymphocytes (CTLs), natural killer cells (NKs), and lymphokine-activated killer cells (LAKs) can recognize and destroy target cells, thus, allogeneic 129 spleenocytes are used as a population of allogeneic cells as a positive control. Syngeneic CD1 spleen cells are used as negative control.

The CD1 responder spleen cells are labeled with BrdU according to the instructions of the vendor. Briefly, the cells are adjusted to 2×10⁵ cells/ml of culture medium. Next, the BrdU labeling solution from the kit is added to the cells to a final concentration of 10 □m, and the cells incubated for 15 hours at 37° C. The cells are centrifuged at 250×g for 10 min. The supernatant is carefully removed and the cells are suspended in BrdU-free RPMI culture media. Then 10,000 spleen responder cells labeled with BrdU are added in 100 □l in RPMI media (1×10⁵ cells/ml) per well of a 96-well microtiter plate containing 6,000 mitomycin C treated effector cells per well and the plate is incubated for 6 hours in incubator at 37° C. To 4 wells the responder cells are added as described to wells containing media without effector cells and as a positive control an aliquot of dexamethasone is added to achieve a final concentration of 25 nm. Dexamethasone induces apoptosis in T lymphocytes with an ED₅₀ of 10 nM (Perandones et al., 1993). Responder cells are also added to 8 wells containing only supernatant from the progenitor trophoblast or Sertoli cell cultures. The microtiter plate is centrifuged at 300×g for 10 min and 100 □l of supernatant is carefully removed from each well and analyzed for DNA labeled with BrdU by ELISA. The detection of BrdU labeled DNA in the supernatant is indicative of the release of DNA fragments from damaged target cells and is reflective of cell-mediated cytotoxicity

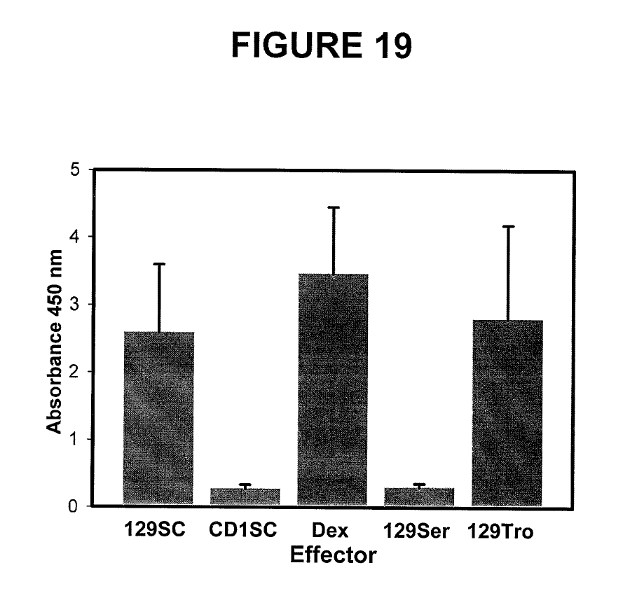

The results (see FIG. 19) indicate that the trophoblast progenitor cells are inducing more cell death than Sertoli cells in allogeneic spleen cells. The data support the immunosuppressive characteristic of trophoblast cells in general and specifically trophoblast progenitor cells compared to Sertoli cells. The data indicate that trophoblast cells will be more successful in defending themselves from attack by the immune system of the host and, therefore, better able to survive allogeneic implantation. This could be particularly important in regions of the body outside of the central nervous system that is partially protected from the immune system.

Proliferation Assay: Spleen cells from Balb/c and histoincompatible 129 male mice of 8 to 10 weeks of age are isolated as described above. Effector cells (allogeneic 129 spleen cells, trophoblast progenitor cells, RPE cells, or syngeneic Balb/c spleen cells are treated with mitomycin C in cell culture media to prevent proliferation as described above (10 □g/mI, 2 hr, 37° C.). The mitomycin C is removed by washing the cells 2 times with PBS and once with cell culture medium. To each well of a 96-well microtiter plate is added 30,000 effector cells in 100 □l of cell culture media. To each well is also added 100,000 responder Balb/c spleen cells. The microtiter plate is incubated for 72 h in 5% CO₂ at 37° C. Alternately, 30,000 regulator immune privileged cells that have been treated with mitomycin are added to mixed lymphocyte reactions (MLR) containing 100,000 responder Balb/c spleen cells and 30,000 histoincompatible effector 129 spleen cells. After 72 h BrdU is added to each well (10 □M) according to the vendor's instructions (Cell Proliferation ELISA BrdU—calorimetric; Roche) and the plate incubated for another 12 to 24 h in 5% CO₂ at 37° C. The microtiter plate is centrifuged at 300×g for 10 min and the labeling medium is carefully removed, and the plate dried at 60° C. for one hour, the cells fixed and the DNA denatured. The ELISA is carried out according to the instructions provided using an enzyme conjugated Ab to BrdU, and the color detected at 450 nm with a microplate reader.

### EXAMPLE 12

Comparative In Vivo Evaluation of Allogeneic Immune Privileged Cell Implants in Rat Kidney Capsule. To determine the best cell type for allogeneic implantation and in vivo delivery of recombinant genes or proteins comparative analyses are performed with implantations of different types of immune privileged cells as shown in Table 4. Further studies are to be performed with other cell types. Implantation into the kidney capsule is a standard procedure in studies of immune response to implanted cells. The immune response to implanted cells will vary somewhat depending on the site of implantation. An example of this is the difference in the immune reaction to foreign cells implanted in the central nervous system compared to the rest of the body. In addition to comparing the success of implantations at different sites, it is also important to compare different types of cells, cells that have been genetically modified with those that have not been modified, and the success of repeated cell implants.

In this example rat Sertoli and retinal pigement epithelial cells (RPE) are isolated as described above and implanted both allogeneically and syngeneically into Wistar-Furth rats. The survival of the cells and the immune response is assessed at the end of the experiment. Implantations are performed for either 3 or 14 days, and for 28 days. The immune response is characterized from the H & E stained slides in terms of the degree of subcapsular inflammation at the interface of renal tissue and capsule; 2) the presence of acute (with neutrophils) or chronic (mononuclear) inflammation; 3) the degree of capsule thickening (mild, moderate) with granulation tissue, and; 4) the presence of capsular fibrosis.

TABLE 4
Overview of Comparative Implants of in Wistar-Furth Rats

| DONOR CELLS | TYPE OF GRAFT | TIME OF GRAFT | NUMBER |
|---|---|---|---|
| Lewis Sertoli | allogeneic | 3 | 4 |
| | | 14 | 4 |
| Lewis Spleen | allogeneic | 3 | 1 |
| cells | | 14 | 2 |
| Lewis RPE | allogeneic | 3 | 4 |
| | | 14 | 4 |
| WF Sertoli | syngeneic | 3 | 2 |
| | | 14 | 2 |
| WF RPE | syngeneic | 14 | 2 |

Preparation of rat spleen cells. Rats are anesthetized with a mixture of ketamine/xylazine (66 mg/kg, 6.6 mg/kg, respectively) given intramuscularly. After the anesthetic takes effect a midline incision is made extending to the posterior to expose the abdominal cavity. After locating the spleen, the surrounding tissue is dissected with forceps, and the spleen gently removed and rinsed with 70% ethanol. The animal is sacrificed by cervical dislocation. The spleen is placed in a cell culture dish on ice and accessory tissue removed. Cold PBS is added to the dish and the spleen is cut into small pieces. The spleen cells are isolated by repeated aspiration in cold PBS using a syringe with an 18-gauge needle. The resulting cell suspension are filtered through several layers of sterile surgical gauze to remove large clumps, and then through a 70-micron sterile cell nylon. The cell culture dish is rinsed with cold PBS and the rinse liquid filtered also. The suspension is centrifuged for 7-10 min at 300×g in a 15-ml centrifuge tube. Then it is diluted to 10 to 15 ml and aspirated with pipette to distribute the cells evenly. Clumps that will not disperse are discarded. Centrifugation and wash step are repeated as needed up to 3 times. The cells are cultured in RPMI 1640 with 5% rat or fetal bovine serum and penicillin/streptomycin antibiotics at 37° C. in 5% CO₂. To count viable cells 10 □l of cell suspension at 10⁷ cells/ml are mixed with 10 □l of Trypan blue solution (0.2% of Trypan blue in PBS wth 3 mM NaN₃) in a small tube and resuspended with a pipette tip. The cells are examined in a hemacytometer chamber scoring more than 100 cells as to their state of viability within 5 min. Blue cells are dead and unstained are live. The red blood cells are lysed in isotonic ammonium chloride solution as described in example 11 above.

Cells Preparation for Transplantation. Frozen Sertoli and RPE cells are used. They are thawed in water bath at 37° C., and transferred into T-25 flask containing 5-ml medium, then incubated at 37° C. for 2-4 days. The spleen cells are prepared freshly. All the cells are washed 3 times with PBS (5 ml). To the Sertoli and RPE cells 2.5 ml of trypsin is added and the cells are set on the heating pad for 5 minutes. Next, 2.5 ml of serumcontaining medium is added to block the trypsin, and the cells are transferred to a 15-ml conical tube; and the cells are centrifuged at 2500 RPM. The medium is aspirated, and the cell pellet drawn into a glass Hamilton syringe that is preloaded with saline solution. A silicone tube adapter is placed over the syringe tip. PE50 tubing is inserted into the adapter and the cells are slowly injected into the PE50 tubing. A kink in the end of the tubing is made. While the kink is being maintained, the tubing is disconnected from the syringe, and then secured with the silicone tube adapter. The cells are placed in the PE50 tubing, and slid down the kinked silicone adapter into a 15-ml conical tube that is then centrifuged at 2500 rpm. The cells are left in the tubing until ready to implant.

Preparation of Rats for Transplantation. Each rat is injected a mixture of ketamine/xylazine (66 mg/kg, 6.6 mg/kg, respectively) intramuscularly. After anesthetic had taken effect, the left flank of the rat is shaved, and skin is swabbed with povidone iodine and ethanol. The left kidney is located; small incisions are made in the skin and the peritoneum to expose the kidney. Slight pressure is applied to both sides of the incision; the kidney is raised out of the rat abdomen. By using a cotton-tipped swab, the kidney is moistened with sterile saline. To create a nick in the kidney capsule, a small scratch on the right flank of the kidney is made by using a 23-gauge needle.

Cell Transplantation. The silicone adapter from the PE50 tubing is slowly removed while keeping the kink in the tubing. The opposite end of the PE50 tubing is connected to the silicone adapter and the silicone adapter is placed onto the tip of the glass syringe. Then the kink is slowly released, and the cells are slowly advanced to the tip of the PE50 tubing by using the “screw” mechanism. The PE50 tubing is carefully slid into the nick that is made in the kidney, a small pocket is created by gently moving the tubing in all directions, being careful not to gauge the kidney or puncture through the capsule. The cells are slowly advanced into “pocket” until all cells are transferred. The PE50 tubing is gently removed and the nick is carefully cauterized. All bleeding is stopped with a cotton-tipped swab. The kidney is re-moistened with sterile saline, and returned to the abdomen, prior to closing the incisions of the peritoneum and the skin. The rat is on a heating pad until it is fully active, and then transferred to its cage.

Kidney harvest, tissue preparation and histological staining. Each rat is euthanized by injecting with an overdose of ketamine/xylazine, followed by cervical dislocation. The kidney is excised, and placed in 10% formalin at 4° C. for histological analysis. A small piece of the kidney, about 5 mm³ in size, close to the visually observed implantation site is cut, and paraffin-embedded. Paraffin-embedded tissue blocks are serially sectioned in 5-micron wide sections, with 5 sections per slide. A total of 20-31 slides for each sample are further analyzed. The initial (#1), middle (#9-#12) or (#16-#22), and end (#20-#31) slides are selected and stained with hematoxylin-eosin (H & E) at the Pathology Laboratory at the Veterans Affairs Medical Center, San Francisco. A pathologist analyzes the slides for inflammatory and immune reactions. Immunocytochemistry is performed (Shi et al., 1991) with antibodies to follicle stimulating hormone receptor (FSHr) that is expressed by Sertoli cells, and to cytokeratin 18 that is expressed by RPE cells to assess the survival of the cells in the grafts.

### REFERENCES

1. Agrid, G., F. Javoy-Agrid, and M. Ruberg. 1987. Biochemistry of neurotransmitters in Parkinson's disease. In Movement Disorders. C. M. S. Fahn, editor. Butterworth's, London. 166-230.

2. Akahani, S., P. Nangia-Makker, H. Inohara, H. R. Kim, and A. Raz. 1997. Galectin-3: a novel antiapoptotic molecule with a functional BH1 (NWGR) domain of Bcl-2 family. Cancer Res. 57:5272-5276.

3. Al-Hendy, A., G. Hortelano, G. S. Tannenbaum, and P. L. Chang. 1995. Correction of the Growth Defect in Dwarf Mice with Nonautologous Microencapsulated Myoblasts—An Alternate Approach to Somatic Gene Therapy. Human Gene Therapy. 6:165-175.

4. Allen, H., D. Sucato, B. Woynarowska, S. Gottstine, A. Sharma, and R. Bemacki. 1990. Role of galaptin in ovarian carcinoma adhesion to 5. extracellular matrix in vitro. J Cell Biochem. 43:43-57.

6. Allison, J., H. M. Georgiou, A. Strasser, and D. L. Vaux. 1997. Transgenic expression of CD95 ligand on islet □ cells induces a granulocytic infiltration but does not confer immune privilege upon islet allografts. Proceedings of the National Academy of Sciences, United States of America. 94:3943-3947.

7. Anderson, R. D., R. E. Haskell, H. Xia, B. J. Roessler, and B. L. Davidson. 2000. A simple method for the rapid generation of recombinant adenovirus vectors. Gene Ther. 7:1034-1038.

8. Anderson, W. F., R. M. Blaese, and S. A. Rosenberg. 1995. U.S. Pat. No. 5,399,346.

9. Anel, A., M. Bufeme, C. Boyer, A. M. Schmitt-Verhulst, and P. Golstein. 1994. T cell receptor-induced Fas ligand expression in cytotoxic T lymphocyte clones is blocked by protein tyrosine kinase inhibitors and cyclosporin A. Eur. J Immunol. 24:2469-2476.

10. Anel, A., A. K. Simon, N. Auphan, M. Buferne, C. Boyer, P. Golstein, and A. M. Schmitt-Verhulst. 1995. Two signaling pathways can lead to Fas ligand expression in CD8+ cytotoxic T lymphocyte clones. Eur J Immunol. 25:3381-3387.

11. Arase, H., N. Arase, Y. Kobayashi, Y. Nishimura, S. Yonehara, and K. Onoe. 1994. Cytotoxicity of fresh NK1.1+ T cell receptor \tilde{□} □thymocytes against a CD4+8+ thymocyte population associated with intact Fas antigen expression on the target. J Exp Med. 180:423-432.

12. Arase, H., N. Arase, and T. Saito. 1995. Fas-mediated cytotoxicity by freshly isolated natural killer cells. J. Exp. Med. 181:1235-1238.

13. Archer, J. S., W. S. Kennan, M. N. Gould, and R. D. Bremel. 1994. Human growth hormone (hGH) secretion in milk of goats after direct transfer of the hGH gene into the mammary gland by using replication-defective retrovirus vectors. Proc. Natl. Acad. Sci. USA. 91:6840-6844.

14. Archibald, A. L., A. J. Clark, S. Harris, M. McClenaghan, J. P. Simons, and C. B. Whitelaw. 1997. Genetic construct of which protein coding DNA comprises introns and is designed for protein production in transgenic animals. U.S. Pat. No. 5,650,503.

15. Ausubel, F. M., R. Brent, R. E. Kingdon, D. M. Moore, J. G. Seidman, J. A. Smith, and K. Struhl. 1994. Current Protocols in Molecular Biology. Greene Publishing Associates, Inc. and John Wiley and Sons, Inc.

16. Avallet, O., M. Vigier, P. Leduque, P. M. Dubois, and J. M. Saez. 1994. Expression and regulation of transforming growth factor-beta 1 messenger ribonucleic acid and protein in cultured porcine Leydig and Sertoli cells. Endocrinology. 134:2079-2087.

17. Azain, M. J., T. R. Kasser, M. J. Sabacky, and C. A. Baile. 1992. Comparison of the growth-promoting properties of daily vs continuous administration of somatotropin in female rats with intact pituitaries. J Anim Sci. 71:384-392.

18. Babensee, J., J. Anderson, L. McIntire, and A. Mikos. 1998. Host response to tissue engineered devices. Advanced Drug Delivery Reviews. 33:111-139.

19. Badley, A. D., J. A. McElhinny, P. J. Leibson, D. H. Lynch, M. R. Alderson, and C. V. Paya. 1996. Upregulation of Fas ligand expression by human immunodeficiency virus in human macrophages mediates apoptosis of uninfected T lymphocytes. J Virol. 70:199-206.

20. Barbeau, A. 1961. Biochemistry of Parkinson's disease. Int Congr Series. 38:152-153.

21. Barr, E., and J. M. Leiden. 1991. Systemic delivery of recombinant proteins by genetically modified myoblasts. Science. 254:1506-1509.

22. Bartek, J., J. Bartkova, N. Kyprianou, E. N. Lalani, Z. Staskova, M. Shearer, S. Chang, and J. Taylor-Papadimitriou. 1991. Efficient immortalization of luminal epithelial cells from human mammary gland by introduction of simian virus 40 large tumor antigen with a recombinant virus. Proceedings of the National Academy of Sciences, United States of America. 88:3520-3524.

23. Basso, D. M., M. S. Beattie, and J. C. Bresnahan. 1995. A sensitive and reliable locomotor rating scale for open field testing in rats. J Neurotrauma. 12:1-21.

24. Bell, G. I., W. F. Swain, R. Pictet, B. Cordell, H. M. Goodman, and W. J. Rutter. 1979. Nucleotide sequence of a cDNA clone encoding human preproinsulin. Nature. 282:525-527.

25. Beilgrau, D., D. Gold, H. Selawry, J. Moore, A. Franzusoff, and R. C. Duke. 1995. A role for CD95 ligand in preventing graft rejection. Nature. 377:630-632.

26. Bennett, J., J. Wilson, S. Dexue, B. Borgbes, and A. Maguire. 1994. Adenovirus vector-mediated in vivo gene transfer into adult murine retina. Invest Ophthamol Vis Sci. 35:2535-2542.

27. Bennett, W. A., S. Lagoo-Deenadayalan, N. S. Whitworth, M. N. Brackin, E. Hale, and B. D. Cowan. 1997. Expression and production of interleukin-10 by human trophoblast: relationship to pregnancy immunotolerance. Early Pregnancy. 3:190-198.

28. Bevilacqua, E. M., M. R. Faria, and P. A. Abrahamsohn. 1991. Growth of mouse ectoplacental cone cells in subcutaneous tissues. Development of placental-like cells. Am J Anat. 192:382-399.

29. Birkmayer, W., and O. Homykiewicz. 1961. Effekt bei der parkinson akinesia. Klin Wochenschr. 73:787-788.

30. Bloxam, D. L., C. M. Bax, and B. E. Bax. 1997. Culture of syncytiotrophoblast for the study of human placental transfer. Part I: Isolation and purification of cytotrophoblast. Placenta. 18:93-98.

31. Bottenstein, J. E., and G. H. Sato. 1979. Growth of a rat neuroblastoma cell line in serum-free supplemented medium. Proc Natl Acad Sci USA. 76:514-517.

32. Bottenstein, J. E., and G. H. Sato. 1980. Fibronectin and polylysine requirement for proliferation of neuroblastoma cells in defined medium. Exp Cell Res. 129:361-366.

33. Bouck, N., and G. DiMayorca. 1979. Evaluation of chemical carcinogenicity by in vitro neoplastic transformation. In Methods Enzymol. Vol. 58.296-302.

34. Bourdon, V., A. Lablack, P. Abbe, D. Segretain, and G. Pointis. 1998. Characterization of a clonal Sertoli cell line using adult PyLT transgenic mice. Biol Reprod. 58:591-599.

35. Bowen, J. A., and J. S. Hunt. 1999. Expression of cell adhesion molecules in murine placentas and a placental cell line. Biol Reprod. 60:428-434.

36. Bradbury, E. J., S. Khemani, R. Von, King, J. V. Priestley, and S.B. McMahon. 1999. NT-3 promotes growth of lesioned adult rat sensory axons ascending in the dorsal columns of the spinal cord. Eur J Neurosci. 11:3873-3883.

37. Brady, O. 1966. The sphingolipidoses. N Engl J Med. 275:312-318.

38. Bregman, B. S., E. Broude, M. McAtee, and M. S. Kelley. 1998. Transplants and neurotrophic factors prevent atrophy of mature CNS neurons after spinal cord injury. Exp Neurol. 149:13-27.

39. Bregman, B. S., M. McAtee, H. N. Dai, and P. L. Kuhn. 1997. Neurotrophic factors increase axonal growth after spinal cord injury and transplantation in the adult rat. Exp Neurol. 148:475-494.

40. Brown, O., J. R. McNeilly, R. M. Wallace, A. S. McNeilly, and A. J. Clark. 1993. Characterization of the ovine LH beta-subunit gene: the promoter directs gonadotrope-specific expression in transgenic mice. Mol Cell Endocrinology. 93:157-165.

41. Bruck, S. D. 1991. Objectives, Critical Reviews in Therapeutic Drug Carrier Systems.

42. Cameron, D. F., H. U. Wyss, L. J. Romrell, and F. T. Murray. 1987. Sertoli cells maintain spermatid viability in vitro. In Cell Biology of the Testis and Epididymis. D. B. Orgebin-Crist M C, editor. Annals of the New York Academy of Science, New York, N.Y. 419-423.

43. Cannella, B., Y. L. Gao, C. Brosnan, and C. S. Raine. 1996. IL-10 fails to abrogate experimental autoimmune encephalomyelitis. J Neurosci Res. 45:735-746.

44. Carosella, E. D. 2000. [HLA-G: fetomatemal tolerance]. C R Acad Sci III. 323:675-680.

45. Carosella, E. D., I. Khalil-Daher, J. Dausset, and N. Rouas-Freiss. 1999. HLA-G mediates protection from natural killer cytolysis: implications in immune tolerance. Transplant Proc. 31:1192-1193.

46. Catt, J., F. Harrison, and J. Carleton. 1987. Distribution of an endogenous beta-galactoside-specific lectin during foetal and neonatal rabbit development. J Cell Sci. 87:623-633.

47. Caussanel, V., E. Tabone, J. C. Hendrick, F. Dacheux, and M. Benahmed. 1997. Cellular distribution of transforming growth factor betas 1, 2, and 3 and their types I and II receptors during postnatal development and spermatogenesis in the boar testis. Biol Reprod. 56:357-367.

48. Charlton, H. M., R. G. Clark, I. C. A. F. Robinson, A. E. Porter Goff, B. S. Cox, C. Bugnon, and B. A. Bloch. 1988. Growth hormone-deficient dwarfism in the rat: a new mutation. J Endocrinology. 119:51-58.

49. Chaudhary, J., P. D. Whalley, A. Cupp, and M. K. Skinner. 1996. Transcriptional regulation of sertoli cell differentiation by follicle-stimulating hormore at the level of the c-fos and transferrin promoters. Biol Reprod. 54:692-699.

50. Chen, C., and H. Okayama. 1987. High-efficiency transformation of mammalian cells by plasmid DNA. Mol Cell Biol. 7:2745-2752.

51. Chen, J. J., Y. Sun, and G. J. Nabel. 1998. Regulation of the proinflammatory effects of Fas ligand (CD95L). Science. 282:1714-1717.

52. Cheng, C. Y. 1990. Purification of a calcium binding protein (rat SPARC) from primary Sertoli cell-enriched culture medium. Biochem Biophys Res Commun. 167:1393-1399.

53. Chervonsky, A. V., Y. Wang, F. S. Wong, I. Visintin, R. A. Flavell, J. Janeway, C A, and L. A. Matis. 1997. The role of Fas in autoimmune diabetes. Cell. 89:17-24.

54. Chomczynski, P., and N. Sacchi. 1987. Single-step method of RNA isolation by acid guanidium thiocyanate-phenol-chloroform extraction. Anal Biochem. 162:156-159.

55. Cibelli, J. B., S. L. Stice, P. J. Golueke, J. J. Kane, J. Jerry, C. Blackwell, F. A. deLeon, and J. M. Robl. 1998. Transgenic bovine chimeric offspring produced from somatic cell-derived stem-like cells. Nat Biotech. 16:642-646.

56. Clark, R. G., J. -O. Jansson, O. G. P. lsaksson, and A. C. A. F. Robinson. 1985. Intravenous growth hormone: growth responses to patterned infusions in hypophysectomized rats. J Endocrinol. 104:53-61.

57. Cohen, A. J., and C. M. Kessler. 1995. Treatment of Inherited Coagulation Disorders. Amer. J. Med. 99:675-682.

58. Compagnone, N. A., and S. H. Mellon. 1998. Dehydroepiandrosterone: a potential signalling molecule for neocortical organization during development. Proc Natl Acad Sci USA. 95:4678-4683.

59. Constantini, S., and W. Young. 1994. The effects of methylprednisolone and the ganglioside GM1 on acute spinal cord injury in rats. J Neurosurg. 80:97-111.

60. Coumans, B., O. Thellin, W. Zorzi, F. Melot, M. Bougoussa, L. Melen, D. Zorzi, G. Hennen, A. Igout, and E. Heinen. 1999. Lymphoid cell apoptosis induced by trophoblastic cells: a model of active foeto-placental tolerance. J Immunol Methods. 224:185-196.

61. Croy, B. A., J. Rossant, and D. A. Clark. 1984. Recruitment of cytotoxic cells by ectopic grafts of xenogeneic, but not allogeneic, trophoblast. Transplantation. 37:84-90 in the placenta and were always present in the grafts. Cells showing features intermediate between the above-mentioned cells and those whose cytoplasm was poor in organelles also were found in the grafts. The latter resembled cells of layer 81 of the labyrinth of the placenta. These results suggest that trophoblastic cells of the ectoplacental cones had differentiated into placental cells following their transfer to the subcutaneous tissue.

62. Cua, D. J., B. Hutchins, D. M. LaFace, S. A. Stohlman, and R. L. Coffman. 2001. Central nervous system expression of IL-10 inhibits autoimmune encephalomyelitis. J Immunol. 166:602-608.

63. Culver, K. W., Z. Ram, S. Wallbridge, H. Ishii, E. H. Oldfield, and R. M. Blase. 1992. In vivo gene transfer with retroviral vector-producer cells for treatment of experimental brain tumors. Science. 256:1550-1552.

64. Dahler, A., R. P. Wade, G. E. O. Muscat, and M. Waters. 1994. Expression vectors encoding human growth hormone (hGH) controlled by human muscle-specific promoters: prospects for regulated production of hGH delivered by myoblast transfer or intravenous injection. Gene. 145:305-310.

65. Dai, Y., M. Roma, R. K. Naviaux, and I. M. Verma. 1992. Gene therapy via primary myoblasts: Long-term expression of factor IX protein following transplantation in vivo. Proc. Natl. Acad. Sci. USA. 89:10892-10895.

66. Damak, S., H. Su, N. P. Jay, and D. W. Bullock. 1996. Improved wool produced in transgenic sheep expressing insulin-like growth factor 1. Biotechnology. 14:185-188.

67. Davidson, F. F., and H. Steller. 1998. Blocking apoptosis prevents blindness in Drosophila retinal degeneration mutants. Nature. 391:587-591.

68. Dean, M. F., H. Muir, P. F. Benson, L. R. Button, J. R. Batchelor, and M. Bewick. 1975. Increased breakdown of glycosaminoglycans and appearance of corrective enzyme after skin transplants in Hunter syndrome. Nature. 257:609-612.

69. Deboer, H. A., R. Strijker, H. L. Heyneker, G. Platenburg, S. H. Lee, F. Piper, and P. Krimpenfort. 1998. Transgenic bovine. U.S. P. No. 5,741,957.

70. DeNoto, F. M., D. D. Moore, and H. M. Goodman. 1981. Human growth hormone DNA sequence and mRNA structure: possible alternative splicing. NAR. 9:3719-3730.

71. Dhawan, J., L. C. Pan, G. H. Paviath, M. A. Travis, A. M. Lanctot, and H. M. Blau. 1991. Systemic delivery of human growth hormone by injection of genetically engineered myoblasts. Science. 254:1509-1511.

72. DiStefano, P. S., B. Friedman, C. Radziejewski, C. Alexander, P. Boland, C. M. Schick, R. M. Lindsay, and S. J. Wiegand. 1992. The neurotrophins BDNF, NT-3, and NGF display distinct patterns of retrograde axonal transport in peripheral and central neurons. Neuron. 8:983-993.

73. Domb, A., M. Maniar, S. Bogdansky, and M. Chasin. 1991. Drug Delivery to the Brain Using Polymers. Critical Reviews in Therapeutic Drug Carrier Systems,. 8:1-17.

74. Ducray, A., M. Bloquel, K. Hess, G. L. Hammond, H. Gerard, and A. Gerard. 1998. Establishment of a mouse Sertoli cell line producing rat androgen-binding protein (ABP). Steroids. 63:285-287.

75. Dunaief, J. L., R. C. Kwun, N. Bhardwaj, R. Lopez, P. Gouras, and S. P. Goff. 1995. Retroviral gene transfer into retinal pigment epithelial cells followed by transplantation into rat retina. Human Gene Therapy. 6:1225-1229.

76. Dunn, C. S., M. Mehtali, L. M. Houdebine, J. P. Gut, A. Kirn, and A. M. Aubertin. 1995. Human immunodeficiency virus type 1 infection of human CD4-transgenic rabbits. J Gen Virol. 76:1327-1336.

77. During, M. J., J. R. Naegele, K. L. O'Malley, and A. I. Geller. 1994. Long-term behavioral recovery in parkinsonian rats by an HSV vector expressin tyrosine hydroxylase. Science. 266:1399-1403.

78. Duverger, N., C. Viglietta, L. Berthou, F. Emmanuel, A. Tailleux, L. Parmentier-Nihoul, B. Laine, C. Fievet, G. Castro, J. C. Fruchart, L. M. Houdebine, and P. Denaefie. 1996. Transgenic rabbits expressin human apolipoprotein A-I in the liver. Arter Thromb Vascular Biol. 16:1424-1429.

79. Dwarki, V. J., P. Belloni, T. Nijjar, J. Smith, L. Couto, M. Rabier, S. Clift, A. Bems, and L. K. Cohen. 1995. Gene therapy for hemolphilia A: Production of therapeutic levels of human factor VIII in vivo in mice. Proc Natl Acad Sci USA. 92:1023-1027.

80. Ellis, S., I. Sargent, C. Redman, and A. McMichael. 1986. Evidence for a novel HLA antigen found on human extravillous trophoblast and a choriocarcinoma cell line. Immunol. 59:595-601.

81. Esser, P., S. Grisanti, N. Kociok, H. Abts, A. Hueber, K. Unfried, K. Heimann, and M. Weller. 1997. Expression and upregulation of microtubule-associated protein 1 B in cultured retinal pigment epithelial cells. Investig. Opthal. Vis. Sci. 38:2852-2856.

82. Evans, R., R. D. Palmiter, and R. L. Brinster. 1989. Method of obtaining gene product through the generation of transgenic animals. U.S. P. No. 4,870,009.

83. Fairhall, K. M., D. F. Carmignac, and I. C. A. F. Robinson. 1992. Growth hormone (GH) binding protein and GH interactions in vivo in the guinea pig. Endocrinology. 131:1963-1969.

84. Farrokh-Siar, L., K. A. Rezai, E. M. Palmer, S. C. Patel, J. T. Ernest, and G. A. van Seventer. 2000. Human fetal retinal pigment epithelium-induced cell cycle arrest, loss of mitochondrial membrane potential and apoptosis [In Process Citation]. Invest Ophthalmol Vis Sci. 41:3991-3998.

85. Finlay, D., G. Wilkinson, R. Kypta, I. de Curtis, and L. Reichardt. 1996. Retinal Cultures. Methods in Cell Biology. 51:265-283.

86. Flavell, D. M., T. Wells, S. E. Wells, D. F. Carmignac, G. B. Thomas, and I. C. A. F. Robinson. 1996. Dominant dwarfism in transgenic rats by targeting human growth hormone (GH) expression to hypothalamic GH-releasing factor neurons. EMBO J. 15:3871-3879.

87. Franklin, G. C., M. Donovan, G. I. Adam, L. Holmgren, S. Pfeifer-Ohlsson, and R. Ohisson. 1991. Expression of the human PDGF-B gene is regulated by both positively and negatively acting cell type-specific regulatory elements located in the first intron. EMBO J. 10:1365-1373.

88. Freese, A., M. G. Kaplitt, W. M. O'Connor, M. Abbey, D. Langer, P. Leone, M. J. O'Connor, and M. J. During. 1997. Direct gene transfer into human epileptogenic hippocampal tissue with an adeno-associated virus vector: implications for a gene therapy approach to epilepsy. Epilepsia. 38:759-766.

89. French, L. E., M. Hahne, I. Viard, G. Radlgruber, R. Zanone, K. Becker, C. Muller, and J. Tschopp. 1996. Fas and fas ligand in embryos and adult mice: Ligand expression in several immune-privileged tissues and coexpression in adult tissues characterized by apoptotic cell turnover. Journal of Cell Biology. 133:335-343.

90. Freshney, R. 1994. Culture of Animal Cells: a manual of basic technique. Alan R. Liss, New York.

91. Friedmann, T., Y. W. Kan, and R. Mulligan. 1994. Gene Therapy. In Gene Therapy. Cold Spring Harbor Laboratory, Cold Spring Harbor Laboratory.

92. Friedmann, T., and R. Toblin. 1972. Gene therapy for human genetic disease? Science. 175: 949-955.

93. Gage, F. H., T. Friedmann, M. B. Rosenberg, J. W. Wolff, M. D. Kawaja, and J. Ray. 1997. Method of grafting genetically modified cells to treat defects, disease of damage of the central nervous system. U.S. Pat. No. 5,650,148.

94. Galdieri, M., E. Ziparo, F. Palombi, M. A. Russo, and M. Stefanini. 1981. Pure Sertoli cell cultures: A new model of the study of somatic-germ cell interactions. J. Androl. 5:249-254.

95. Geerts, A., T. Niki, K. Hellemans, D. DeCraemer, K. Van Den Berg, J. M. Lazou, G. Stange, M. Van De Winkel, and P. DeBleser. 1997. Purification of rat hepatic stellate cells by side scatter-activation cell sorting. Hepatology. 27:590-598.

96. Genain, C. P., and S. L. Hauser. 1997. Creation of a model for multiple sclerosis in Callithrix jacchus marmosets. J Mol Med. 75:187-197.

97. Geoffroy, M. C., C. Guyard, B. Quatannens, S. Pavan, M. Lange, and A. Mercenier. 2000. Use of green fluorescent protein to tag lactic acid bacterium strains under development as live vaccine vectors. Appl Environ Microbiol. 66:383-391.

98. George, J. F., and J. M. Thomas. 1999. The molecular mechanisms of veto mediated regulation of alloresponsiveness. J Mol Med. 77:519-526.

99. Gershon, S. L., A. I. Caplan, and S. E. Haynesworth. 1997. Transduced mesenchymal stem cells. U.S. Pat. No. 5,591,625.

100. Ghattas, I. R., J. R. Sanes, and J. E. Majors. 1991. The encephalomyocarditis virus internal ribosome entry site allows efficient coexpression of two genes from a recombinant provirus in cultured cells and in embryos. Mol. Cell Biol. 11:5848-5849.

101. Gitschier, J., W. I. Wood, T. M. Goralka, K. L. Wion, E. Y. Chen, D. H. Eaton, G. A. Vehar, D. J. Capon, and R. M. Lawn. 1984. Characterization of the human factor VIII gene. Nature. 312:326-330.

102. Gjertson, D. W. 1991. Clinical Transplants. P. Terasaki, editor. UCLA Press, Los Angeles. 225.

103. Gordon, J. W., G. A. Scangos, D. J. Plotkin, J. A. Barbosa, and F. H. Ruddle. 1980. Genetic transformation of mouse embryos by microinjection of purified DNA. Proc. Natl. Acad. Sci. USA. 77:7380-7384.

104. Gotz, R., R. Kolbeck, F. Lottspeich, and Y. A. Barde. 1992. Production and characterization of recombinant mouse neurotrophin-3. Eur J Biochem. 204:745-749.

105. Green, D. R., and C. F. Ware. 1997. Fas-ligand: Privilege and peril. Proceedings of the National Academy of Sciences, United States of America. 94:5986-5990.

106. Griffith, T. S., T. Brunner, S. M. Fletcher, D. R. Green, and T. A. Ferguson. 1995. Fas Ligand-Induced Apoptosis as a Mechanism of Immune Privilege. Science. 270:1189-1192.

107. Grill, R., K. Murai, A. Blesch, F. H. Gage, and M. H. Tuszynski. 1997. Cellular delivery of neurotrophin-3 promotes corticospinal axonal growth and partial functional recovery after spinal cord injury. J Neurosci. 17:5560-5572.

108. Grima, B., A. Lamouroux, C. Boni, J. F. Julien, F. Javoy-Agid, and J. Mallet. 1987. A single human gene encoding multiple tyrosine hydroxylases with different predicted functional characteristics. Nature. 326:707-711.

109. Gros, L., L. Montoliu, E. Riu, L. Lebrigand, and F. Bosch. 1997. Regulated production of mature insulin by nono-B-cells. Human Gene Therapy. 8:2249-2259.

110. Groth, C. G., K. I. Bergstrom, L. Collste, N. Egberg, C. Hogman, G. Holm, and E. Moller. 1972. Immunologic and plasma protein studies in a splenic homograft recipient. Clin Exp Immunol. 10:359-365.

111. Gruner, J. A. 1992. A monitored contusion model of spinal cord injury in the rat. J Neurotrauma. 9:123-126; discussion 126-128.

112. Gupta, S., S. Masinick, M. Garrett, and L. Hazlett. 1997. Pseudomonas aeruginosa lipopolysaccharide binds galectin-3 and other human corneal epithelial proteins. Infect Immun. 65:2747-2753.

113. Hagihara, Y., Y. Saitoh, H. Iwata, T. Taki, S. Hirano, N. Arita, and T. Hayakawa. 1997. Transplantation of xenogeneic cells secreting beta-endorphin for pain treatment: analysis of the ability of components of complement to penetrate through polymer capsules. Cell Transplant. 6:527-530.

114. Hahn, T. M., K. C. Copeland, and S. L. C. Woo. 1996. Phenotypic correction of dwarfism by constiutive expression of growth hormone. Endocrinology. 137:4988-4993.

115. Hahne, M., M. C. Peitsch, M. Irmler, M. Schroter, B. Lowin, M. Rousseau, C. Bron, T. Renno, L. French, and J. Tschopp. 1995. Characterization of the non-functional Fas ligand of gid mice. Internatl Immunol. 7:1381-1386.

116. Hahne, M., T. Renno, M. Schroeter, M. Irmler, L. French, T. Bornard, H. R. MacDonald, and J. Tschopp. 1996. Activated B cells express functional Fas ligand. European Journal of Immunology. 26:721-724.

117. Hammer, A., H. Hutter, A. Blaschitz, W. Mahnert, M. Hartmann, B. Uchanska-Ziegler, A. Ziegler, and G. Dohr. 1997. Amnion epithelial cells, in contrast to trophoblast cells, express all classical HLA class I molecules together with HLA-G. Am J Reprod Immunol. 37:161-171.

118. Hanania, E. G., J. Kavanagh, G. Hortobagyi, R. E. Giles, R. Champlin, and A. B. Deisseroth. 1995. Recent Advances in the Application of Gene Therapy to Human Disease. Amer. J. of Med. 99:537-552.

119. Hancock, A. D., D. M. Robertson, and D. M. de Kretser. 1992. Inhibin and inhibin alpha-chain precursors are produced by immature rat Sertoli cells in culture. Biol Reprod. 46:155-161.

120. Handelsman, D. J., J. A. Spaliviero, and A. F. Phippard. 1990. Highly vectorial secretion of inhibin by primate Sertoli cells in vitro. J Clin Endocrin Metab. 71:1235-1238.

121. Harris, D. P., A. T. Andrews, G. Wright, D. L. Pyle, and J. A. Arsenjo. 1997. The application of aqueous two-phase systems to the purificatioin of pharmaceutical proteins from transgenic sheep. Bioseparation. 7:31-37.

122. Hayward, S. W., R. Dahiya, G. R. Cunha, J. Bartek, N. Deshpande, and P. Narayan. 1995. Establishment and characterization of an immortalized but non-transformed human prostate epithelial cell line: BPH-1. In Vitro Cellular and Developmental Biology. 31 A:14-24.

123. Heartlein, M. W., V. A. Roman, J. -L. Jiang, J. W. Sellers, A. M. Zullianai, D.A. Treco, and R. F. Selden. 1994. Long-term production and delivery of human growth hormone in vivo. Proc. Natl. Acad. Sci. USA. 91:10967-10971.

124. Henderson, C. E., W. Camu, C. Mettling, A. Gouin, K. Poulsen, M. Karihaloo, J. Rullamas, T. Evans, S. B. McMahon, M. P. Armanini, and et al. 1993. Neurotrophins promote motor neuron survival and are present in embryonic limb bud. Nature. 363:266-270.

125. Hennessy, A., H. L. Pilmore, L. A. Simmons, and D. M. Painter. 1999. A deficiency of placental IL-10 in preeclampsia. J Immunol. 163:3491-3495.

126. Herbertson, A., and J. E. Aubin. 1997. Cell sorting enriches osteogenic populations in rat bone marrow stromal cell cultures. Bone. 21:491-500.

127. Hirabayashi, J., and K. Kasai. 1984. Human placenta beta-galactoside-binding lectin. Purification and some properties. Biochem Biophys Res Commun. 122:938-944.

128. Hogan, B., Constantini, F, Lacy, E. 1986. Manipulating the Mouse Embryo. Cold Spring Harbor Laboratory Press, Cold Spring Harbor, N.Y.

129. Hohn, A., J. Leibrock, K. Bailey, and Y. A. Barde. 1990. Identification and characterization of a novel member of the nerve growth factor/brain-derived neurotrophic factor family. Nature. 344:339-341.

130. Horellou, P., E. Vigne, M. N. Castel, P. Barneoud, P. Colin, M. Perricaudet, P. Delaere, and J. Mallet. 1994. Direct intracerebral gene transfer of an adenoviral vector tyrosine hydroxylase in a rat model of Parkinson's disease. Neuroreport. 6:49-53.

131. Hori, J., N. Joyce, and J. W. Streilein. 2000. Epithelium-deficient corneal allografts display immune privilege beneath the kidney capsule. Invest Ophthalmol Vis Sci. 41:443-452.

132. Houdebine, L. -M. 1994. Production of pharmaceutical proteins from transgenic animals. J. Biotechnol. 34:269-287.

133. Houlihan, J. M., P. A. Biro, H. M. Harper, H. J. Jenkinson, and C. H. Holmes. 1995. The human amnion is a site of MHC class lb expression: evidence for the expression of HLA-E and HLA-G. J Immunol. 154:5665-5674.

134. Hoyer, L. W., W. N. Drohan, and H. K. Lubon. 1994. Production of human therapeutic proteins in transgenic animals. Vox Sanguinis. 67:217-220.

135. Hudson, R. A., and C. D. Black. 1993. Novel Delivery Methods for Protein Drugs. American Pharmacy. NS33:23-24.

136. Hughes, M., A. Bassilakos, D. W. Andrews, G. Hortelano, J. W. Belmont, and P. L. Chang. 1994. Delivery of a secretable adenosine deaminase through microcapsuies—a novel approach to somatic gene therapy. Human Gene Therapy. 5:1445-1455.

137. Hunt, J., and H. Orr. 1992. HLA and maternal-fetal recognition. FASEB J. 6:2344-2348.

138. Hurwitz, D. R., M. Kirchgesser, W. Merrill, T. Galanopoulos, C. A. McGrath, S. Emami, M. Hansen, V. Cherington, J. M. Appel, C. B. Bizinkauskas, H. H. Brackman, P. H. Levine, and J. S. Greenberger. 1997. Systemic delivery of human growth hormone or human factor IX in dogs by reintroduced genetically modified autologous bone marrow stromal cells. Human Gene Therapy. 8:137-156.

139. Itoh, N., and S. Naga. 1993. A novel protein domain required for apoptosis: Mutational analysis of human Fas antigen. Journal of Biological Chemistry. 1993:10932-10937.

140. Jacquemin, P., E. Alsat, C. Oury, A. Belayew, M. Muller, D. Evain-Brion, and J. A. Martial. 1996. The enhancers of the human placental lactogen B, A and L genes: progressive activation during in vitro trophoblast differentiation and importance of the DF-3 element in determining their respective activities. DNA Cell Biol. 15:845-854.

141. Jaenisch, R. 1988. Transgenic animals. Science. 240:1468-1474.

142. Jaffe, G. J., K. Earnest, S. Fulcher, G. M. Lui, and L. L. Houston. 1990. Antitransferrin receptor immunotoxin inhibits proliferating human retinal pigment epithelial cells. Arch. Opthal. 108:1163-1168.

143. Janne, J., J. -M. Hyttinen, T. Peura, M. Tolvanen, L. Alhonen, and M. Halmekyto. 1992. Transgenic Animals as Bioproducers of Therapeutic Proteins. Annals of Medicine. 24:273-280.

144. Jansson, J. -O., K. Albertsson-Wikland, S. Eden, K. -G. Thomgren, and O. G. P. Isaksson. 1982. Effect of frequency of growth hormone administration on longitudinal bone growth and body weight in hypophysectomized rats. Acta Physiol Scand. 114:261-265.

145. Johnson, W., C. Albanese, S. Handwerger, T. Williams, R. G. Pestell, and J. L. Jameson. 1997. Regulation of the human chorionic gonadotropin alpha- and beta-subunit promoters by AP-2. J Biol Chem. 272:15405-15412.

146. Jomary, C., T. A. Piper, G. Dickson, L. A. Couture, A. E. Smith, M. Neal, and S. E. Jones. 1994. Adenovirus-mediated gene transfer to murine retinal cells in vitro and in vivo. FEBS Lett. 347:117-122.

147. Jones, K. R., and L. Reichardt. 1990. Molecular cloning of a human gene that is a member of the nerve growth factor family. Proc Natl Acad Sci USA. 87:8060-8064.

148. Jones, L. L., M. Oudega, M. B. Bunge, and M. H. Tuszynski. 2001. Neurotrophic factors, cellular bridges and gene therapy for spinal cord injury. J Physiol. 533:83-89.

149. Jun, F., T. Collier, and S. Y. Felten. 1994. Rat with partial unilateral nigrostriatal lesions as a model to study CNS plasticity. NeuroProtocol. 4:168-176.

150. Kalcheim, C., C. Carmeli, and A. Rosenthal. 1992. Neurotrophin 3 is a mitogen for cultured neural crest cells. Proc Natl Acad Sci USA. 89:1661-1665.

151. Kang, S. -M., D. B. Schneider, Z. Lin, D. Hanahan, D. A. Dichek, P. G. Stock, and S. Baekkeskov. 1997. Fas ligand expression in islets of Langerhans does not confer immune privilege and instead targets them for rapid destruction. Nature Medicine. 3:738-743.

152. Kaplitt, M. G., P. Leone, R. J. Samulski, X. Xiao, D. W. Pfaff, K. L. O'Malley, and M. J. During. 1994. Long-term gene expression and phenotypic correction using adeno-associated virus vectors in the mammalian brain. Nature Genet 8:148-154.

153. Kayagaki, N., A. Kawasaki, T. Ebata, H. Ohmoto, S. Ikeda, S. Inoue, K. Yoshino, K. Okumura, and H. Yagita. 1995. Metalloproteinase-mediated release of human Fas ligand. J Exp Med. 182:1777-1783.

154. Kelly, C. W., A. Janecki, A. Steinberger, and L. D. Russell. 1991. Structural characteristics of immature rat Sertoli cells in vivo and in vitro. Am J Anat 192:183-193.

155. Kessler, D. A., J. P. Siegel, P. D. Noguchi, K. D. Zoon, K. L. Feiden, and J. Woodcock. 1993. Regulation of somatic-cell therapy and gene therapy by the Food and Drug Administration. New Engl. J. Med. 329:1169-1173.

156. Kim, H. R., H. M. Lin, H. Biliran, and A. Raz. 1999. Cell cycle arrest and inhibition of anoikis by galectin-3 in human breast epithelial cells. Cancer Res. 59:4148-4154.

157. Kobayashi, N. R., D. P. Fan, K. M. Giehl, A. M. Bedard, S. J. Wiegand, and W. Tetzlaff. 1997. BDNF and NT-4/5 prevent atrophy of rat rubrospinal neurons after cervical axotomy, stimulate GAP-43 and Talphal-tubulin mRNA expression, and promote axonal regeneration. J Neurosci. 17:9583-9595.

158. Korbutt, G. S., J. F. Elliott, and R. V. Rajotte. 1997. Cotransplantation of allogeneic islets with allogeneic testicular cell aggreagtes allows long-term graft survival without systemic immunosuppression. Diabetes. 46:317-322.

159. Korbutt, G. S., W. L. Suarez-Pinzon, R. F. Power, R. V. Rajotte, and A. Rabinovitch. 2000. Testicular Sertoli cells exert both protective and destructive effects on syngeneic islet grafts in non-obese diabetic mice. Diabetologia. 43:474-480.

160. Kovats, S., E. Main, C. Librach, M. Stubblebine, S. Fisher, and R. DeMars. 1990. A class I antigen, HLA-G, expressed in human trophoblasts. Science. 248:220.

161. Kuehn, M. R., A. Bradley, E. J. Robertson, and M. J. Evans. 1987. A potential animal model for Lesch-Nyhan syndrome through introduction of HPRT mutations into mice. Nature. 326:295-298.

162. Kunkel-Bagden, E., H. N. Dai, and B. S. Bregman. 1993. Methods to assess the development and recovery of locomotor function after spinal cord injury in rats. Exp Neurol 119:153-164.

163. Kurachi, K., and E. W. Davie. 1982. Isolation and characterizatioin of a cDNA coding for human factor IX. Proc Natl Acad Sci USA. 79:6461-6464.

164. Kutty, R. K., C. N. Hagineni, G. Kutty, J. J. Hooks, G. J. Chader, and B. Wiggert. 1994. Increased expression of heme oxygenase-1 in human retinal pigment epithelial cells by transforming growth factor-beta. J Cell Physiol. 159:371-378.

165. Labosky, P. A., D. P. Barlow, and B. L. Hogan. 1994. Embryonic germ cell lines and their derivation from mouse primordial germ cells. Ciba Found Symp. 182:157-168.

166. Lau, H. T., M. Yu, A. Fontan, and C. J. Stoeckert Jr. 1996. Prevention of islet allograft rejection with engineered myoblasts expressing FasL in mice. Science. 273:109-112.

167. Ledley, F. D. 1989. Human gene therapy. In Biotechnology. G. Jacobson, editor. VCH Verlagsgesellschaft. 401-457.

168. Lee, V., A. Lee, E. Phillips, J. Roberts, and H. Weitlauf. 1998. Spatio-temporal pattern for expression of galectin-3 in the murine utero-placental complex: evidence for differential regulation. Biol Reprod. 58:1277-1282.

169. Lefebvre, S., P. Moreau, V. Guiard, E. C. Ibrahim, F. Adrian-Cabestre, C. Menier, J. Dausset, E. D. Carosella, and P. Paul. 1999. Molecular mechanisms controlling constitutive and IFN-gamma-inducible HLA-G expression in various cell types. J Reprod Immunol. 43:213-224.

170. Lehman, M., E. Graser, K. Risch, W. W. Hancock, A. Muller, B. Kuttler, H. -J. Hahn, J. W. Kupiec-Weglinski, J. Brock, and H. -D. Volk. 1997. Anti-CD4 monoclonal antibody-induced allograft tolerance in rats despicte persistence of donor-reactive T cells. Transplantation. 64:1181-1187.

171. Lenz, D. H., C. L. Weingart, and A. A. Weiss. 2000. Phagocytosed Bordetella pertussis fails to survive in human neutrophils. Infect Immun. 68:956-959.

172. Li, M. D., G. J. MacDonald, and J. J. Ford. 1997. Breed differences in expression of inhibin/activin subunits in porcine anterior pituitary glands. Endocrinology. 138:712-718.

173. Li, Z. Y., F. Wong, J. H. Chang, D. E. Possin, Y. Hao, R. M. Petters, and A. H. Milam. 1998. Rhodopsin transgenic pigs as a model for human retinitis pigmentosa. Invest Ophthal. 39:808-819.

174. Liu, N. -P., W. L. Roberts, L. P. Hale, M. C. Levesque, P. D. D, C. -L. Lu, and G. J. Jaffe. 1997. Expression of CD44 and variant isoforms in cultured human retinal pigment epithelial cells. Invest Ophthal Vis Sci. 38:2027-2037.

175. Lonberg, N., and R. M. Kay. 1997. Transgenic non-human animals capable of producing heterologous antibodies of various isotypes. U.S. Pat. No. 5,661,016.

176. Lu, S. C., W. -M. Sun, N. N. Chandrasekharam, J. J. Hooks, and R. Kannan. 1995. Bidirectional gluathione transport by cultured human retinal pigment epithelial cells. Invest Ophthal Vis Sci. 36:2523-2530.

177. Lucius, R., and R. Mentlein. 1995. Development of a culture system for pure rat neurons: advantages of a sandwich technique. Anat Anz. 177:447-454.

178. Lundberg, C., P. Horellou, J. Mallet, and A. Bjorklund. 1996. Generation of DOPA-Producing astrocytes by retroviral transduction of the human tyrosine hydroxylase gene: In vitro characterization and in vivo effects in the rat parkinson model. Exp Neurol. 139:39-53.

179. Lysiak, J. J., J. Hunt, G. A. Pringle, and P. K. Lala. 1995. Localization of transforming growth factor beta and its natural inhibitor decorin in the human placenta and decidua throughout gestation. Placenta. 16:221-231.

180. Majumdar, S. S., J. Tsuruta, M. D. Griswold, and A. Bartke. 1995. Isolation and culture of Sertoli cells from the testes of adult Siberian hamsters: analysis of proteins synthesized and secreted by Sertoli cells cultured from hamsters raised in a long or short photoperiod. Biol Reprod. 52:658-666.

181. Majumdar, S. S., S. J. Winters, and T. M. Plant. 1998. Procedures for the isolation and culture of Sertoli cells from the testes of infant, juvenile, and adult rhesus monkeys (Macaca mulatta). Biol Reprod. 58:633-640.

182. Malina, H., and X. Martin. 1993. Indoleamine 2,3-dioxygenase activity in the aqueous humor, iris/ciliary body, and retina of the bovine eye. Graefes Arch Clin Exp Ophthalmol. 231:482-486.

183. Malina, H., and X. Martin. 1996. Indoleamine 2,3-dioxygenase: antioxidant enzyme in the human eye. Graefes Arch Clin Exp Ophthalmol. 234:457-462.

184. Manninen, D. L., R. W. Evans, and M. K. Dugan. 1991. In Clinical Transplants. P. Terasaki, editor. UCLA Press, Los Angeles, Calif. 193.

185. Martin, D. M., D. Yee, and E. Feldman. 1992. Gene expression of the insulin-like growth factors and their receptors in cultured human retinal pigment epithelial cells. Brain Res. 12:181-186.

186. Martin, M. J., and C. A. Pinkert. 1994. Production of Transgenic Swine. In Transgenic Animal Technology: A Laboratory Handbook C. A. Pinkert, editor. Academic Press. 322-336.

187. Matarrese, P., O. Fusco, N. Tinari, C. Natoli, F. T. Liu, M. L. Semeraro, W. Malorni, and S. Iacobelli. 2000a. Galectin-3 overexpression protects from apoptosis by improving cell adhesion properties. Int J Cancer. 85:545-554.

188. Matarrese, P., N. Tinari, M. Semeraro, C. Natoli, S. Iacobelli, and W. Malorni. 2000b. Galectin-3 overexpression protects from cell damage and death by influencing mitochrondrial homeostatis. FEBS letters. 473:311-315.

189. Matsuo, T., I. Nasuda, T. Yasuda, and N. Matsuo. 1996. Gene Transfer to the Retina of Rat by Liposome Eye Drops. Biochem. Biophys. Res. Comm. 219:947-950.

190. McDonald, J. W. 1999. Repairing the damaged spinal cord. Sci Am. 281:64-73.

191. McMaster, M., C. Librach, Y. Zhou, K. Lim, M. Janatpour, R. DeMars, S. Kovats, C. Damsky, and S. Fisher. 1995. Human placental HLA-G expression is restricted to differentiated cytotophoblasts. J Immunol. 1545:3771-3778.

192. McMaster, M., Y. Zhou, S. Shorter, K. Kapasi, D. Geraghty, K. -H. Lim, and S. Fisher. 1998. HLA-G isoforms produced by placental cytotrophoblasts and found in amniotic fluid are due to unusual glycosylation. J Immunol. 160:5922-5928.

193. McTigue, D. M., P. J. Horner, B. T. Stokes, and F. H. Gage. 1998. Neurotrophin-3 and brain-derived neurotrophic factor induce oligodendrocyte proliferation and myelination of regenerating axons in the contused adult rat spinal cord. J Neurosci. 18:5354-5365.

194. Mesulam, M. -M. 1978. Tetramethyl benzidine for horseradish peroxidase neurohistochemistry: a non-carcinogenic blue reaction-product with superior sensitivity for visualizing neural afferents and efferents. J Histochem Cytochem. 26:106-117.

195. Microsoft, I. 1998. Eye Bank. In Encarta® 98 Encyclopedia. Microsoft, editor. Microsoft Corporation, Redmond, Wash.

196. Miller, A. D. 1996. Cell-surface receptors for retroviruses and implications for gene transfer. Proc. Natl. Acad. Sci. USA. 93:11407-11413.

197. Miller, A. D., and F. Chen. 1996. Retrovirus packaging cells based on 10A1 murine leukemia virus for production of vectors that use multiple receptors for cell entry. J. Virol. 70:5564-5571.

198. Miller, D. G., and A. D. Miller. 1994. A family of retroviruses that utilize related phosphate transporters for cell entry. J. Virol. 68:8270-8276.

199. Miller, L. K. 1997. Baculovirus interaction with host apoptotic pathways. Journal of Cell physiology. 173:178-182.

200. Miyoshi, H., M. Takahashi, F. H. Gage, and I. M. Verma. 1997. Stable and efficient gene transfer into the retina using an HIV-based lentiviral vector. Proc Natl Acad Sci USA. 94:10319-10323.

201. Mochii, M., Y. Mazaki, N. Mizuno, H. Hayashi, and G. Eguchi. 1998. Role of Mitf in differentiation and transdifferentiation of chicken pigmented epithelial cell. Dev Biol. 193:47-62.

202. Moffat, M., S. Harmon, J. Haycock, and K. L. O'Malley. 1994. L-Dopa and Dopamine-producing gene cassettes for gene therapy approaches to Parkinson's disease. Exper. Neurol. 144:69-73.

203. Möller, P., H. Walczak, S. Riedl, J. Strater, and P. H. Krammer. 1996. Panneth Cells Express High Levels of CD95 Ligand Transcripts. A Unique Property among Gastrointestinal Epithelia. Amer. J. Pathol. 149:9-13.

204. Monet-Kuntz, C., F. Guillou, I. Fontaine, and Y. Combarnous. 1992. Purification of ovine transferrin and study of the hormonal control of its secretion in enriched cultures of ovine Sertoli cells. J Repro Fertility. 94:189-201.

205. Montel, A. H., M. R. Bochan, W. S. Goebel, and Z. Brahmi. 1995. Fas-mediated cytotoxicity remains intact in perforin and granzyme B antisense transfectants of a human NK-like cell line. Cell Immunol. 165:312-317.

206. Moreau, P., P. Paul, N. Rouas-Freiss, M. Kirszenbaum, J. Dausset, and E. D. Carosella. 1998. Molecular and immunologic aspects of the nonclassical HLA class I antigen HLA-G: evidence for an important role in the maternal tolerance of the fetal allograft. Am J Reprod Immunol. 40:136-144.

207. Morgenstern, J. P., and H. Land. 1990. Advanced mammalian gene transfer: high titre retroviral cectors with multiple drug selection markers and a complementary helper-free packaging cell line. Nucleic Acids Res. 18:3587-3590.

208. Mountz, J. D., T. Zhou, and L. Johnson. 1990. Production of Transgenic Mice and Application to Immunology and Autoimmunity. Amer. J. Med. Sci. 299:322-329.

209. Munn, D., M. Zhou, J. Attwood, I. Bondarev, S. Conway, B. Marshall, C. Brown, and A. Mellor. 1998. Prevention of allogeneic fetal rejection by tryptophan

210. catabolism. Science. 281:1191-1193.

211. Murphy, D., and D. A. Carter. 1993. Transgenesis Techniques: Principles and Protocols. Humana Press Inc., Totowa, N. Mex.

212. Muzio, M., G. S. Salvesen, and V. M. Dixit. 1997. FLICE induced apoptosis in a cell-free system. Cleavae of caspase zymogens. Journal of Biological Chemistry. 272:2951-2956.

213. Nagineni, C., K. Pardhasaradhi, M. Martins, B. Detrick, and J. Hooks. 1996. Mechanisms of interferon-induced inhibition of Toxoplasma gondii replication in human retinal pigment epithelial cells. Infect Immun. 64:4188-4196.

214. Naldini, L., U. Bleomer, P. Gallay, D. Ory, R. Mulligan, F. H. Gage, I. M. Verma, and D. Trono. 1996. In vivo gene delivery and stable transduction of nondividing cells by a lentiviral vector. Science. 272:263-267.

215. Nehar, D., C. Mauduit, F. Boussouar, and M. Benahmed. 1997. Tumor necrosis factor-alpha-stimulated lactate production is linked to lactate dehydrogenase A expression and activity increase in porcine cultured Sertoli cells. Endocrinology. 138:1964-1971.

216. Newgard, C. B. 1998. Methods for producing insulin in response to noon-glucose secretagogues. U.S. Pat. No. 5,744,327.

217. Nicoletti, E., K. Kawase, and D. Thompson. 1998. Promoter analysis of RPE65, the gene encoding a 61-kDa retinal pigment epithelium-specific protein. Invest Opthal. Vis. Science. 39:637-644.

218. Noble, L. J., and J. R. Wrathall. 1985. Spinal cord contusion in the rat: morphometric analyses of alterations in the spinal cord. Exp Neurol. 88:135-149.

219. Noble, L. J., and J. R. Wrathall. 1989. Correlative analyses of lesion development and functional status after graded spinal cord contusive injuries in the rat. Exp Neurol. 103:34-40.

220. Noel, D., M. Pelegrin, M. Marin, M. Biard-Piechaczyk, J.C. Ourlin, J. C. Mani, and M. Piechaczyk. 1997. In vitro and in vivo secretion of cloned antibodies by genetically modified myogenic cells. Hum Gene Ther. 8:1219-1229.

221. O'Connell, J. 2000. Immune privilege or inflammation? The paradoxical effects of Fas ligand. Arch Immunol Ther Exp (Warsz). 48:73-79.

222. O'Malley, K. L., M. J. Anhalt, B. M. Martin, J. R. Kelsoe, S. L. Winfield, and E. I. Ginns. 1987. Isolation and characterization of the human tyrosine hydroxylase gene: identification of 5′ alternative splice sites responsible for multiple mRNAs. Biochemistiy. 26:2910-2914.

223. Oda, Y., and K. Kasai. 1983. Purification and characterization of beta-galactoside-binding lectin from chick embryonic skin. Biochim Biophys Acta. 761:237-245.

224. Olivero, D. K., and L. T. Furcht. 1993. Type IV collagen, laminin, and fibronectin promote the adhesion and migration of rabbit lens epithelial cells in vitro. Invest Ophthal Vis Sci. 34:2825-2834.

225. Orkin, S. H., and A. G. Motulsky. 1995. Report and Recommendations of the Panel to Assess the NIH Investment in Research on Gene Therapy.

226. Ortiz-Arduan, A., T. M. Danoff, R. Kalluri, S. Gonzalez-Cuadrado, S. L. Karp, K. Elkon, J. Egido, and E. G. Neilson. 1996. Regulation of Fas and Fas ligand expression in cultured murine renal cells and in the kidney during endotoxemia. Am J Physiol. 271:F1193-1201.

227. Ottonello, L., G. Tortolina, M. Amelotti, and F. Dallegri. 1999. Soluble Fas ligand is chemotactic for human neutrophilic polymorphonuclear leukocytes. J Immunol. 162:3601-3606.

228. Ozaki, Y., C. Nichol, and D. Duch. 1987. Utilization of dihydroflavin mononucleotide and superoxide anion for the decyclization of L-tryptophan by murine epididymal indoleamine 2,3- dioxygenase. Arch Biochem Biophys. 257:207-216.

229. Ozaki, Y., J. J. Reinhard, and C. Nichol. 1986. Cofactor activity of dihydroflavin mononucleotide and 230. tetrahydrobiopterin for murine epididymal indoleamine 231. 2,3-dioxygenase. Biochem Biophys Res Commun. 137:1106-1111.

232. Palmer, T. D., G. J. Rosman, W. R. Osborne, and A. D. Miller. 1991. Genetically modified skin fibroblasts persist long after transplantation but gradually inactivate introduced genes. Proc Natl Acad Sci USA. 88:1330-1334.

233. Palmiter, R. D., and R. L. Brinster. 1986. Germ-line transformation of mice. Ann Rev. Genet. 20:465-499.

234. Paxinos, G. 1995. The rat nervous system. Academic Press, San Diego, Calif.

235. Paxinos, G., and C. Watson. 1986. The rat brain in stereotaxic coordinates. Academic Press, San Diego.

236. Perandones, C. E., V. A. Illera, D. Peckham, L. L. Stunz, and R. F. Ashman. 1993. Regulation of apoptosis in vitro in mature murine spleen T cells. J Immunol. 151:3521-3529.

237. Phillips, B., K. Knisley, K. Weitlauf, J. Dorsett, V. Lee, and H. Weitlauf. 1996. Differential expression of two beta-galactoside-binding lectins in the reproductive tracts of pregnant mice. Biol Reprod. 55:548-558.

238. Piedrahita, J. A., K. Moore, C. Lee, B. Oetama, R. Weaks, J. Ramsoondar, J. Thomson, and J. Vasquez. 1997. Advances in the generation of transgenic pigs via embryo-dervied and primordial germ cell-dreived cells. J Reprod Fertil Suppl. 52:245-254.

239. Pinkert, C. A. 1994. Transgenic Animal Technology: A Laboratory Handbook. Academic Press.

240. Pognan, F., M. T. Masson, F. Lagelle, and C. Charuel. 1997. Establishment of a rat Sertoli cell line that displays the morphological and some of the functional characteristics of the native cell. Cell Biol Toxicology. 13:453-463.

241. Poirier, F., P. Timmons, M, C. T. Chan, J. L. Guenet, and P. W. Rigby. 1992. Expression of the L14 lectin during mouse embryogenesis suggests multiple roles during pre- and post-implantation development. Development. 115:143-155.

242. Potter, P. M., C. A. Pawlik, C. L. Morton, C. W. Naeve, and M. K. Danks. 1998. Isolation and partial characterization of a cDNA encoding a rabbit liver carrboxylesterase that activates the prodru irinotecan (CPT-11). Cancer Research. 58:2646-2651.

243. Ramesh, N., S. Lau, T. D. Palmer, R. Storb, and W. R. Osbome. 1993. High-level human adenosine deaminase expression in dog skin fibroblasts is not sustained following transplantation. Human Gene Therapy. 4.

244. Ramsdell, F., M. S. Seaman, R. E. Miller, K. S. Picha, M. K. Kennedy, and D. H. Lynch. 1994. Differential ability of Th1 and Th2 T cells to express Fas ligand and to undergo activation-induced cell death. InterNatl. Immunol. 6:1545-1553.

245. Renjifo, X., C. Howard, P. Kerkhofs, M. Denis, J. Urbain, M. Moser, and P. P. Pasoret. 1997. Purification and characterization of bovine dendritic cells from peripheral blood. Vet Immunol Immunopathol. 60:77-88.

246. Reventos, J., P. M. Sullivan, D. R. Joseph, and J. W. Gordon. 1993. Tissue-specific expression of the rat androgen-binding protein/sex hormone-binding globulin gene in transgenic mice. Mol Cell Endocrinol. 96:69-73.

247. Richmond, A., E. Balentien, H. G. Thomas, G. Flaggs, D. E. Barton, J. Speiss, R. Bordoni, U. Francke, and R. Derynck. 1988. Molecular characterization and chromosome mapping of melanoma growth stimulatory activity, a growth factor structurally related to beta-thromboglobulin. EMBO J. 7:2025-2033.

248. Rihova, B. 2000. Immunocompatibility and biocompatibility of cell delivery systems. Adv Drug Deliv Rev. 42:65-80.

249. Robert, P. Y., C. Lasalmonie, M. Cogne, J. P. Adenis, and M. Drouet. 1999. HLA-G and classical HLA class I transcripts in various components of the adult human eye. Eur J Immunogenet 26:271-274.

250. Robinson, I. C. A. F., and R. G. Clark. 1987. The Secretory pattern of GH and its significance for growth in the rat. In Growth Hormone: Basic and clinical aspects. O. Isaksson, Binder, C, Hall, K, Hokfelt, B, editor. Elservier, Amsterdam. 109-127.

251. Rogers, A., I. Boime, J. Connolly, J. Cook, and J. Russell. 1998. Maternal-fetal tolerance is maintained despite transgene-driven trophoblast expression of MHC class I, and defects in Fas and its ligand. Eur J Immunol. 28:3479-3487.

252. Rosenberg, S. A., P. Aebersold, K. Cornetta, A. Kasid, R. A. Morgan, R. Moen, K. E. M, M. T. Lotze, J. C. Yang, S. L. Topalian, M. J. Merino, K. Culver, A. D. Miller, R. M. Blaese, and W. F. Anderson. 1990. Gene transfer into humans: immunotherapy of patients with advanced melanoma, using tumor-infiltrating lymphocytes modified by retroviral gene transduction. N. Engl. J. Med. 323:570-578.

253. Roth, I., D. Corry, R. Locksley, J. Abrams, M. Litton, and S. Fisher. 1996. Human placental cytotrophoblasts produce the immunosuppressive cytokine interleukin 10. J Exp Med. 184:539-548.

254. Rouas-Freiss, N., I. Khalil-Daher, R. Marchal-Bras Goncalves, C. Menier, J. Dausset, and E. D. Carosella. 1999. Role of HLA-G in maternal-fetal immune tolerance. Transplant Proc. 31:724-725.

255. Rouas-Freiss, N., P. Paul, J. Dausset, and E. D. Carosella. 2000. HLA-G promotes immune tolerance [In Process Citation]. J Biol Regul Homeost Agents. 14:93-98.

256. Rouvier, E., M. F. Luciani, and P. Golstein. 1993. Fas involvement in Ca(2+)-independent T cell-mediated cytotoxicity. J. Exp. Med. 177:95-200.

257. Runic, R., C. Lockwood, L. LaChapelle, B. Dipasquale, R. Demopoulos, A. Kumar, and S. Guller. 1998. Apoptosis and Fas expression in human fetal membranes. J Clin Endocrinol Metab. 83:660-666.

258. Runic, R., C. J. Lockwood, Y. Ma, B. Dipasquale, and S. Guller. 1996. Expression of fas ligand by human cytotrophoblasts: implications in placentation and fetal survival. J Clin Endocrinol Metab. 81:3119-3122.

259. Sadick, M. D., A. Galloway, D. Shelton, V. Hale, S. Weck, V. Anicetti, and W. L. T. Wong. 1997. Analysis of neurotrophin/receptor interactions with a gD-flag-modified quantitative kinase receptor activation (gD.KIRA) enzume-linked immunosorbant assay. Exp Cell Res. 234:354-361.

260. Sakagami, K., H. Naka, A. Hayashi, M. Kamei, T. Sasabe, and Y. Tano. 1995. A rapid method for isolation of retinal pigment epithelial cells from rat eyeballs. Ophthalmic Res. 27:262-267.

261. Sanberg, P. R., C. V. Borlongan, A. I. Othberg, S. Saporta, T. B. Freeman, and D. F. Cameron. 1997. Testis-derived Sertoli cells have a trophic effect on dopamine neurons and alleviate hemiparkinsonism in rats. Nature Medicine. 3:1129-1132.

262. Sanders-Sanchez, S. R., D. Malsbury, and A. T. Tsin. 1990. Comparison of retinal pigment epithelium cell preparations from the bovine eye. Experientia. 46:498-500.

263. Saporta, S., D. F. Cameron, C. V. Borlongan, and P. R. Sanberg. 1997. Survival of rat and porcine Sertoli cell transplants in the rat striatum without Cyclosporine-A immunosuppression. Exp Neurol. 146:299-304.

264. Sasaki, H., X. C. Xu, and T. Mohanakumar. 1999a. HLA-E and HLA-G expression on porcine endothelial cells inhibit xenoreactive human NK cells through CD94/NKG2-dependent and -independent pathways. J Immunol. 163:6301-6305.

265. Sasaki, H., X. C. Xu, D. M. Smith, T. Howard, and T. Mohanakumar. 1999b. HLA-G expression protects porcine endothelial cells against natural killer cell-mediated xenogeneic cytotoxicity. Transplantation. 67:31-37.

266. Satoh, T., M. Hosokawa, Atsumi R, W. Suzuki, H. Hakusui, and E. Nagai. 1994. Methabolic activation of CPT-11, 7-ethyl-10-[4-(1-piperidine)-1-piperidine]carbonyloxycamptothecin, a novel antitumor agent, by carboxylesterase. Biological Pharmaceutics Bulletin. 17:662-664.

267. Schmidt, R. H., M. Ingvar, O. Lindvall, U. Stenevi, and A. Bjorklund. 1982. Functional activity of substatia nigra grafts reinnervating the striatum: Neurotransmitter metabolism and [14C]2-deoxy-d-glucose autoradiography. J Neurochem. 38:737-748.

268. Schnieke, A. E., A. J. Kind, W. A. Ritchie, K. Mycock, A. R. Scott, M. Ritchie, I. Wilmut, A. Colman, and K. H. Campbell. 1997. Human factor IX transgenic sheep produced by transfer of nuclei from transfected fetal fibroblasts. Science. 278:2130-2133.

269. Schubert, C. A., H. Kimura, C. Spee, D. R. Hinton, E. M. Gordon, W. F. Anderson, and S. J. Ryan. 1998. Retrovirus-mediated transfer of the suicide gene into retinal pigment epithelial cells in vitro. Current Eye Res. 16:656-662.

270. Selawry, H. P., and D. F. Cameron. 1993. Sertoli Cell-Enriched Fractions in Successful Islet Cell Transplantation. Cell Transplantation. 2:123-129.

271. Selawry, H. P., X. Wang, and L. Alloush. 1996. Sertoli cell-induced defects on functional and structural characteristics of isolated neonatal porcine islets. Cell Transplantation. 5:517-524.

272. Seldon, R. F., K. B. Howie, M. E. Rowe, H. M. Goodman, and D. D. Moore. 1986. Human growth hormone as a reporter gene in regulation studies employing transient gene expression. Mol Cell Biol. 6:3173-3179.

273. Senut, M. -C., M. H. Tuszynski, H. K. Raymon, S. T. Suhr, N. H. Liou, K. R. Jones, L. F. Reichardt, and F. H. Gage. 1995. Regional differences in responsiveness of adult CNS axons to grafts of cells expressing human neurotrophin 3. Exp. Neurol. 135:36-55.

274. Seshagiri, S., and L. K. Miller. 1997. Baculovirus inhibitors of apoptosis (IAPs) block activation of Sf-caspase-1. Proceedings of the National Academy of Sciences, United States of America. 94:13606-13611.

275. Sharma, R. K. 1998. Mouse trophoblastic cell lines: I—Relationship between invasive potential and TGF-beta 1. In Vivo. 12:431-440.

276. Shi, S. R., M. E. Key, and K. L. Kalra. 1991. Antigen retrieval in formalin-fixed, paraffin-embedded tissues: an enhancement method for immunohistochemical staining based on microwave oven heating of tissue sections. J Histochem Cytochem. 39:741-748.

277. Siegling, A., M. Lehmann, C. Platzer, F. Emmrich, and H. D. Volk. 1994. A novel and multispecific competitor for quantitative PCR analysis of cytokine gene expression in rats. J Immunol Meth. 177:23.

278. Skottner, A., R. G. Clark, L. Fryklund, and I. C. A. F. Robinson. 1989. Growth responses in a mutant dwarf rat to human growth hormone and recombinant human insulin-like growth factor 1. Endocrinology. 124:2519-2525.

279. Smith, G. M., J. Hale, E. M. Pasnikowski, R. M. Linksay, V. Wong, and J. S. Rudge. 1996. Astrocytes infected with replication=defective adenovirus containing a secreted form of CNTF or NT3 show enhanced support of neuronal populations in vitro. Exp Neurol. 139:156-166.

280. Southern, P. J., and P. Berg. 1982. Transformation of mammalian cells to antibiotic resistance with a bacterial gene under control of the SV40 early region promoter. J. Mol. Appl. Genet. 1:327-341.

281. Squinto, S. P., J. A. Madri, S. Kennedy, and J. Springhorn. 1994. The ENCEL system, a somatic cell protein delivery system. In Vivo. 8:771-780.

282. St Louis, D., and I. M. Verma. 1988. An alternative approach to somatic cell gene therapy. Proc NAtl Acad Sci USA. 85:3150-3154.

283. Steeves, J. D., and W. Tetzlaff. 1998. Engines, accelerators, and brakes on functional spinal cord repair. Ann N Y Acad Sci. 860:412-424.

284. Stichel, C. C., and H. W. Muller. 1998. Experimental strategies to promote axonal regeneration after traumatic central nervous system injury. Prog Neurobiol. 56:119-148.

285. Strasser, A., A. W. Harris, D. C. S. Huang, P. H. Krammer, and S. Cory. 1995. Bcl-2 and Fas/APO-1 regulate distinct pathways to lymphocyte apoptosis. The EMBO Journal. 14:6136-6147.

286. Streilein, J., D. Bradley, Y. Sano, and Y. Sonoda. 1996. Immunosuppressive properties of tissues obtained from eyes with experimentally manipulated comeas. Invest Ophthalmol Vis Sci. 37:413-424.

287. Streilein, J. W. 1995. Unraveling Immune Privilege. Science. 270:1158-1159.

288. Suarez-Pinzon, W., G. S. Korbutt, R. Power, J. Hooton, R. V. Rajotte, and A. Rabinovitch. 2000. Testicular sertoli cells protect islet beta-cells from autoimmune destruction in NOD mice by a transforming growth factor-betal -dependent mechanism [In Process Citation]. Diabetes. 49:1810-1818.

289. Suda, T., and S. Nagata. 1994. Purification and characterization of the Fas-ligand that induces apoptosis. J Exp Med. 179:873-879.

290. Suda, T., T. Okazaki, Y. Naito, T. Yokota, N. Arai, S. Ozaki, K. Nakao, and S. Nagata. 1995. Expression of the Fas ligand in cells of T cell lineage. J. Immunol. 154:3806-3813.

291. Suda, T., T. Takahashi, P. Golstein, and S. Nagata. 1993. Molecular cloning and expression of the Fas ligand: a novel member of the tumor necrosis factor family. Cell. 75:1169-1178.

292. Sutkowski, N., M. -L. Kuo, A. Varela-Echavarria, J. P. Dougherty, and Y. Ron. 1994. A murine model for B-lymphocyte somatic cell gene therapy. Proceedings of the National Academy of Sciences, United States of America. 91:8875-8879.

293. Tai, I. T., and A. M. Sun. 1993. Microencapsulation of recombinant cells: a new delivery system for gene therapy. Faseb J. 7:1061-1069.

294. Takahashi, T., M. Tanaka, J. Inazawa, T. Abe, S. Takashi, and S. Nagata. 1994. Human Fas ligand: gene structure, chromosomal location and species specificity. Int Immunol. 6:1567-1574.

295. Takahasi, T., M. Tanaka, C. I. Brannan, N. A. Jenkins, N. G. Copeland, T. Suda, and S. Nagata. 1994. Generalized Lymphoproliferative Disease in Mice, Caused by a Point Mutation in the Fas Ligand. Cell. 76:969-976.

296. Takeuchi, T., H. Suzuki, S. Sakurai, H. Nogami, S. Okuma, and H. Ishikawa. 1990. Molecular mechanism of growth hormone (GH) deficiency in the spontaneous dwarf rat: detection of abnormal splicing of GH nessenger ribonucleic acid by the polymerase chain reaction. Endocrinology. 126:31-38.

297. Takikawa, O., T. Kuroiwa, F. Yamazaki, and R. Kido. 1988. Mechanism of interferon-gamma action. Characterization of indoleamine 2,3-dioxygenase in cultured human cells induced by interferon-gamma and evaluation of the enzyme-mediated tryptophan degradation in its anticellular activity. J Biol Chem. 263:2041-2048.

298. Tanaka, S., T. Kunath, A. -K. Hadjantonakis, A. Nagy, and J. Rossant. 1998. Promotion of trophoblast stem cell proliferation by FGF4. Science. 282:2072-2075.

299. Tanihara, H., M. Yoshida, M. Matsumoto, and N. Yoshimura. 1993. Identification of transforming growth factor-beta expressed in cultured human retinal pigment epithelial cells. Invest Ophthalmol Vis Sci. 34:413-419.

300. Taylor, M., and G. Feng. 1991. Relationship between interferon-gamma, indoleamine 2,3-dioxygenase, and tryptophan catabolism [see comments]. FASEB J. 5:2516-2522.

301. Tewari, M., and V. M. Dixit. 1995. Fas- and tumor necrosis factor-induced apoptosis is inhibited by the poxvirus crmA gene product. Journal of Biological Chemistry. 270:3255-3260.

302. Tezel, T. H., L. V. Del Priore, and H. J. Kaplan. 1997. Harvest and storage of adult human retinal pigment epithelial sheets. Current Eye Research. 16:802-809.

303. Thomgren, K. G., and L. I. Hansson. 1974. Determination of longitudinal bone growth with tetracycline in hypophysectomized rats. Acta Endocrinol (Copenh) 75:653.

304. Turvey, S. E., V. Gonzalez-Nicolini, C. I. Kingsley, A. T. Larregina, P. J. Morris, M. G. Castro, P. R. Lowenstein, and K. J. Wood. 2000. Fas ligand-transfected myoblasts and islet cell transplantation. Transplantation. 69:1972-1976.

305. Tuszynski, M. H., K. Gabriel, F. H. Gage, S. Suhr, S. Meyer, and A. Rosetti. 1996. Nerve growth factor delivery by gene transfer induces differential outgrowth of sensory, motor and noradrenergic neurites after adult spinal cord injury. Exp Neurol. 137:157-173.

306. Uludag, H., and M. V. Sefton. 1992. Metabolic activity of CHO fibroblasts in HEMA-MMA microcapsules. Biotechnol. Bioeng. 39:672-678.

307. van der Burg, M. P., I. Basir, and E. Bouwman. 1998. No porcine islet loss during density gradient purification in a novel iodixanolin University of Wisconsin solution. Transpl Proc. 30:362-363.

308. Vogt, P. M., S. Thompson, C. Andree, P. Liu, K. Breuing, D. Hatzis, H. Brown, R. C. Mulligan, and E. Eriksson. 1994. Genetically modified keratinocytes transplanted to wounds reconstitute the epidermis. Proc Natl Acad Sci USA. 91:9307-9311.

309. Wang, H., P. A. Tresco, P. Aebischer, and J. Sagen. 1991. Pain reduction by transplants of polymer encapsulated bovine chromaffin cells in the rat spinal subarachnoid space. Soc. Neurosci. Abstr. 17:234.

310. Wang, X., S. Zinkel, K. Polonsky, and E. Fuchs. 1997. Transgenic studies with a keratin promoter-driven growth hormone transgene: Prospects for gene therapy. Proc. Natl. Acad. Sci. USA. 94:219-226.

311. Wang, Y., B. Detrick, and J. J. Hooks. 1993. Coronavirus (JMH) replication within the retina: analysis of cell tropism in mouse retinal cell cultures. Virology. 193:124-137.

312. Wearley, L. L. 1991. Recent Progress in Protein and Peptide Delivery by Noninvasive Routes. Critical Reviews in Therapeutic Drug Carrier Systems. 8:331-394.

313. Wenkel, H., and J. W. Streilein. 2000. Evidence that retinal pigment epithelium functions as an immune-privileged tissue. Invest Ophthalmol Vis Sci. 41:3467-3473.

314. Wigler, M., S. Silverstein, L. S. Lee, A. Pellicer, Y. C. Cheng, and R. Axel. 1977. Transfer of purified herpes virus thymidine kinase gene to cultured mouse cells. Cell. 11:223-232.

315. Williams, S. R., F. C. Ousley, L. J. Vitez, and R. B. DuBridge. 1994. Rapid detection of homologous recombinants in nontransformed human cells. Proc. Nati. Acad. Sci. USA. 91:11943-11947.

316. Wilson, S. E., Q. Li, J. Weng, P. A. Barry-Lane, J. V. Jester, Q. Liang, and R. J. Wordinger. 1996. The Fas-Fas ligand system and other modulators of apoptosis in the cornea. Invest. Opthal. and Vis. Sci. 37:1582-1592.

317. Wistow, G. J., M. P. Shaughnessey, D. C. Lee, J. Hodin, and P. S. Zelenka. 1993. A macrophage migration inhibitory factor is expressed in the deifferentiating cells of the eye lens. Proc Natl Acad Sci USA. 90:1272-1275.

318. Wolff, J. A. 1994. Gene Therapeutics: Methods and Applications of Direct Gene Transfer. Birkhauser, Boston, Mass.

319. Wollina, U., G. Schreiber, M. Gornig, S. Feldrappe, M. Burchert, and H. J. Gabius. 1999. Sertoli cell expression of galectin-1 and -3 and accessible binding sites in normal human testis and Sertoli cell only-syndrome. Histol Histopathol. 14:779-784.

320. Wrathall, J. R., R. K. Pettegrew, and F. Harvey. 1985. Spinal cord contusion in the rat: production of graded, reproducible, injury groups. Exp Neurol. 88:108-122.

321. Xerri, L., E. Devilard, J. Hassouri, C. Mawas, and F. Birg. 1997. Fas ligand is not only expressed in immune privileged human organs but is also coexpressed with Fas in various epithelial tissues. Journal of Clinical Pathology: Molecular Pathology. 50:87-91.

322. Yamazaki, F., T. Kuroiwa, O. takikawa, and R. Kido. 1985. Human indolylamine 2,3-dioxygenase: its tissue distribution, and characterization of the placental enzyme. Biochem J. 230:635-638.

323. Yang, R. -Y., D. Hsu, and F. -T. Liu. 1996. Expression of galectin-3 modulates T-cell growth and apoptosis. Proc Natl Acad Sci USA. 93:6737-6742.

324. Yoshimoto, Y., Q. Lin, T. J. Collier, D. M. Frim, X. O. Breakefield, and M. C. Bohn. 1995. Astrocytes retrovi rally transduced with BDNF elicit behavioral improvement in a rat model of Parkinson's disease. Brain Res. 691:25-36.

325. Zaidi, A., M. Schmoeckel, F. Bhatti, P. Waterworth, M. Tolan, E. Cozzi, G. Chavez, G. Langford, S. Thiru, J. Wallwork, D. White, and P. Friend. 1998. Life-supporting pig-to-primate renal xenotransplantation using genetically modified donors. Transplantation. 65:1584-1590.

What is claimed is:

1. A method of providing a biologically active moiety by administering cells that are naturally immune privileged and that have been isolated and genetically modified in a laboratory apparatus so as to express said biologically active moiety such that said cells express said biologically active moiety in pharmacologically effective amounts in vivo.

2. The method of claim 1 wherein said immune-privileged cells are derived from one of the tissues of the eye consisting of the iris, ciliary body, retina, and comeal endothelium.

3. The method of claim 1 where said immune privileged cells are Sertoli cells of the testes.

4. The method of claim 1 wherein said immune-privileged cells are from one of a group of cell types of the placenta consisting of trophoblasts, decidual cells, endometrial glandular epithelial cells, and endothelial cells.

5. The method of claim 1 where said immune-privileged cells are from one of a group of cells of the immune system consisting of T lymphocytes, B lymphocytes, natural killer cells, and macrophages.

6. The method of claim 1, where said immune-privileged cells are Paneth cells of gastrointestinal epithelium.

7. The method of claim 1, wherein said genetic modification is a nonviral physical method selected from the group including but not limited to microinjection, electroporation, lipofection, and chemically-mediated transfection with calcium phosphate or liposomes.

8. The method of claim 1, wherein said genetic modification uses one or more viral vectors selected from the group including but not limited to retroviral vectors, adenoviral vectors, and, adeno-associated viral vectors.

9. The method of claim 1, wherein said administration is selected from the group of methods consisting of intravenous, intramuscular, intraperitoneal, and subcutaneous injection and infusions.

10. The method of claim 1, wherein said cells are administered by surgical implantation.

11. The method of claim 1, wherein said cells are administered to the central nervous system.

12. A composition comprising cells that are naturally immune privileged and that been isolated and genetically modified in a laboratory apparatus to express said biologically active moiety such that said cells express said biologically active moiety in pharmacologically effective amounts in vivo.

13. The composition of claim 12, wherein said biologically active moiety is not naturally expressed by said cells.

14. The composition of claim 12, wherein said biologically active moiety is naturally expressed by said cells in less than pharmacologically effective amounts.

15. The composition of claim 12, wherein said immune-privileged cells or tissues are non-human cells or tissues.

16. The composition of claim 12, wherein said immune-privileged cells or tissues are human cells or tissues.

17. The composition of claim 12, wherein said immune-privileged cells or tissues are primary cells.

18. The composition of claim 12, wherein said immune-privileged cells or tissues are immortalized cells.

19. The composition of claim 12, wherein said immune-privileged cells are progenitor stem cells.

20. The composition of claim 12, wherein said immune-privileged cells or tissues have been passaged one or more times.

21. The composition of claim 12, wherein said immune-privileged cells are obtained from a transgenic non-human animal or the descendent of the said transgenic non-human animal who has had DNA introduced at an embryonic state such that said immune-privileged cells express a biologically active moiety in pharmacologically effective amounts.

22. The method of claim 12, wherein said immune-privileged cells are adherent to a biologically inert material.

23. The composition of claim 12, wherein said biologically active moiety is selected from the group including but not limited to insulin, Clotting Factors II, VII, VIII, IX, X, vasopressin, adenosine deaminase, glucocerebrosidase, human growth hormone, erythropoietin, calcitonin, leptin, interferon alpha, interferon beta, granulocyte colony-stimulating factor, granulocyte-macrophage colony stimulating factor, gangliosides, interleukins, cytokines, and antibodies.

24. The composition of claim 12, wherein said biologically active moiety is selected from a group of molecules therapeutic for neurological diseases and conditions, including but not limited to neurotrophins, neurotrophic factors, proteins that stimulate axonal growth, and neurotransmitters.


SEQUENCE LISTING (9 sequences)

SEQ ID NO: 1 - DNA, 19 bp, artificial sequence
  primer
tacgcggagcataagagtc

SEQ ID NO: 2 - DNA, 18 bp, artificial sequence
  primer
ctcccccagcaccgtgac

SEQ ID NO: 3 - DNA, 21 bp, artificial sequence
  primer
gctcttccagccctccttcct

SEQ ID NO: 4 - DNA, 24 bp, artificial sequence
  primer
ttgtaaccaactgggacgatatgg

SEQ ID NO: 5 - DNA, 24 bp, artificial sequence
  primer
gatcttgatcttcatggtgctagg

SEQ ID NO: 6 - DNA, 20 bp, artificial sequence
  primer
agaacattgatgatggcacc

SEQ ID NO: 7 - DNA, 20 bp, artificial sequence
  primer
atatccacagagtaccttgt

SEQ ID NO: 8 - DNA, 20 bp, artificial sequence
  primer
tagctgcaatggtacaggct

SEQ ID NO: 9 - DNA, 25 bp, artificial sequence
  primer
ttaggaggtcatagacgttgctgtc